(19) United States
(12) Patent Application Publication
(10) Pub. No.: US 2024/0352535 A1
(43) Pub. Date: Oct. 24, 2024

(54) Methods and Systems for Prostate Cancer Characterization and Treatment

(71) Applicants: THE REGENTS OF THE UNIVERSITY OF CALIFORNIA, Oakland, CA (US); University Health Network, Toronto (CA)

(72) Inventors: Paul C. BOUTROS, Los Angeles, CA (US); Michael FRASER, Toronto (CA)

(73) Assignees: THE REGENTS OF THE UNIVERSITY OF CALIFORNIA, Oakland, CA (US); University Health Network, Toronto, ON (CA)

(21) Appl. No.: 18/684,427

(22) PCT Filed: Aug. 17, 2022
(86) PCT No.: PCT/US2022/075089

§ 371 Date: Feb. 16, 2024

Related U.S. Application Data
(60) Provisional application No. 63/234,126, filed on Aug. 17, 2021.

(51) Int. Cl. C12Q 1/6886

(52) U.S. Cl. CPC C12Q 1/6886; C12Q 2600/106; C12Q 2600/118; C12Q 2600/154; C12Q 2600/156; C12Q 2600/158

(57) ABSTRACT

Disclosed herein are methods and compositions for treatment, prognosis, and diagnosis of cancer, including prostate cancer. Aspects of the disclosure are directed to methods for a subject having prostate cancer determined to have ZNRF3 genomic loss, reduced ZNRF3 expression, and/or increased ZNRF3 methylation. Also disclosed are methods for analysis of tumor DNA for ZNRF3 copy number status, expression, and/or methylation, as well as compositions and kits useful for such analysis.

### CROSS-REFERENCE TO RELATED APPLICATIONS

This application claims benefit of priority to U.S. Provisional Application No. 63/234,126, filed Aug. 17, 2021, which is hereby incorporated by reference in its entirety.

### STATEMENT REGARDING FEDERAL SUPPORT

This invention was made with government support under Grant Numbers CA016042, CA214194 and CA248265, awarded by National Institutes of Health. The government has certain rights in the invention.

### BACKGROUND

### I. Field of the Invention

Aspects of this invention relate to the fields of cancer biology, genetics, and medicine.

### II. Background

While the vast majority of prostate cancers are organ-confined at diagnosis¹, a significant proportion of these tumours relapse following surgery or radiation therapy^{2,3}. This necessitates salvage therapy to prevent or limit development of distant metastases⁴. For example, up to 20% of men with intermediate-grade prostate cancer will experience biochemical relapse within three years of definitive local therapy^{5,6}, which portends an aggressive clinical course. There is therefore a clear need to improve on current risk-stratification guidelines, which rely on three clinical prognostic factors (i.e. Gleason/ISUP grade, pre-treatment serum concentration of prostate-specific antigen (PSA), and clinical T category).

Many somatic mutations have been proposed to predict relapse of localized prostate cancer⁷⁻¹⁴. However, the majority of these studies focus on weak surrogates of disease-specific mortality (e.g., biochemical relapse; BCR). Moreover, while genome-wide analysis offers an unbiased approach to biomarker discovery, discovering, characterizing, and validating these biomarkers is limited by the high false discovery rate that results from simultaneous testing of multiple mutations for association with clinical outcome. Mutations associated with lethal disease are evolutionarily selected for in localized tumors¹⁵. This suggests that mutations that are highly prevalent in lethal metastatic, castration-resistant prostate cancer (mCRPC) but rare in localized disease may be prognostic biomarkers that reflect elevated risk of occult metastatic disease. While there are some data comparing the prevalence of driver mutation prevalence in localized disease vs. mCRPC^{16,17}, a comprehensive analyses of the clinical impact of this differential is lacking.

There exists a need for new and improved methods for diagnosis, prognosis, and informed treatment of cancer, including prostate cancer, based on analysis of tumor mutations, expression, and epigenetics.

### SUMMARY

Aspects of the present disclosure provide methods, systems, and compositions useful in diagnosis, prognosis, and treatment of cancer, including prostate cancer, based on genomic analysis. Accordingly, disclosed herein are methods for diagnosis of a subject as having high risk or very high risk prostate cancer based on identification of genomic loss, reduced expression, and/or increased methylation of ZNRF3. Also disclosed are methods for treatment of a subject with prostate cancer comprising admininstering an effective amount of a prostate cancer therapy, such as an aggressive prostate cancer therapy (e.g., radiotherapy and hormone therapy) to a subject determined to have ZRNF3 genomic loss, reduced expression of ZNRF3, and/or increased methylation of ZNRF3 in a prostate cancer sample from the subject. Further disclosed are compositions and kits useful in analysis and characterization of ZNRF3 from a prostate cancer sample.

Aspects of the present disclosure include methods for cancer diagnosis, methods for cancer treatment, methods for cancer prognosis, methods for preventing cancer, methods for predicting cancer occurrence, methods for predicting cancer characteristics, methods for characterizing cancer, methods for identifying a subject as having cancer, methods for diagnosing a subject with prostate cancer, methods for determining a prostate cancer patient has aggressive prostate cancer, methods for determining a treatment plan for prostate cancer, methods for genomic analysis of a prostate cancer sample, methods for analysis of prostate cancer DNA, methods for detecting a genetic mutation (e.g., structural variation, copy number alteration, single nucleotide variation, etc.) from a prostate cancer sample, methods for detecting copy number variation, methods for detecting genomic loss, methods for detecting genomic gain, methods for assaying ZNFR3 transcript or protein levels, methods for assaying methylation of one or more loci on ZNFR3, and methods for evaluating a risk of developing cancer. Methods of the present disclosure can include at least 1, 2, 3, 4, or more of the following steps: obtaining a biological sample from a subject, isolating nucleic acids from a subject, sequencing nucleic acids from a subject, amplifying nucleic acids from a subject, isolating tumor DNA from a subject, sequencing tumor DNA from a subject, isolating tumor RNA from a subject, sequencing tumor RNA from a subject, obtaining a prostate cancer sample from a subject, detecting a mutation in a gene from a prostate cancer sample, detecting a mutation of Table 4 from a prostate cancer sample, detecting a copy number variation in a gene from a prostate cancer sample, detecting genomic loss of a gene (e.g., ZNRF3) from a prostate cancer sample, detecting genomic gain of a gene (e.g., CCND1) from a prostate cancer sample, detecting reduced expression of a gene (e.g., ZNRF3) from a prostate cancer sample, detecting increased expression of a gene from a prostate cancer sample, detecting increased methylation of a gene (e.g., ZNRF3) from a prostate cancer sample, detecting decreased methylation of a gene from a prostate cancer sample, and administering a cancer therapy to a subject. Any one or more of the preceeding steps may be excluded from certain aspects of the disclosure.

Aspects of the disclosure are directed to methods for treating a subject for prostate cancer determined to have a mutation (e.g., copy number variation), altered expression, and/or altered methylation of one or more prognostic genes. Certain prognostic genes are disclosed herein including, for example, those listed in Tables 1-12. In some aspects, a prognostic gene of the disclosure is a gene listed in Table 4. In some aspects, a subject is determined to have one or more mutations listed in Table 4 from a prostate cancer sample. In some aspects, a prognostic gene of the disclosure is ZNRF3. Accordingly, disclosed herein, in some aspects, is a method for treating a subject for prostate cancer comprising administering an effective amount of a prostate cancer therapy (e.g., aggressive therapy such as radiotherapy and hormone therapy) to a subject determined to have (a) genomic loss, (b) reduced expression, and/or (c) increased methylation of ZNRF3 in a prostate cancer sample from the subject. Also disclosed are methods for diagnosing a subject as having high risk or very high risk prostate cancer comprising detecting (a) genomic loss, (b) reduced expression, and/or (c) increased methylation of ZNRF3 in a prostate cancer sample from the subject.

Disclosed herein, in some aspects, is a method for treating a subject for prostate cancer, the method comprising administering an effective amount of a prostate cancer therapy to a subject determined to have ZNRF3 genomic loss in a prostate cancer sample from the subject. Also disclosed, in some aspects, is a method for treating a subject for prostate cancer, the method comprising: (a) detecting ZNRF3 genomic loss in a prostate cancer sample from the subject; and (b) administering an effective amount of a prostate cancer therapy to the subject. In some aspects, the subject was determined to have ZNRF3 genomic loss by sequencing DNA from the prostate cancer sample. In some aspects, the subject was determined to have ZNRF3 genomic loss by polymerase chain reaction analysis of DNA from the prostate cancer sample. In some aspects, the subject was determined to have ZNRF3 genomic loss by microarray analysis of DNA from the prostate cancer sample. In some aspects, the subject was determined to have ZNRF3 genomic loss by in situ hybridization analysis of DNA from the prostate cancer sample.

Disclosed herein, in some aspects, is a method for treating a subject for prostate cancer, the method comprising administering an effective amount of a prostate cancer therapy to a subject determined to have reduced ZNRF3 expression in a prostate cancer sample from the subject. Also disclosed, in some aspects, is a method for treating a subject for prostate cancer, the method comprising: (a) detecting reduced ZNRF3 expression in a prostate cancer sample from the subject; and (b) administering an effective amount of a prostate cancer therapy to the subject. In some aspects, the reduced ZNRF3 expression is reduced ZNRF3 RNA. In some aspects, the subject was determined to have reduced ZNRF3 expression by sequencing RNA from the prostate cancer sample. In some aspects, the reduced ZNRF3 expression is reduced ZNRF3 protein. In some aspects, the subject was determined to have reduced ZNRF3 expression by measuring ZNRF3 protein levels from the prostate cancer sample.

Further disclosed herein, in some aspects, is a method for treating a subject for prostate cancer, the method comprising administering an effective amount of a prostate cancer therapy to a subject determined to have increased ZNRF3 methylation in a prostate cancer sample from the subject. Also disclosed, in some aspects, is a method for treating a subject for prostate cancer, the method comprising: (a) detecting increased ZNRF3 methylation in a prostate cancer sample from the subject; and (b) administering an effective amount of a prostate cancer therapy to the subject. In some aspects, the subject was determined to have increased ZNRF3 methylation by sequencing DNA from the prostate cancer sample. In some aspects, the sequencing comprised bisulfite sequencing. In some aspects, the subject was determined to have increased ZNRF3 methylation by polymerase chain reaction analysis of DNA from the prostate cancer sample. In some aspects, the subject was determined to have increased ZNRF3 methylation by microarray analysis of DNA from the prostate cancer sample. In some aspects, the subject was determined to have increased ZNRF3 methylation by in situ hybridization analysis of DNA from the prostate cancer sample.

Disclosed herein, in some aspects, is a method for method for prostate cancer prognosis, the method comprising (a) detecting ZNRF3 genomic loss in a prostate cancer sample from a subject; and (b) identifying the subject as being at high risk for metastatic prostate cancer. Also disclosed, in some aspects, is a method for prostate cancer prognosis, the method comprising (a) detecting reduced ZNRF3 expression in a prostate cancer sample from a subject relative to a control or reference sample; and (b) identifying the subject as being at high risk for metastatic prostate cancer. Further disclosed, in some aspects, is a method for prostate cancer prognosis, the method comprising (a) detecting increased ZNRF3 methylation in a prostate cancer sample from a subject relative to a control or reference sample; and (b) identifying the subject as being at high risk for metastatic prostate cancer. In some aspects, the method further comprises, prior to (b), detecting CCND1 genomic gain in the prostate cancer sample. In some aspects, the method further comprises administering to the subject an effective mount of a prostate cancer therapy.

In some aspects, the prostate cancer therapy comprises chemotherapy, hormone therapy, cryoablative therapy, hi-intensity ultrasound, photodynamic therapy, laser ablation, irreversible electroporation, radiotherapy, surgery, immunotherapy, or a combination thereof. In some aspects, the prostate cancer therapy comprises radiation and hormone therapy. In some aspects, the subject was further determined to have prostate cancer with CCND1 genomic gain. In some aspects, the subject was diagnosed with very low risk prostate cancer. In some aspects, the subject was diagnosed with low risk prostate cancer. In some aspects, the subject was diagnosed with intermediate favorable risk prostate cancer. In some aspects, the subject was diagnosed with high risk prostate cancer. In some aspects, the subject was diagnosed with very high risk prostate cancer. In some aspects, the subject was diagnosed with metastatic prostate cancer. In some aspects, the subject was diagnosed with intermediate unfavorable risk prostate cancer. In some aspects, the prostate cancer sample is a tissue sample. In some aspects, the prostate cancer sample is a blood sample. In some aspects, the prostate cancer sample is a plasma sample. In some aspects, the prostate cancer sample is a urine sample. In some aspects, the prostate cancer sample is a semen sample. In some aspects, the prostate cancer sample is a sample of circulating tumor cells. In some aspects, the prostate cancer sample is a cell-free nucleic acid sample.

Disclosed herein, in some aspects, is a method for diagnosing a subject with high risk or very high risk prostate cancer, the method comprising (a) detecting ZNRF3 genomic loss in a prostate cancer sample from the subject, (b) detecting reduced ZNRF3 expression in a prostate cancer sample from the subject, or (c) detecting increased ZNRF3 methylation in a prostate cancer sample from the subject. Further disclosed, in some aspects, is a method for treating a subject for prostate cancer, the method comprising administering an effective amount of a prostate cancer therapy to a subject (a) diagnosed with very low risk, low risk, or intermediate favorable risk prostate cancer and (b) determined to have prostate cancer with (i) ZNRF3 genomic loss, (ii) reduced ZNRF3 expression, and/or (iii) increased ZNRF3 methylation.

Disclosed herein, in some aspects, is a method for treating a subject for prostate cancer, the method comprising administering an effective amount of radiotherapy and hormone therapy to a subject (a) diagnosed with intermediate unfavorable risk prostate cancer and (b) determined to have prostate cancer with (i) ZNRF3 genomic loss, (ii) reduced ZNRF3 expression, and/or (iii) increased ZNRF3 methylation.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more.” “at least one,” and “one or more than one.”

The phrase “and/or” means “and” or “or”. To illustrate, A, B, and/or C includes: A alone, B alone, C alone, a combination of A and B, a combination of A and C, a combination of B and C. or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise.” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

“Individual, “subject,” and “patient” are used interchangeably and can refer to a human or non-human.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one aspect of the invention may apply to any other aspect of the invention. Furthermore, any composition of the invention may be used in any method of the invention, and any method of the invention may be used to produce or to utilize any composition of the invention. Any aspect discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an aspect set forth in the Examples are also aspects that may be implemented in the context of aspects discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIGS. 1A and 1B. Frequency Distribution of Genes Affected by Driver Mutations in Prostate Cancer. The full cohort (n=1,844) was split into localized (n=1,289) and metastatic (n=555) disease. Estimated proportion of patients in each cohort harboring each of 113 specific driver mutations. X-axis indicates the gene or genomic locus affected. Bar color indicates the type of driver mutation affecting that gene. Several genes are affected by multiple mutation types (e.g., TP53, PTEN, and others). Error bars represent 95% confidence intervals.

FIGS. 2A-2C. Prevalence of Driver Gene Mutations in Localized and Metastatic Prostate Cancer. FIG. 2A shows the proportion of tumours harboring each driver mutation in localized prostate cancer or mCRPC (‘Observed Δ Proportion’), as described. Dot size indicates—log₁₀ q-value; dot color indicates driver mutation type. Specific genes of interest are labeled. FIG. 2B shows comparison of driver gene mutation prevalence in localized disease and mCRPC. Differences in proportion of localized and metastatic cases harboring each driver mutation (‘Observed Δ Proportion’) were subtracted from the difference in proportions resulting from 100,000 simulations, per gene, per sample, where the probability of observing a mutation in a given sample was weighted by the global mutational burden (i.e. SNVs per Mb or PGA) in that sample (‘Expected Δ Proportion’) to generate the Adjusted Δ Proportion. A two-sided p-value was calculated as the proportion of simulated proportions that were as extreme or more extreme than the observed Δ proportion. Q-values were then derived using the False Discovery Rate method. FIG. 2C shows positive adjusted Δ proportion indicates higher than expected prevalence in mCRPC, while negative adjusted Δ proportion indicates lower than expected prevalence. Mutations are ordered from top to bottom by adjusted Δ proportion. Statistical significance was tested using adjusted Fisher's Exact tests with correction for multiple testing using the False Discovery Rate method. Mutations with q-value<0.05 were considered statistically significant. Error bars represent 95% confidence intervals.

FIGS. 3A-3D. ZNRF3 Genomic Loss is an Independent Prognostic Factor for Aggressive Localized Prostate Cancer. Biochemical relapse-free rate (FIG. 3A) and metastatic relapse-free rate (FIG. 3B; mRFR) in CPCG patients with tumour specimens with (blue) or without (red) genomic loss of ZNRF3 are shown. FIGS. 3C and 3D show forest plots of multivariable Cox proportional hazards analyses of ZNRF3 CNA status with clinical prognostic factors for biochemical relapse (FIG. 3C) and metastatic relapse (FIG. 3D).

FIGS. 4A-4D. Molecular and Clinical Correlates of ZNRF3 Genomic Loss. FIG. 4A shows patients in the CPCG cohort ordered from left to right by percentage of the genome altered by a copy number aberration (PGA). Patient age at diagnosis (years) is also shown. Blue bars correspond with patients harboring ZNRF3 genomic loss. Heatmaps below show clinico-molecular features (ZNRF3 Loss, BCR at any time, BCR within 30 months of treatment, Metastasis, ETS Fusion status, and IDC-P/CA histology; ‘present’ in blue, ‘absent’ in white, ‘not available’ in grey), clinical prognostic factors (Gleason Score, pre-treatment PSA, and clinical T category), and treatment type (image-guided radiotherapy; IGRT, blue or radical prostatectomy; RP, orange); FIG. 4B shows gene Set Enrichment Analysis of TCGA tumors harboring ZNRF3 loss. FIG. 4C shows metastatic relapse-free rate in CPCG patients, stratified by ZNRF3 genomic loss and CCND1 genomic gain. P-value from a log-rank test. FIG. 4D shows a forest plot of CPCG patients, stratified by ZNRF3 genomic loss, CCP/Prolaris score (continuous), clinical prognostic factors, and PGA. P-values from a Wald test.

FIG. 5. Data Availability for Localized Prostate Cancer Cases. Overlap of available samples for SNV, CNA, and SV data from 1,844 patients in the study cohort.

FIG. 6. Heatmap of Driver Gene Mutations in 1,884 Prostate Cancers. Each row represents an individual patient. Each column is a separate driver gene mutation. Blue and white represent the presence or absence of the specific mutation in the particular patient, respectively. Light brown indicates that this mutation type was not analyzed in the specific patient.

FIG. 7. Multivariable Cox Proportional Hazards Model of Prognostic, mCRPC-Enriched Driver Mutations. Association between risk of metastatic relapse in men with localized prostate cancer (CPCG; n=376) and CNAs in MYC, CCND1, PRKDC (gain), and ZNRF3, TP53, ETV5, and CDK12 (loss) were assessed in a multivariable Cox proportional hazards model including all four of these driver CNAs. ZNRF3, MYC, CCND1, and CDK12 remained independently prognostic on multivariable analysis.

FIGS. 8A-8C. Frequency of ZNRF3 loss in Localized Prostate Cancers, Stratified by Metastasis or Progression. Patients were stratified based on whether they experienced a metastatic relapse (FIG. 8A; CPCG cohort) or disease progression (FIG. 8B; TCGA cohort) or based on tumor grade (FIG. 8C). Blue indicates the proportion of patients who are ZNRF3 neutral; red indicates patients with ZNRF3 loss.

FIG. 9. ZNRF3 Loss is Associated with Reduced ZNRF3 RNA Abundance. ZNRF3 RNA abundance was assessed in four independent cohorts (CPCG, MSKCC, TCGA, and EOPC, as indicated) and stratified based on ZNRF3 loss. Left panels show density plots of log 2 ZNRF3 RNA abundance in each cohort. Right panels show log 2 ZNRF3 RNA abundance stratified by ZNRF3 loss. P-values are from a Mann-Whitney U test.

FIGS. 10A-10E. ZNRF3 RNA Abundance is Inversely Associated with Tumor ISUP Grade. Associations between diagnostic ISUP grade and ZNRF3 RNA abundance were assessed for four independent cohorts (FIG. 10A: CPCG, FIG. 10B: EOPC, FIG. 10C: TCGA, FIG. 10D: LTRI, FIG. 10E: MSKCC). P-value shown is from a one-way Analysis of Variance with Tukey post-hoc tests to assess between-groups significance, indicated as follows: ****-p<0.0001; ***-p<0.001; **-p<0.01; *-p<0.05.

FIGS. 11A-11B. ZNRF3 RNA Abundance is Inversely Associated with Risk of Metastatic Relapse. Multivariable Cox proportional hazards model of ZNRF3 RNA abundance (continuous) and clinical prognostic factors on metastatic relapse (FIG. 11A) or ZNRF3 RNA abundance and ZNRF3 genomic loss (FIG. 11B). P-values from a Wald test.

FIGS. 12A-12D. Validation of Association of Low ZNRF3 RNA Abundance and Poor Clinical Outcome in Localized Prostate Cancer. Low ZNRF3 RNA abundance was associated with risk of progression-free survival (FIG. 12A; TCGA), biochemical relapse in the EOPC (FIG. 12B) and LTRI (FIG. 12C) cohorts, and metastatic relapse in the LTRI cohort (FIG. 12D).

FIGS. 13A-13C. ZNRF3 Loss is Associated with Increased Genomic Instability. Patients in the CPCG (FIG. 13A), TCGA (FIG. 13B), or LTRI (FIG. 13C) cohorts were stratified by ZNRF3 copy number status. Adjusted PGA was calculated as the total number of bases affected by a CNA divided by the total number of bases in the genome, excluding chromosome 22 in both cases.

FIG. 14. ZNRF3 Loss is an Independent Prognostic Factor for Metastatic Relapse in Localized Prostate Cancer. Multivariable Cox proportional hazards model (metastatic relapse) of ZNRF3 loss with ISUP grade, pre-treatment PSA, clinical T-category, adjusted PGA, and the presence of intraductal carcinoma of the prostate or cribriform architecture (IDC-P/CA).

FIGS. 15A-15C. Associations Between ZNRF3 Loss and Cell Cycle Progression. Gene Set Enrichment Analysis of tumors harboring ZNRF3 loss in the EOPC cohort of localized prostate cancer (FIG. 15A) and the Abida cohort of mCRPC (FIG. 15B). FIG. 15C shows biochemical relapse-free survival in CPCG patients, stratified by ZNRF3 loss and CCND1 gain.

### DETAILED DESCRIPTION

The present disclosure is based, at least in part, on the discovery that ZNRF3 genomic loss, decreased RNA expression, and/or increased methylation in prostate cancer is prognostic for metastatic relapse and overall survival, and can be used to inform and guide treatment decisions for more effective prostate cancer treatment. Accordingly, aspects of the present disclosure are directed to methods for treating a subject determined to have ZNRF3 genomic loss, reduced ZNRF3 expression (e.g., as measured by RNA or protein levels), and/or increased ZNRF3 methylation in a prostate cancer sample. Further aspects are directed to methods for diagnosing a subject with high risk or very high risk prostate cancer comprising detecting ZNRF3 genomic loss, reduced ZNRF3 expression, and/or increased ZNRF3 methylation in a prostate cancer sample from the subject. The disclosed methods may comprise providing aggressive treatment in such a subject, for example treating such a subject with radiation and hormone therapy where a subject was previously diagnosed with very low risk, low risk, intermediate favorable risk, or intermediate unfavorable risk prostate cancer based on certain diagnostic methods (e.g., Gleason/ISUP grade, pre-treatment serum concentration of prostate-specific antigen (PSA), and/or clinical T category).

### I. Genetic Analysis, Diagnosis, and Treatment of Prostate Cancer

Aspects of the present disclosure include methods for analysis of genetic mutations, such as copy number variation (e.g., genomic loss, genomic gain), gene expression, and/or methylation status of one or more genes from a prostate cancer sample. As disclosed herein, such methods may be useful in, for example, diagnosis, prognosis, and/or treatment of a subject with prostate cancer.

In some aspects, disclosed are methods for analysis of copy number variation, gene expression, and/or methylation status of the gene ZNRF3. E3 ubiquitin-protein ligase ZNRF3 is an enzyme, encoded by ZNRF3, that functions as a negative regulator of Wnt signaling, among other roles. An example mRNA sequence encoded by ZNRF3 is provided by RefSeq number NM_001206998. An example protein sequence encoded by ZNRF3 is provided by RefSeq number NP_001193927.

In some aspects, disclosed are methods for analysis of copy number variation, gene expression, and/or methylation status of the gene CCND1. An example mRNA sequence encoded by CCND1 is provided by RefSeq number NM_053056. An example protein sequence encoded by CCND1 is provided by RefSeq number NP_444284.1.

Additional genes may be analyzed for copy number variation, gene expression, and/or methylation status as disclosed elsewhere herein. Example genes which may be analyzed include the genes listed in any of Tables 1-12. In some aspects, the present disclosure comprises detecting a mutation of Table 4 in a prostate cancer sample from a subject. Any 1, 2, 3, 4, 5, 6, 7,8, 9, 10, 11, 12, 13, 14, 15, 16, 17, 18, 19, 20, 21, 22, 23, or 24 of the mutations of Table 4 (in any combination) may be detected from a prostate cancer sample. For example, aspects of the present disclosure include detecting one or more of the following from a prostate cancer sample: genomic loss of BRCA2, genomic gain of CCND1, genomic loss of CDH1, genomic loss of CDK12, genomic los of CHD1, a structural variation of CHD1, an inversion of chr10:89 Mbp, an inter-chromosomal translocation of chr21:42 Mbp, an inversion of chr3:125 Mbp, genomic gain of ETV1, genomic gain of ETC5, a non-synonymous mutation of MSH2, genomic gain of MYC, genomic loss of NKX3-1, a structural variation of NKX3-1, genomic gain of PRKDC, genomic loss of PTEN, genomic los of RB1, a structural variation of RB1, a non-synonymous mutation of SPOP, genomic loss of TP53, genomic loss of ZBTB 16, genomic los of ZFHX3, and genomic loss of ZNRF3. Any combination of 1, 2, 3, 4, 5, 6, 7,8, 9, 10, 11, 12, 13, 14, 15, 16, 17, 18, 19, 20, 21, 22, 23, or 24 of the preceeding mutations may be detected from a prostate cancer sample. Further, a subject may be treated for pancreatic cancer, wherein the subject was determined to have one or more of the mutations of Table 4 in a prostate cancer sample. Accordingly, aspects of the present disclosure include treating a subject determined to have, from a prostate cancer sample from the subject, one or more of: genomic loss of BRCA2, genomic gain of CCND1, genomic loss of CDH1, genomic loss of CDK12, genomic los of CHD1, a structural variation of CHD1, an inversion of chr10:89 Mbp, an inter-chromosomal translocation of chr21:42 Mbp, an inversion of chr3:125 Mbp, genomic gain of ETV1, genomic gain of ETC5, a non-synonymous mutation of MSH2, genomic gain of MYC, genomic loss of NKX3-1, a structural variation of NKX3-1, genomic gain of PRKDC, genomic loss of PTEN, genomic los of RB1, a structural variation of RB1, a non-synonymous mutation of SPOP, genomic loss of TP53, genomic loss of ZBTB16, genomic los of ZFHX3, and genomic loss of ZNRF3. Any one or more of the preceeding mutations may be excluded from certain aspects of the disclosure.

Aspects of the present disclosure comprise detecting genomic loss of a gene (e.g., ZNRF3) in a sample, such as a cancer sample (e.g., prostate cancer sample). As used herein, a cancer sample having “genomic loss” of a gene describes a cancer sample from a subject which has a reduced amount of the gene relative to the normal amount of the gene in healthy, non-cancer cells from the individual. For example, where healthy cells from an individual have 2 copies of a gene per cell (e.g., an autosomal gene), detection of genomic loss of the gene in a cancer sample from the individual comprises detection of less than 2 copies of the gene per cell in the cancer sample. Where healthy cells from an individual have 2 copies of a gene per cell, detecting genomic loss of the gene may comprise detection of at most 1.99, 1.95, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, or exactly zero copies of the gene per cell, or any range or value derivable therein.

Aspects of the present disclosure comprise detecting genomic gain of a gene (e.g., CCND1) in a sample, such as a cancer sample (e.g., prostate cancer sample). As used herein, a cancer sample having “genomic gain” of a gene describes a cancer sample from a subject which has an increased amount of the gene relative to the normal amount of the gene in healthy, non-cancer cells from the individual. For example, where healthy cells from an individual have 2 copies of a gene per cell (e.g., an autosomal gene), detection of genomic gain of the gene in a cancer sample from the individual comprises detection of more than 2 copies of the gene per cell in the cancer sample. Where healthy cells from an individual have 2 copies of a gene per cell, detecting genomic gain of the gene may comprise detection of at least 2.01, 2.05, 2.1, 2.2, 2.3, 2.4, 2.5, 2.6, 2.7, 2.8, 2.9, 3, 4, 5, or 6 copies of the gene per cell, or more, or any range or value derivable therein.

Various means for detection of genomic loss and genomic gain are recognized in the art and contemplated herein, including, for example, methods described in Quigley, D. A. et al. Genomic Hallmarks and Structural Variation in Metastatic Prostate Cancer. Cell 174, 758-769.e9 (2018), incorporated herein by reference in its entirety.

Aspects of the disclosed methods comprise detecting a reduced expression level of a gene (e.g., ZNRF3), for example as measured by mRNA and/or protein expression. As used herein, a cancer sample having “reduced expression” of a gene describes a cancer sample from a subject which has reduced expression (e.g., RNA or protein level) of the gene relative to a control or reference. In some aspects, the control or reference is an average expression level of the gene from a plurality of other cancer samples. For example, a prostate cancer sample from one individual has reduced expression of a gene where the expression level of the gene is lower than an average expression level of the gene in a plurality of other prostate cancer samples. Detecting a reduced expression level may comprise detecting an expression level that is at least, at most, or about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9%, or any range or value derivable therein, lower than an expression level of the gene in a control or reference. In some aspects, the expression level is reduced by at least 50%, 60%, 70%, 80%, or 90%. In some aspects, the expression level is reduced by at least 50%. In some aspects, the expression level is reduced by at least 90%.

In some aspects, the disclosed methods comprise detecting an increased expression level of a gene, for example as measured by mRNA and/or protein expression. As used herein, a cancer sample having “increased expression” of a gene describes a cancer sample from a subject having an increased expression (e.g., RNA or protein level) of the gene relative to a control or reference. In some aspects, the control or reference is an average expression level of the gene from a plurality of other cancer samples. For example, a prostate cancer sample from one individual has increased expression of gene where the expression of the gene is higher than an average expression of the gene in a plurality of other prostate cancer samples. Detecting an increased expression level may comprise detecting an expression level that is at least, at most, or about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9%, 100%, 110%, 120%, 130%, 140%, 150%, 200%, 300%, 400%, or 500%, or any range or value derivable therein, higher than an expression level of the gene in a control or reference.

Aspects of the disclosed methods comprise detecting a reduced methylation level (also “reduced methylation”) of a gene, for example as measured by methylation-specific sequencing such as bisulfite sequencing. As used herein, a cancer sample having “reduced methylation” of a gene describes a cancer sample from a subject having reduced methylation of the gene (including methylation of exons, introns, promoters, enhancers, or other regulatory regions of the gene) relative to a control or reference. In some aspects, the control or reference is an average methylation of the gene from a plurality of other cancer samples. For example, a prostate cancer sample from one individual has reduced methylation of gene where the methylation level of the gene is lower than an average methylation level of the gene in a plurality of other prostate cancer samples. Detecting a reduced methylation level may comprise detecting a methylation level that is at least, at most, or about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9%, or any range or value derivable therein, lower than a methylation level of the gene in a control or reference.

Aspects of the disclosed methods comprise detecting an increased methylation level (also “increased methylation”) of a gene, for example as measured by methylation-specific sequencing such as bisulfite sequencing. As used herein, a cancer sample having “increased methylation” of a gene describes a cancer sample from a subject having increased methylation of the gene (including methylation of exons, introns, promoters, enhancers, or other regulatory regions of the gene) relative to a control or reference. In some aspects, the control or reference is an average methylation of the gene from a plurality of other cancer samples. For example, a prostate cancer sample from one individual has an increased methylation of gene where the methylation level of the gene is higher than the average methylation level of the gene in a plurality of other prostate cancer samples. Detecting an increased methylation level may comprise detecting a methylation level that is at least, at most, or about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9%, 100%, 110%, 120%, 130%, 140%, 150%, 200%, 300%, 400%, or 500%, or any range or value derivable therein, higher than a methylation level of the gene in a control or reference.

As used herein, a “cancer sample” describes any sample comprising a cancer cell or one or more components from a cancer cell (e.g., nucleic acid such as DNA or RNA, protein, etc.). In some aspects, a cancer sample is a tissue sample. In some aspects, a cancer sample is a liquid sample (e.g., blood, plasma, urine). In some aspects, a cancer sample is a sample comprising cell-free tumor nucleic acid (e.g., cell-free tumor DNA, cell-free tumor RNA). In some aspects, a cancer sample is obtained from a biopsy. A cancer sample may be a sample of any type of cancer. In some aspects, a cancer sample of the disclosure is a prostate cancer sample.

Aspects of the present disclosure comprise methods for diagnosis and/or prognosis of prostate cancer based on genomic analysis. In certain aspects, disclosed are methods for diagnosis of a subject with prostate cancer. The disclosed methods may comprise diagnosis of a subject with prostate cancer of a particular risk group (or “risk level”). Prostate cancer risk groups include, for example, very low risk, low risk, intermediate favorable risk (also “favorable intermediate risk”), intermediate unfavorable risk (also “unfavorable intermediate risk”), high risk, and very high risk. Prostate cancer risk groups include, for example, those provided by the National Comprehensive Cancer Network Clinical Practice Guidelines in Oncology (NCCN Guidelines®) for prostate cancer.

In some aspects, disclosed are methods for diagnosing a subject as having high risk or very high risk prostate cancer by detecting genomic loss, reduced expression, and/or increased methylation of ZNRF3 from a prostate cancer sample from the subject. In some aspects, such a subject was previously diagnosed with very low risk, low risk, intermediate favorable risk, or intermediate unfavorable risk prostate cancer. For example, in certain aspects, disclosed is a method comprising obtaining a prostate cancer sample from a subject previously diagnosed with intermediate unfavorable risk prostate cancer (e.g., via Gleason/ISUP score, pre-treatment serum concentration of prostate-specific antigen (PSA), and/or clinical T category), detecting genomic loss of ZNRF3 in the prostate cancer sample, and diagnosing the subject with high risk or very high risk prostate cancer. In such aspects, the method may further comprise treating the subject with aggressive prostate cancer therapy (e.g., radiotherapy and hormone therapy). In another example, in certain aspects, disclosed is a method comprising obtaining a prostate cancer sample from a subject previously diagnosed with intermediate favorable risk prostate cancer (e.g., via Gleason/ISUP score, pre-treatment serum concentration of prostate-specific antigen (PSA), and/or clinical T category), detecting genomic loss of ZNRF3 in the prostate cancer sample, and diagnosing the subject with high risk or very high risk prostate cancer. In such aspects, methods of the disclosure may further comprise treating the subject with a prostate cancer therapy (e.g., surgery, radiotherapy). Accordingly, aspects of the disclosure are useful for determining and providing the most effective treatment for a subject with prostate cancer.

### II. Therapeutic Methods

Aspects of the present disclosure comprise therapeutic methods and compositions for use thereof. Compositions of the disclosure may be used for in vivo, in vitro, and/or ex vivo administration.

### A. Cancer Therapy

In some aspects, the disclosed methods comprise administering a cancer therapy to a subject or patient. The cancer therapy may be chosen based on an expression level measurements, alone or in combination with the clinical risk score calculated for the subject. The cancer therapy may be chosen based on a genotype of a subject. The cancer therapy may be chosen based on the presence or absence of one or more polymorphisms in a subject. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pincaloma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some aspects, the cancer is aggressive cancer. In some aspects, the cancer is Stage I cancer. In some aspects, the cancer is Stage II cancer (e.g., IIA, IIB, IIC). In some aspects, the cancer is Stage III cancer (e.g., IIIA, IIIB, IIIC). In some aspects, the cancer is Stage IV cancer (e.g., IVA, IVB).

In some aspects, disclosed are methods for treating cancer originating from the prostate. In some aspects, the cancer is prostate cancer. In some aspects, the cancer is a recurrent cancer. In some aspects, the cancer is an immunotherapy-resistant cancer.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

Biomarkers, like copy number variations or expression differences in particular can, in some cases, predict the efficacy of certain therapeutic regimens and can be used to identify patients who will receive benefit from a particular therapy.

### B. Cancer Immunotherapy

In some aspects, the methods comprise administration of a cancer immunotherapy. Cancer immunotherapy (sometimes called immuno-oncology, abbreviated IO) is the use of the immune system to treat cancer. Immunotherapies can, in some cases, be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and certain examples are described below.

### 1. Checkpoint Inhibitors and Combination Treatment

Aspects of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy.” “ICB,” or “CBI”), refers to cancer therapy comprising providing one or more immune checkpoint inhibitors to a subject suffering from or suspected of having cancer.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC. Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the Vu region of nivolumab, pembrolizumab, or pidilizumab, and the CDR1, CDR2 and CDR3 domains of the V_{L} region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the V_{H} region of tremelimumab or ipilimumab, and the CDR1, CDR2 and CDR3 domains of the V_{L} region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some aspects, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4 (Supp. 1): P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some aspects, the inhibitor comprises the heavy and light chain CDRs or VRs of an anti-LAG3 antibody. Accordingly, in one aspect, the inhibitor comprises the CDR1, CDR2, and CDR3 domains of the V_{H} region of an anti-LAG3 antibody, and the CDR1, CDR2 and CDR3 domains of the V_{L} region of an anti-LAG3 antibody. In another aspect, the antibody has at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+ Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+ Th1-specific cell surface protein that regulates macrophage activation. Inhibitors of the disclosure may block one or more functions of TIM-3 activity.

In some aspects, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some aspects, the inhibitor comprises the heavy and light chain CDRs or VRs of an anti-TIM-3 antibody. Accordingly, in one aspect, the inhibitor comprises the CDR1, CDR2, and CDR3 domains of the V_{H} region of an anti-TIM-3 antibody, and the CDR1, CDR2 and CDR3 domains of the V_{L} region of an anti-TIM-3 antibody. In another aspect, the antibody has at least about 70, 75, 80, 85, 90, 95, 97, or 99% (or any derivable range or value therein) variable region amino acid sequence identity with the above-mentioned antibodies.

### 2. Activator of Co-Stimulatory Molecules

In some aspects, the immunotherapy comprises an activator (also “agonist”) of a co-stimulatory molecule. In some aspects, the agonist comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include activating antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell, natural killer (NK) cell, or other immune cell. The receptors are called chimeric because they are fused of parts from different sources. CAR-T cell therapy refers to a treatment that uses such transformed cells for cancer therapy, where the transformed cells are T cells. Similar therapies include, for example, CAR-NK cell therapy, which uses transformed NK cells.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

5. Cytokine therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### C. Oncolytic Virus

In some aspects, the cancer therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

### D. Chemotherapies

In some aspects, a therapy of the present disclosure comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-a), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection.

Other suitable chemotherapeutic agents include antimicrotubule agents, e.g., Paclitaxel (“Taxol”) and doxorubicin hydrochloride (“doxorubicin”). Doxorubicin is absorbed poorly and is preferably administered intravenously. In certain aspects, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week.

Nitrogen mustards are another suitable chemotherapeutic agent useful in the methods of the disclosure. A nitrogen mustard may include, but is not limited to, mechlorethamine (HN2), cyclophosphamide and/or: ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred in certain cases. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

The amount of the chemotherapeutic agent delivered to a patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### E. Hormone Therapy

In some aspects, a cancer therapy of the present disclosure is a hormone therapy. In particular aspects, a prostate cancer therapy comprises hormone therapy. Various hormone therapies are known in the art and contemplated herein. Examples of hormone therapies include, but are not limited to, luteinizing hormone-releasing hormone (LHRH) analogs, LHRH antagonists, androgen receptor antagonists, and androgen synthesis inhibitors.

### F. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### G. Radiotherapy

In some aspects, a cancer therapy (e.g., prostate cancer therapy) comprises radiation, such as ionizing radiation (IR). As used herein, “ionizing radiation” means radiation comprising particles or photons that have sufficient energy or can produce sufficient energy via nuclear interactions to produce ionization (gain or loss of electrons). An example ionizing radiation is an x-radiation. Various means for delivering x-radiation to a target tissue or cell are well known in the art and.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of IR may be presented as a total dose of IR, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### H. Additional Cancer Therapies

Therapeutic methods disclosed herein may comprise one or more additional cancer therapies. A cancer therapy (e.g., prostate cancer therapy) of the disclosure may comprise, for example, cryoablative therapy, high-intensity ultrasound (also “high-intensity focused ultrasound”), photodynamic therapy, laser ablation, and/or irreversible electroporation. A cancer therapy of the disclosure may comprise 1, 2, 3, 4, 5, or more distinct therapeutic methods.

### III. Sample Preparation

In certain aspects, methods involve obtaining a sample (also “biological sample”) from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain aspects the sample is obtained from a biopsy from prostate tissue by any of the biopsy methods previously mentioned. In other aspects the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, serum, plasma, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include, but is not limited to, tissue, cells, or biological material from cells or derived from cells of a subject. The biological sample may be a heterogeneous or homogeneous population of cells or tissues. Alternatively, the biological sample may be a cell-free sample, for example serum or plasma. The biological sample may be obtained using any method known to the art that can provide a sample suitable for the analytical methods described herein. The sample may be obtained by non-invasive methods including but not limited to: scraping of the skin or cervix, swabbing of the check, blood collection, saliva collection, urine collection, feces collection, or collection of menses, tears, or semen. In some aspects, a sample comprises nucleic acids from the subject. In some aspects, a sample comprises nucleic acids from one or more cancer cells from a subject. In some aspects, a sample comprises tumor DNA (i.e., DNA from one or more cancer cells). In some aspects, a sample comprises tumor RNA (i.e., RNA from one or more cancer cells). In some aspects, a sample is a cell free sample. In some aspects, a sample comprises cell free DNA (cfDNA). In some aspects, the sample is a blood sample. In some aspects, the sample is a saliva sample. In some aspects, the sample is a urine sample.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple prostate samples may be obtained for diagnosis by the methods described herein. In other cases, multiple samples, such as one or more samples from one tissue type (for example prostate) and one or more samples from another specimen (for example serum) may be obtained for diagnosis by the methods. In some cases, multiple samples such as one or more samples from one tissue type (e.g. prostate) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a semen sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one aspect, the sample is a fine needle aspirate of a prostate or a suspected prostate tumor or neoplasm. In some cases, the fine needle aspirate sampling procedure may be guided by the use of an ultrasound, X-ray, or other imaging device.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### IV. Assay Methods

### A. Detection of Methylated DNA

Aspects of the methods include assaying nucleic acids to determine expression levels and/or methylation levels of nucleic acids. Assays for the detection of methylated DNA are known in the art. Example methods are described herein.

### 1. HPLC-UV

The technique of HPLC-UV (high performance liquid chromatography-ultraviolet), developed by Kuo and colleagues in 1980 (described further in Kuo K. C. et al., Nucleic Acids Res. 1980; 8:4763-4776, which is herein incorporated by reference) can be used to quantify the amount of deoxycytidine (dC) and methylated cytosines (5 mC) present in a hydrolysed DNA sample. The method includes hydrolyzing the DNA into its constituent nucleoside bases, the 5 mC and dC bases are separated chromatographically and, then, the fractions are measured. Then, the 5 mC/dC ratio can be calculated for each sample, and this can be compared between the experimental and control samples.

### 2. LC-MS/MS

Liquid chromatography coupled with tandem mass spectrometry (LC-MS/MS) is an high-sensitivity approach to HPLC-UV, which requires much smaller quantities of the hydrolysed DNA sample. In the case of mammalian DNA, of which ˜2%-5% of all cytosine residues are methylated, LC-MS/MS has been validated for detecting levels of methylation levels ranging from 0.05%-10%, and it can confidently detect differences between samples as small as ˜0.25% of the total cytosine residues, which corresponds to ˜5% differences in global DNA methylation. The procedure routinely requires 50-100 ng of DNA sample, although much smaller amounts (as low as 5 ng) have been successfully profiled. Another major benefit of this method is that it is not adversely affected by poor-quality DNA (e.g., DNA derived from FFPE samples).

### 3. ELISA-Based Methods

There are several commercially available kits, all enzyme-linked immunosorbent assay (ELISA) based, that enable the quick assessment of DNA methylation status. These assays include Global DNA Methylation ELISA, available from Cell Biolabs; Imprint Methylated DNA Quantification kit (sandwich ELISA), available from Sigma-Aldrich; EpiSeeker methylated DNA Quantification Kit, available from abcam; Global DNA Methylation Assay-LINE-1, available from Active Motif; 5-mC DNA ELISA Kit, available from Zymo Research; MethylFlash Methylated DNA5-mC Quantification Kit and MethylFlash Methylated DNA5-mC Quantification Kit, available from Epigentek.

Briefly, the DNA sample is captured on an ELISA plate, and the methylated cytosines are detected through sequential incubations steps with: (1) a primary antibody raised against 5 Mc; (2) a labelled secondary antibody; and then (3) colorimetric/fluorometric detection reagents.

The Global DNA Methylation Assay—LINE-1 specifically determines the methylation levels of LINE-1 (long interspersed nuclear elements-1) retrotransposons, of which ˜17% of the human genome is composed. These are well established as a surrogate for global DNA methylation. Briefly, fragmented DNA is hybridized to biotinylated LINE-1 probes, which are then subsequently immobilized to a streptavidin-coated plate. Following washing and blocking steps, methylated cytosines are quantified using an anti-5 mC antibody, HRP-conjugated secondary antibody and chemiluminescent detection reagents. Samples are quantified against a standard curve generated from standards with known LINE-1 methylation levels. The manufacturers claim the assay can detect DNA methylation levels as low as 0.5%. Thus, by analysing a fraction of the genome, it is possible to achieve better accuracy in quantification.

### 4. LINE-1 Pyrosequencing

Levels of LINE-1 methylation can alternatively be assessed by another method that involves the bisulfite conversion of DNA, followed by the PCR amplification of LINE-1 conservative sequences. The methylation status of the amplified fragments is then quantified by pyrosequencing, which is able to resolve differences between DNA samples as small as ˜5%. Even though the technique assesses LINE-1 elements and therefore relatively few CpG sites, this has been shown to reflect global DNA methylation changes very well. The method is particularly well suited for high throughput analysis of cancer samples, where hypomethylation is very often associated with poor prognosis. This method is particularly suitable for human DNA, but there are also versions adapted to rat and mouse genomes.

### 5. AFLP and RFLP

Detection of fragments that are differentially methylated could be achieved by traditional PCR-based amplification fragment length polymorphism (AFLP), restriction fragment length polymorphism (RFLP) or protocols that employ a combination of both.

### 6. LUMA

The LUMA (luminometric methylation assay) technique utilizes a combination of two DNA restriction digest reactions performed in parallel and subsequent pyrosequencing reactions to fill-in the protruding ends of the digested DNA strands. One digestion reaction is performed with the CpG methylation-sensitive enzyme HpaII; while the parallel reaction uses the methylation-insensitive enzyme MspI, which will cut at all CCGG sites. The enzyme EcoRI is included in both reactions as an internal control. Both MspI and HpaII generate 5′-CG overhangs after DNA cleavage, whereas EcoRI produces 5′-AATT overhangs, which are then filled in with the subsequent pyrosequencing-based extension assay. Essentially, the measured light signal calculated as the HpaII/MspI ratio is proportional to the amount of unmethylated DNA present in the sample. As the sequence of nucleotides that are added in pyrosequencing reaction is known, the specificity of the method is very high and the variability is low, which is essential for the detection of small changes in global methylation. LUMA requires only a relatively small amount of DNA (250-500 ng), demonstrates little variability and has the benefit of an internal control to account for variability in the amount of DNA input.

### 7. Bisulfite Sequencing

The bisulfite treatment of DNA mediates the deamination of cytosine into uracil, and these converted residues will be read as thymine, as determined by PCR-amplification and subsequent Sanger sequencing analysis. However, 5 mC residues are resistant to this conversion and, so, will remain read as cytosine. Thus, comparing the Sanger sequencing read from an untreated DNA sample to the same sample following bisulfite treatment enables the detection of the methylated cytosines. With the advent of next-generation sequencing (NGS) technology, this approach can be extended to DNA methylation analysis across an entire genome. To ensure complete conversion of non-methylated cytosines, controls may be incorporated for bisulfite reactions.

Whole genome bisulfite sequencing (WGBS) is similar to whole genome sequencing, except for the additional step of bisulfite conversion. Sequencing of the 5 mC-enriched fraction of the genome is not only a less expensive approach, but it also allows one to increase the sequencing coverage and, therefore, precision in revealing differentially-methylated regions. Sequencing could be done using any existing NGS platform; Illumina and Life Technologies both offer kits for such analysis.

Bisulfite sequencing methods include reduced representation bisulfite sequencing (RRBS), where only a fraction of the genome is sequenced. In RRBS, enrichment of CpG-rich regions is achieved by isolation of short fragments after MspI digestion that recognizes CCGG sites (and it cut both methylated and unmethylated sites). It ensures isolation of ˜85% of CpG islands in the human genome. Then, the same bisulfite conversion and library preparation is performed as for WGBS. The RRBS procedure normally requires ˜100 ng-1 μg of DNA.

Various bisulfite sequencing methods are known in the art and contemplated herein.

8. Methods that Exclude Bisulfite Conversion

In some aspects, direct detection of modified bases without bisulfite conversion may be used to detect methylation. Pacific Biosciences company has developed a way to detect methylated bases directly by monitoring the kinetics of polymerase during single molecule sequencing and offers a commercial product for such sequencing (further described in Flusberg B. A., et al., Nat. Methods. 2010; 7:461-465, which is herein incorporated by reference). Other methods include nanopore-based single-molecule real-time sequencing technology (SMRT), which is able to detect modified bases directly (described in Laszlo A. H. et al., Proc. Natl. Acad. Sci. USA. 2013 and Schreiber J., et al., Proc. Natl. Acad. Sci. USA. 2013, which are herein incorporated by reference).

### 9. Array or Bead Hybridization

Methylated DNA fractions of the genome, usually obtained by immunoprecipitation, could be used for hybridization with microarrays. Currently available examples of such arrays include: the Human CpG Island Microarray Kit (Agilent), the GeneChip Human Promoter 1.0R Array and the GeneChip Human Tiling 2.0R Array Set (Affymetrix).

The search for differentially-methylated regions using bisulfite-converted DNA could be done with the use of different techniques. Some of them are easier to perform and analyse than others, because only a fraction of the genome is used. The most pronounced functional effect of DNA methylation occurs within gene promoter regions, enhancer regulatory elements and 3′ untranslated regions (3′UTRs). Assays that focus on these specific regions, such as the Infinium HumanMethylation450 Bead Chip array by Illumina, can be used. The arrays can be used to detect methylation status of genes, including miRNA promoters, 5′ UTR. 3′ UTR, coding regions (˜17 CpG per gene) and island shores (regions ˜2 kb upstream of the CpG islands).

Briefly, bisulfite-treated genomic DNA is mixed with assay oligos, one of which is complimentary to uracil (converted from original unmethylated cytosine), and another is complimentary to the cytosine of the methylated (and therefore protected from conversion) site. Following hybridization, primers are extended and ligated to locus-specific oligos to create a template for universal PCR. Finally, labelled PCR primers are used to create detectable products that are immobilized to bar-coded beads, and the signal is measured. The ratio between two types of beads for each locus (individual CpG) is an indicator of its methylation level.

It is possible to purchase kits that utilize the extension of methylation-specific primers for validation studies. In the VeraCode Methylation assay from Illumina, 96 or 384 user-specified CpG loci are analysed with the GoldenGate Assay for Methylation. Differently from the BeadChip assay, the VeraCode assay requires the BeadXpress Reader for scanning.

### 10. Methyl-Sensitive Cut Counting: Endonuclease Digestion Followed by Sequencing

As an alternative to sequencing a substantial amount of methylated (or unmethylated) DNA, one could generate snippets from these regions and map them back to the genome after sequencing. Moreover, coverage in NGS could be good enough to quantify the methylation level for particular loci. The technique of serial analysis of gene expression (SAGE) has been adapted for this purpose and is known as methylation-specific digital karyotyping, as well as a similar technique, called methyl-sensitive cut counting (MSCC).

In summary, in all of these methods, methylation-sensitive endonuclease(s), e.g., HpaII is used for initial digestion of genomic DNA in unmethylated sites followed by adaptor ligation that contains the site for another digestion enzyme that is cut outside of its recognized site, e.g., EcoP15I or MmeI. These ways, small fragments are generated that are located in close proximity to the original HpaII site. Then, NGS and mapping to the genome are performed. The number of reads for each HpaII site correlates with its methylation level.

Recently, a number of restriction enzymes have been discovered that use methylated DNA as a substrate (methylation-dependent endonucleases). Most of them were discovered and are sold by SibEnzyme: BisI, BlsI, GlaI, GluI, KroI, MteI, PcsI, PkrI. The unique ability of these enzymes to cut only methylated sites has been utilized in the method that achieved selective amplification of methylated DNA. Three methylation-dependent endonucleases that are available from New England Biolabs (FspEI, MspJI and LpnPI) are type IIS enzymes that cut outside of the recognition site and, therefore, are able to generate snippets of 32 bp around the fully-methylated recognition site that contains CpG. These short fragments could be sequences and aligned to the reference genome. The number of reads obtained for each specific 32-bp fragment could be an indicator of its methylation level. Similarly, short fragments could be generated from methylated CpG islands with Escherichia coli's methyl-specific endonuclease McrBC, which cuts DNA between two half-sites of (G/A) mC that are lying within 50 bp-3000 bp from each other. This is a very useful tool for isolation of methylated CpG islands that again can be combined with NGS. Being bisulfite-free, these three approaches have a great potential for quick whole genome methylome profiling.

### B. Sequencing

DNA, including bisulfite-converted DNA could be used for the amplification of the region of interest followed by sequencing. The resulting PCR products could be cloned and sequenced. Accordingly, aspects of the disclosure may include sequencing nucleic acids to detect methylation of nucleic acids and/or biomarkers. In some aspects, the methods of the disclosure include a sequencing method. In some aspects, the methods of the disclosure include measuring an expression level of one or more genes using a sequencing method.

Example sequencing methods include those described below.

### 1. Massively Parallel Signature Sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the Illumina HiSeq2000, HiSeq2500 and MiSeq systems are based on MPSS.

### 2. Polony Sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing.

### 3. 454 Pyrosequencing.

A parallelized version of pyrosequencing was developed by 454 Life Sciences. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLID on the other.

### 4. Illumina (Solexa) Sequencing.

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massivelly parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera. Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony).

### 5. SOLID Sequencing.

SOLID technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

### 6. Ion Torrent Semiconductor Sequencing.

Ion Torrent Systems Inc. developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

### 7. DNA Nanoball Sequencing.

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects.

### 8. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

### 9. Single Molecule Real Time (SMRT) Sequencing.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.

### C. Additional Assay Methods

In some aspects, methods involve amplifying and/or sequencing one or more target genomic regions using at least one pair of primers specific to the target genomic regions. In certain aspects, the primers are heptamers. In other aspects, enzymes are added such as primases or primase/polymerase combination enzyme to the amplification step to synthesize primers.

In some aspects, arrays can be used to detect nucleic acids of the disclosure. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

In addition to the use of arrays and microarrays, it is contemplated that a number of difference assays could be employed to analyze nucleic acids. Such assays include, but are not limited to, nucleic amplification, polymerase chain reaction, quantitative PCR, RT-PCR, in situ hybridization, digital PCR, ddPCR (droplet digital PCR), nCounter (nanoString), BEAMing (Beads, Emulsions, Amplifications, and Magnetics) (Inostics), ARMS (Amplification Refractory Mutation Systems), RNA-Seq. TAm-Seg (Tagged-Amplicon deep sequencing), PAP (Pyrophosphorolysis-activation polymerization), next generation RNA sequencing, northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

Amplification primers or hybridization probes can be prepared to be complementary to a genomic region, biomarker, probe, or oligo described herein. The term “primer” or “probe” as used herein, is meant to encompass any nucleic acid that is capable of priming the synthesis of a nascent nucleic acid in a template-dependent process and/or pairing with a single strand of an oligo of the disclosure, or portion thereof. Typically, primers are oligonucleotides from ten to twenty and/or thirty nucleic acids in length, but longer sequences can be employed. Primers may be provided in double-stranded and/or single-stranded form, although the single-stranded form is preferred.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one aspect, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In one aspect, quantitative RT-PCR (such as TaqMan, ABI) is used for detecting and comparing the levels or abundance of nucleic acids in samples. The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable DNAs may be normalized to some independent standard/control, which may be based on either internally existing DNA species or externally introduced DNA species. The abundance of a particular DNA species may also be determined relative to the average abundance of all DNA species in the sample.

In one aspect, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in some samples is that they are of variable quantity and/or quality. This problem can be overcome if the RT-PCR is performed as a relative quantitative RT-PCR with an internal standard in which the internal standard is an amplifiable DNA fragment that is similar or larger than the target DNA fragment and in which the abundance of the DNA representing the internal standard is roughly 5-100 fold higher than the DNA representing the target nucleic acid region.

In another aspect, the relative quantitative RT-PCR uses an external standard protocol. Under this protocol, the PCR products are sampled in the linear portion of their amplification curves. The number of PCR cycles that are optimal for sampling can be empirically determined for each target DNA fragment. In addition, the nucleic acids isolated from the various samples can be normalized for equal concentrations of amplifiable DNAs.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some aspects, the density may be or may be at least 50, 100, 200, 300, 400, 500 or more probes/cm2 (or any range derivable therein).

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al. 1991). It is contemplated that this technology may be used in conjunction with evaluating the expression level of one or more cancer biomarkers (e.g., ZNRF3) with respect to diagnostic, prognostic, and treatment methods.

Certain aspects may involve the use of arrays or data generated from an array. Data may be readily available. Moreover, an array may be prepared in order to generate data that may then be used in correlation studies.

### V. Administration of Therapeutic Compositions

The therapy provided herein may comprise administration of a combination of therapeutic agents, such as a first cancer therapy (e.g., radiotherapy) and a second cancer therapy (e.g., hormone therapy). The therapies may be administered in any suitable manner known in the art. For example, the first and second cancer treatment may be administered sequentially (at different times) or concurrently (at the same time).

In some aspects, the first cancer therapy and the second cancer therapy are administered substantially simultaneously. In some aspects, the first cancer therapy and the second cancer therapy are administered sequentially. In some aspects, the first cancer therapy, the second cancer therapy, and a third therapy are administered sequentially. In some aspects, the first cancer therapy is administered before administering the second cancer therapy. In some aspects, the first cancer therapy is administered after administering the second cancer therapy.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose (also “effective amount” or “therapeutically effective amount”) is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 M; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 UM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### VI. Kits

Certain aspects of the present invention also concern kits containing compositions of the disclosure or compositions to implement methods of the disclosure. In some aspects, kits can be used to evaluate one or more biomarkers. In some aspects, kits can be used to detect, for example, genomic loss, reduced expression, or increased methylation of a gene (e.g., ZNRF3, CCND1). In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit aspects. In addition, a kit may include a sample that is a negative or positive control for copy number or expression of one or more biomarkers (e.g., ZNRF3, CCND1).

Any aspect of the disclosure involving specific biomarker by name is contemplated also to cover aspects involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

### VII. Detecting a Genetic Signature

Particular aspects concern the methods of detecting a genetic signature in an individual. In some aspects, the method for detecting the genetic signature may include selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

### A. Single Nucleotide Polymorphism (SNP) Detection

Certain aspects of the disclosure concern methods of detecting a SNP in an individual. One may employ any of the known general methods for detecting SNPs for detecting the particular SNP in this disclosure, for example. Such methods include, but are not limited to, selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting. DNA mismatch binding protein analysis, surveyor nuclease assay, sequencing, or a combination thereof.

In some aspects of the disclosure, the method used to detect the SNP comprises sequencing nucleic acid material from the individual and/or using selective oligonucleotide probes. Sequencing the nucleic acid material from the individual may involve obtaining the nucleic acid material from the individual in the form of genomic DNA, complementary DNA that is reverse transcribed from RNA, or RNA, for example. Any standard sequencing technique may be employed, including Sanger sequencing, chain extension sequencing, Maxam-Gilbert sequencing, shotgun sequencing, bridge PCR sequencing, high-throughput methods for sequencing, next generation sequencing, RNA sequencing, or a combination thereof. After sequencing the nucleic acid from the individual, one may utilize any data processing software or technique to determine which particular nucleotide is present in the individual at the particular SNP.

In some aspects, the nucleotide at the particular SNP is detected by selective oligonucleotide probes. The probes may be used on nucleic acid material from the individual, including genomic DNA, complementary DNA that is reverse transcribed from RNA, or RNA, for example. Selective oligonucleotide probes preferentially bind to a complementary strand based on the particular nucleotide present at the SNP. For example, one selective oligonucleotide probe binds to a complementary strand that has an A nucleotide at the SNP on the coding strand but not a G nucleotide at the SNP on the coding strand, while a different selective oligonucleotide probe binds to a complementary strand that has a G nucleotide at the SNP on the coding strand but not an A nucleotide at the SNP on the coding strand. Similar methods could be used to design a probe that selectively binds to the coding strand that has a C or a T nucleotide, but not both, at the SNP. Thus, any method to determine binding of one selective oligonucleotide probe over another selective oligonucleotide probe could be used to determine the nucleotide present at the SNP.

One method for detecting SNPs using oligonucleotide probes comprises the steps of analyzing the quality and measuring quantity of the nucleic acid material by a spectrophotometer and/or a gel electrophoresis assay; processing the nucleic acid material into a reaction mixture with at least one selective oligonucleotide probe, PCR primers, and a mixture with components needed to perform a quantitative PCR (qPCR), which could comprise a polymerase, deoxynucleotides, and a suitable buffer for the reaction; and cycling the processed reaction mixture while monitoring the reaction. In one aspect of the method, the polymerase used for the qPCR will encounter the selective oligonucleotide probe binding to the strand being amplified and, using endonuclease activity, degrade the selective oligonucleotide probe. The detection of the degraded probe determines if the probe was binding to the amplified strand.

Another method for determining binding of the selective oligonucleotide probe to a particular nucleotide comprises using the selective oligonucleotide probe as a PCR primer, wherein the selective oligonucleotide probe binds preferentially to a particular nucleotide at the SNP position. In some aspects, the probe is generally designed so the 3′ end of the probe pairs with the SNP. Thus, if the probe has the correct complementary base to pair with the particular nucleotide at the SNP, the probe will be extended during the amplification step of the PCR. For example, if there is a T nucleotide at the 3′ position of the probe and there is an A nucleotide at the SNP position, the probe will bind to the SNP and be extended during the amplification step of the PCR. However, if the same probe is used (with a T at the 3′ end) and there is a G nucleotide at the SNP position, the probe will not fully bind and will not be extended during the amplification step of the PCR.

In some aspects, the SNP position is not at the terminal end of the PCR primer, but rather located within the PCR primer. The PCR primer should be of sufficient length and homology in that the PCR primer can selectively bind to one variant, for example the SNP having an A nucleotide, but not bind to another variant, for example the SNP having a G nucleotide. The PCR primer may also be designed to selectively bind particularly to the SNP having a G nucleotide but not bind to a variant with an A, C, or T nucleotide. Similarly, PCR primers could be designed to bind to the SNP having a C or a T nucleotide, but not both, which then does not bind to a variant with a G. A, or T nucleotide or G, A, or C nucleotide respectively. In particular aspects, the PCR primer is at least or no more than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more nucleotides in length with 100% homology to the template sequence, with the potential exception of non-homology the SNP location. After several rounds of amplifications, if the PCR primers generate the expected band size, the SNP can be determined to have the A nucleotide and not the G nucleotide.

### B. Copy Number Variation Detection

Particular aspects of the disclosure concern methods of detecting a copy number variation (CNV), also “copy number alteration” (CNA), of a particular allele. A CNV may be genomic gain. A CNV may be genomic loss. One can utilize any known method for detecting CNVs to detect the CNVs. Such methods include fluorescent in situ hybridization, comparative genomic hybridization, arrays, polymerase chain reaction, sequencing, or a combination thereof, for example. In some aspects, the CNV is detected using an array. Array platforms such as those from Agilent, Illumina, or Affymetrix may be used, or custom arrays could be designed. One example of how an array may be used includes methods that comprise one or more of the steps of isolating nucleic acid material in a suitable manner from an individual suspected of having the CNV and, at least in some cases from an individual or reference genome that does not have the CNV; processing the nucleic acid material by fragmentation, labelling the nucleic acid with, for example, fluorescent labels, and purifying the fragmented and labeled nucleic acid material; hybridizing the nucleic acid material to the array for a sufficient time, such as for at least 24 hours; washing the array after hybridization; scanning the array using an array scanner; and analyzing the array using suitable software. The software may be used to compare the nucleic acid material from the individual suspected of having the CNV to the nucleic acid material of an individual who is known not to have the CNV or a reference genome.

In some aspects, detection of a CNV is achieved by polymerase chain reaction (PCR). PCR primers can be employed to amplify nucleic acid at or near the CNV wherein an individual with a CNV will result in measurable higher levels of PCR product when compared to a PCR product from a reference genome. The detection of PCR product amounts could be measured by quantitative PCR (qPCR) or could be measured by gel electrophoresis, as examples. Quantification using gel electrophoresis comprises subjecting the resulting PCR product, along with nucleic acid standards of known size, to an electrical current on an agarose gel and measuring the size and intensity of the resulting band. The size of the resulting band can be compared to the known standards to determine the size of the resulting band. In some aspects, the amplification of the CNV will result in a band that has a larger size than a band that is amplified, using the same primers as were used to detect the CNV, from a reference genome or an individual that does not have the CNV being detected. The resulting band from the CNV amplification may be nearly double, double, or more than double the resulting band from the reference genome or the resulting band from an individual that does not have the CNV being detected. In some aspects, the CNV can be detected using nucleic acid sequencing. Sequencing techniques that could be used include, but are not limited to, whole genome sequencing, whole exome sequencing, and/or targeted sequencing.

### C. DNA Sequencing

In some aspects, DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some aspects, a unique genetic readout for each sample may be generated by genotyping one or more highly polymorphic SNPs. In some aspects, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage. In certain aspects, mutations, SNPS, INDELS, copy number alterations (somatic and/or germline), or other genetic differences may be identified from the sequencing using at least one bioinformatics tool, including VarScan2, any R package (including CopywriteR) and/or Annovar.

### D. RNA Sequencing

In some aspects, RNA may be analyzed by sequencing. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some aspects, sequencing may be performed to generate approximately 10 M, 15 M, 20 M, 25 M, 30 M, 35 M, 40 M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer. In some aspects, raw sequencing data may be converted to estimated read counts (RSEM), fragments per kilobase of transcript per million mapped reads (FPKM), and/or reads per kilobase of transcript per million mapped reads (RPKM). In some aspects, one or more bioinformatics tools may be used to infer stroma content, immune infiltration, and/or tumor immune cell profiles, such as by using upper quartile normalized RSEM data.

### E. Proteomics

In some aspects, protein may be analyzed by mass spectrometry. The protein may be prepared for mass spectrometry using any method known in the art. Protein, including any isolated protein encompassed herein, may be treated with DTT followed by iodoacetamide. The protein may be incubated with at least one peptidase, including an endopeptidase, proteinase, protease, or any enzyme that cleaves proteins. In some aspects, protein is incubated with the endopeptidase, LysC and/or trypsin. The protein may be incubated with one or more protein cleaving enzymes at any ratio, including a ratio of μg of enzyme to μg protein at approximately 1:1000, 1:100, 1:90, 1:80, 1:70, 1:60, 1:50, 1:40, 1:30, 1:20, 1:10, 1:1, or any range between. In some aspects, the cleaved proteins may be purified, such as by column purification. In certain aspects, purified peptides may be snap-frozen and/or dried, such as dried under vacuum. In some aspects, the purified peptides may be fractionated, such as by reverse phase chromatography or basic reverse phase chromatography. Fractions may be combined for practice of the methods of the disclosure. In some aspects, one or more fractions, including the combined fractions, are subject to phosphopeptide enrichment, including phospho-enrichment by affinity chromatography and/or binding, ion exchange chromatography, chemical derivatization, immunoprecipitation, co-precipitation, or a combination thereof. The entirety or a portion of one or more fractions, including the combined fractions and/or phospho-enriched fractions, may be subject to mass spectrometry. In some aspects, the raw mass spectrometry data may be processed and normalized using at least one relevant bioinformatics tool.

### F. Detection Kits and Systems

One can recognize that based on the methods described herein, detection reagents, kits, and/or systems can be utilized to detect the SNP and/or the CNV related to the genetic signature for diagnosing an individual (the detection either individually or in combination). The reagents can be combined into at least one of the established formats for kits and/or systems as known in the art. As used herein, the terms “kits” and “systems” refer to aspects such as combinations of at least one SNP detection reagent, for example at least one selective oligonucleotide probe, and at least one CNV detection reagent, for example at least one PCR primer. The kits could also contain other reagents, chemicals, buffers, enzymes, packages, containers, electronic hardware components, etc. The kits/systems could also contain packaged sets of PCR primers, oligonucleotides, arrays, beads, or other detection reagents. Any number of probes could be implemented for a detection array. In some aspects, the detection reagents and/or the kits/systems are paired with chemiluminescent or fluorescent detection reagents. Particular aspects of kits/systems include the use of electronic hardware components, such as DNA chips or arrays, or microfluidic systems, for example. In specific aspects, the kit also comprises one or more therapeutic or prophylactic interventions in the event the individual is determined to be in need of.

### EXAMPLES

The following examples are included to demonstrate certain aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### Example 1—ZNRF3 Loss as a Predictor of Metastatic Relapse

In studies described herein, the prevalence of 113 mutation types was quantified in 72 established prostate cancer driver genes or recurrently mutated genomic regions^{7,18} in 1,844 patients with either localized prostate cancer or mCRPC. Differential prevalence of seventy-three established driver mutations was identified, including mutations in the androgen receptor and its enhancer region. Amongst these differentially prevalent driver mutations, twenty-four were present in at least 5% of localized cancers and four were significantly associated with metastatic relapse of localized disease, including genomic gains in MYC and CCND1. In addition, genomic loss of the WNT pathway inhibitor ZNRF3 was associated with WNT pathway activity and predicted biochemical and metastatic relapse and overall survival in localized prostate cancer. These results demonstrated a method of identifying candidate prognostic genomic biomarkers by comparing primary and metastatic disease and establish ZNRF3 loss as a novel predictor of aggressive localized prostate cancer.

### Molecular Drivers of Localized and Metastatic Prostate Cancer

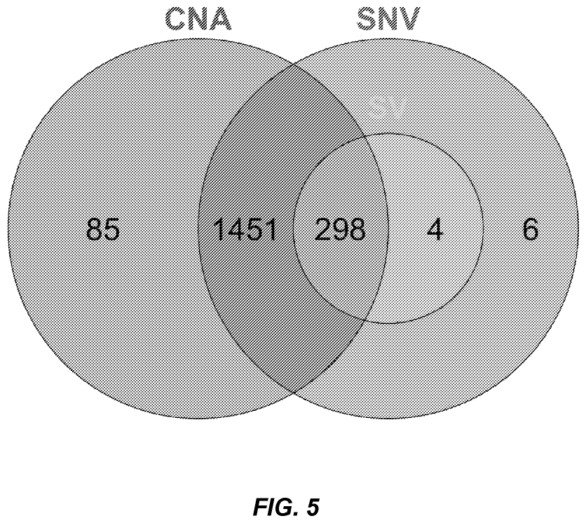

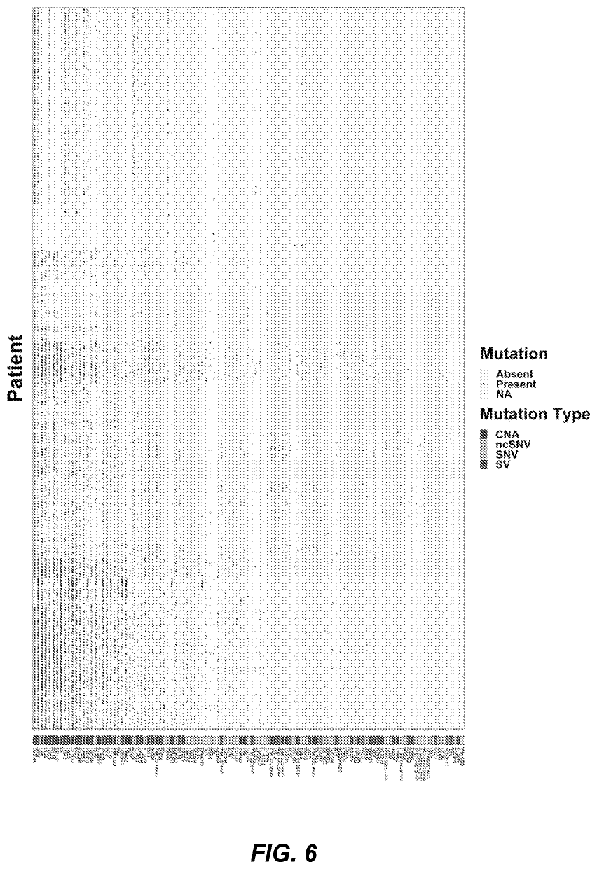

The inventors collected somatic SNV, CNA, and SV calls from eleven DNA sequencing studies of prostate cancer comprising 1,844 patients (1,289 localized, 555 metastatic; FIGS. 5 and 6 and Table 2)^{7,11-15,18,23,26-28}. The inventors curated a list of 113 mutation types—31 CNAs, 43 coding SNVs, 6 non-coding SNVs and 33 SVs—from those identified as either putative drivers or recurrently mutated in two large whole-genome sequencing studies of prostate cancer^{7,18}. These encompassed 72 individual genes or genomic loci (Table 2).

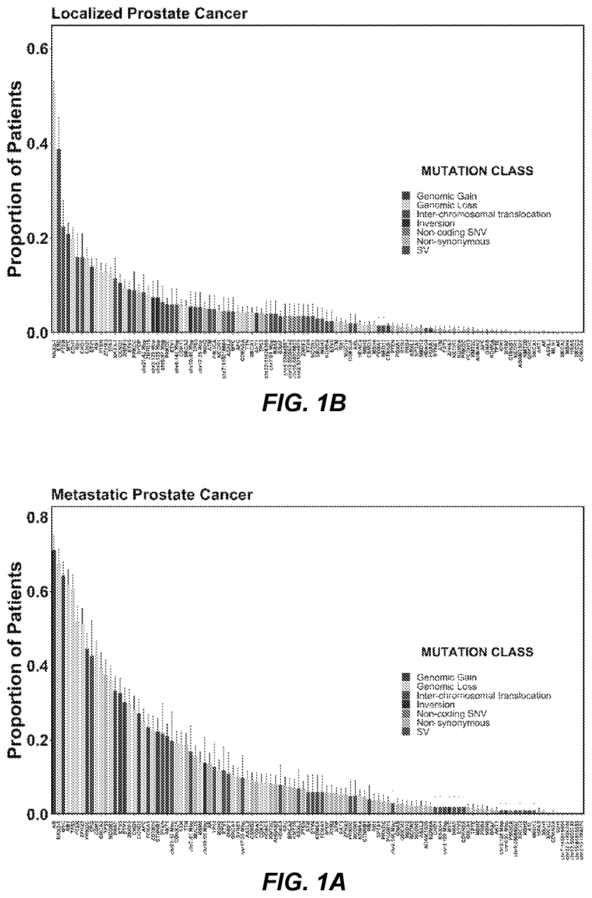

The most common mutation in localized prostate cancers was loss of NKX3-1, in 644/1279 cases (50.4%; FIG. 1A and Table 3). Other mutations present in at least 20% of localized cancers were ERG SVs (78/201 cases; 38.8%), PTEN SVs (45/201 cases; 22.4%), MYC gains (267/1279 cases; 20.9%), and CDH1 losses (256/1279 cases; 20.0%). The most common mutation in mCRPC was gains of the androgen receptor (AR) gene, which occurred in 395/555 cases (71.2%; FIG. 1B and Table 3). Consistent with the higher rate of mutation reported in mCRPC relative to localized disease¹⁶, 25 genes were mutated in at least 20% of mCRPC cases, compared with only 4 in localized prostate cancer (FIG. 1B and Table 3).

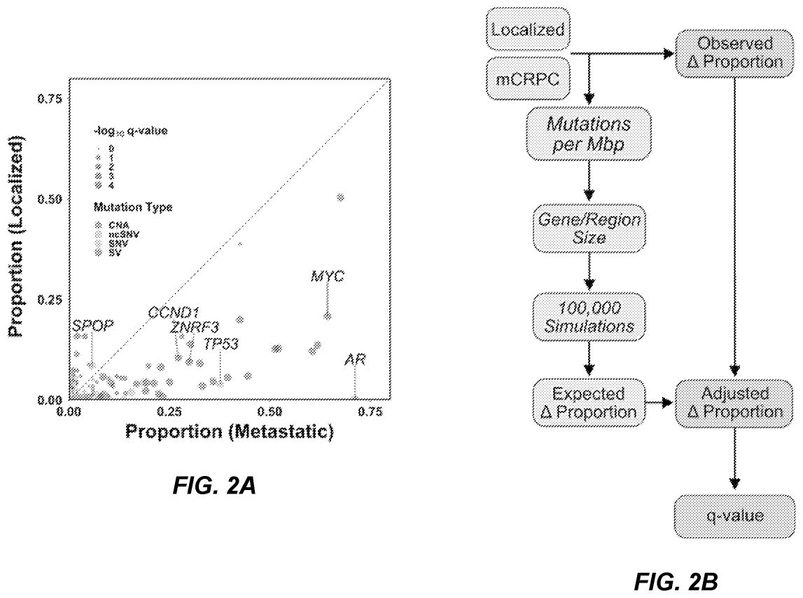

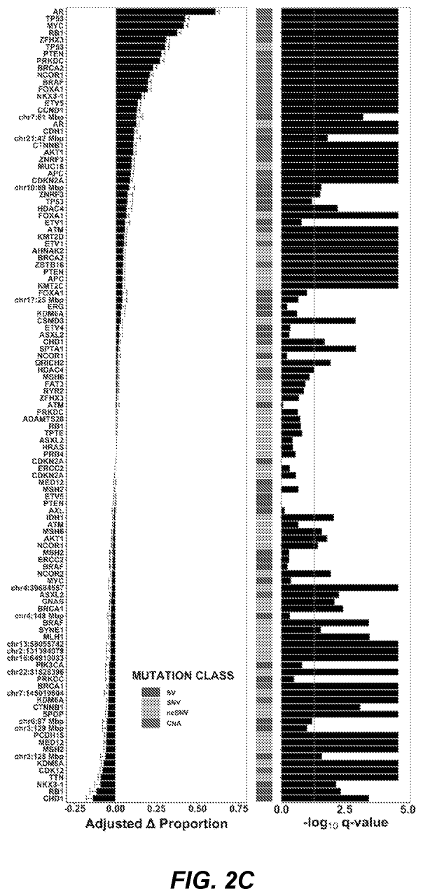

The proportion of localized prostate cancer and mCRPC cases harboring each driver mutation is shown in FIG. 2A. To assess which mutations are more prevalent in each disease state, the inventors first evaluated the difference in these proportions (‘observed Δ proportion’). Because of the differences in mutational burden between localized disease and mCRPC^{18,28}, the inventors also derived an expected Δ proportion based on 100,000 samples of the binomial distribution, per driver gene mutation, per sample, weighted by mutational burden and gene size, in each tumor sample (FIG. 2B). Finally, the inventors computed the difference between observed Δ proportion and expected Δ proportion, yielding an adjusted Δ proportion which indicates whether a driver mutation is prevalent in mCRPC more than expected (adjusted Δ proportion >0) or less than expected (adjusted Δ proportion <0), based on background global mutation burden.

Overall, 37.2% of driver gene mutations (42/113) were significantly more prevalent than expected in mCRPC (q<0.05, adjusted Fisher's Exact test; FIG. 2C and Table 3) 27,42. By comparison, 27.4% (31/113) were significantly less prevalent. Driver mutations more prevalent in mCRPC may either provide a selective advantage to metastasis when localized or may result from adaptation to metastatic niches and response to therapy. The largest adjusted Δ proportion was for genomic gain of the AR locus (gene body or enhancer), which was present in 395/555 (71.2%, 95% CI: 67.4-74.9) mCRPCs but only 2/1,279 (0.16%; 95% CI: 0-0.37) localized cancers (adjusted Δ proportion=0.609, 95% CI: 0.587-0.632, q=2.35×10⁻⁵, adjusted Fisher's Exact test; FIG. 2C). SNVs in AR were less common overall and were not observed in localized disease (76/555 vs. 0/1,204; adjusted Δ proportion=0.128, 95% CI: 0.113-0.144, q=2.35×10⁻⁵, adjusted Fisher's Exact test). CNAs in BRCA2 were frequently observed and significantly more prevalent in mCRPC than expected (219/555 vs. 72/1,279; adjusted Δ proportion=0.232, 95% CI: 0.212-0.251, q=2.35×10⁻⁵, adjusted Fisher's Exact test), consistent with reports showing that germline and somatic aberrations in BRCA2 are more prevalent in mCRPC than localized disease^{43,44}. CNAs and SNVs in TP53 were both significantly more prevalent in mCRPC (SNV: 205/555 vs. 49/1,204; adjusted Δ proportion=0.305, 95% CI: 0.284-0.332, q=2.35×10⁻⁵; CNA: 336/555 vs. 156/1,279; adjusted Δ proportion=0.426, 95% CI: 0.403-0.448, q=2.35×10⁻⁵, adjusted Fisher's Exact test). Similarly, SVs affecting TP53 were more prevalent in metastatic disease (adjusted Δ proportionsv=0.074, 95% CI: 0.044-0.104, q=0.060, adjusted Fisher's Exact test). While this did not reach the pre-set statistical significance threshold, it is important to note the lower power to detect differences in SV prevalence, relative to other mutational classes. Consistent with previous reports^{16,45}. SPOP SNVs were less prevalent in mCRPC¹⁶ (32/555 vs. 103/1,204; adjusted Δ proportion=−0.051, 95% CI: −0.061-0.041; q=2.35×10⁻⁵, adjusted Fisher's Exact test).

### Candidate Genomic Biomarkers of Aggressive Localized Prostate Cancer

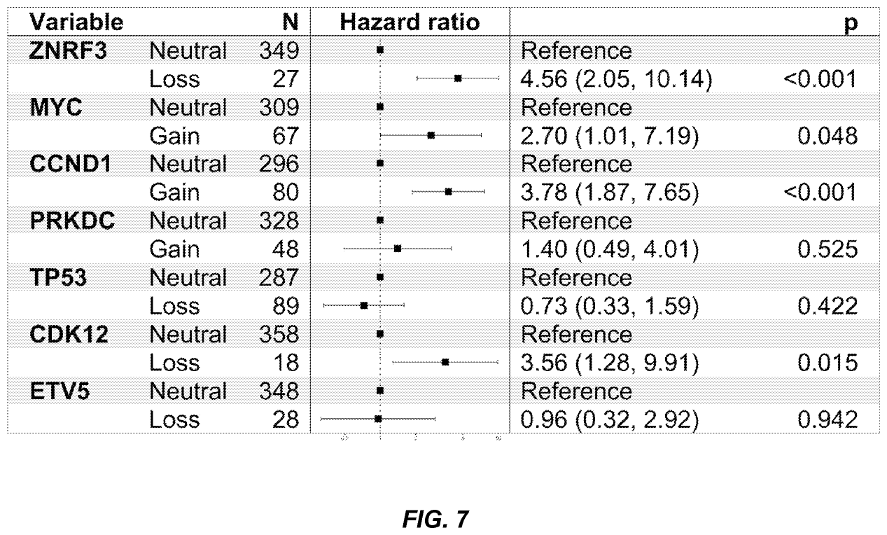

Relapse of localized prostate cancer following curative-intent therapy is due, at least in part, to the presence of occult metastatic disease at initial presentation. The inventors hypothesized that mutations prevalent in mCRPC than localized disease might be prognostic for relapse of localized prostate cancer. From the list of 73 mutations with significantly different prevalence in mCRPC vs. localized disease (42 with increased prevalence, 31 with lower prevalence), the inventors identified 24 that occurred in at least 5% of all localized cancers evaluated (Table 4). These 24 driver mutations comprised 16 CNAs, 6 SVs, and 2 SNVs. The inventors then used univariable Cox proportional hazards modeling to assess whether any of these 24 mutations are associated with metastatic relapse of localized disease. Using patient outcome data from the CPCG study (n=376), 4 of the 24 driver mutations—all CNAs—that were more prevalent in mCRPC were also associated with significantly higher risk of metastatic relapse (q<0.01; Table 5): MYC, CCND1, and PRKDC gain & ZNRF3 loss, while three other mutations—losses of CDK12, ETV5, and TP53—were associated with metastatic relapse but did not reach the pre-determined level of statistical significance (q<0.05). PTEN and RB1 loss were not prognostic of metastatic relapse. In a multivariable Cox proportional hazards model including all seven driver CNAs with q≤0.25, ZNRF3 loss, MYC gain, CCND1 gain, and CDK12 loss remained significantly associated with risk of metastatic relapse (N_{METS}=36/376; FIGS. 7A-7B).

To confirm these associations with adverse outcome, the inventors employed two independent validation cohorts: TCGA PRAD²³ and MSKCC²⁹ localized prostate cancer cohorts. In TCGA, MYC and CCND1 gains as well as ZNRF3 losses were prognostic for progression-free survival (Table 6A); on multivariable analysis, only ZNRF3 remained prognostic (Table 6A). Similarly, these CNAs were also prognostic for poor outcomes in the MSKCC cohort (Table 6B and 6C). Taken together, these data support the utility of this approach to prognostic biomarker discovery.

ZNRF3 Genomic Loss is Associated with Gene Expression and Clinical Features

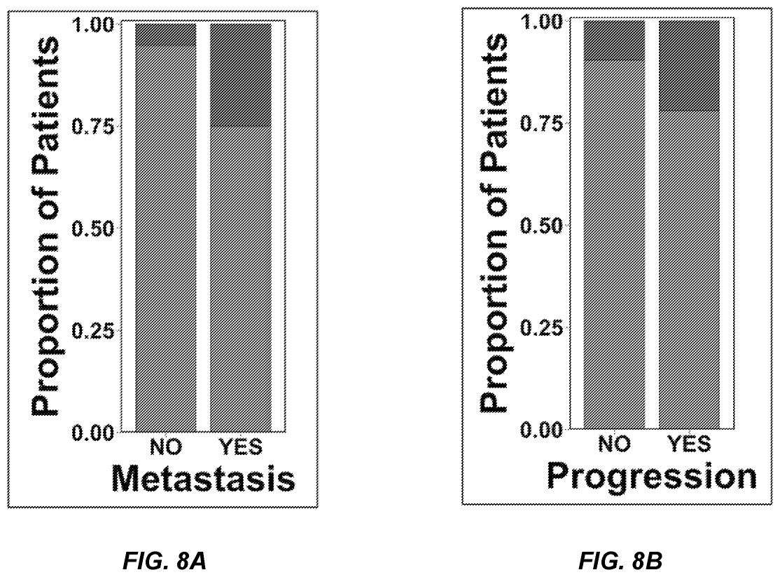

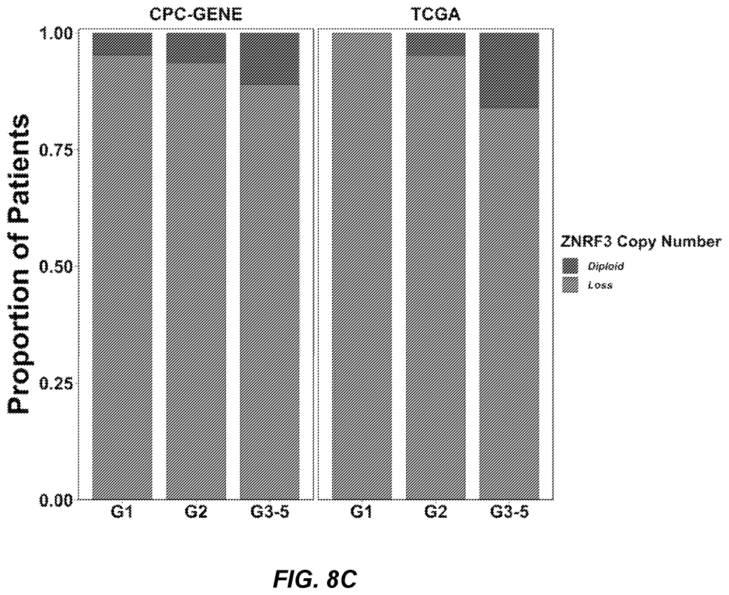

Because it represents a potentially novel biomarker of adverse outcome, the inventors focused on the functional and clinical implication of ZNRF3 loss. ZNRF3 loss was identified in 166/555 metastases (29.9%; 95% CI: 26.1-33.7) and 122/1,279 localized cancers (9.54%; 95% CI: 7.93-11.1; adjusted Δ proportion=0.10, 95% CI: 0.086-0.113, q=2.35×10⁻⁵, adjusted Fisher's Exact test). While ZNRF3 loss was present in ˜10% of localized cancers overall, this CNA was enriched in aggressive disease. In CPC-GENE, 25% of patients (9/36) who relapsed metastatically harboured ZNRF3 loss while only 5.2% of patients (18/341) who did not harbour it (OR=5.98, 95% CI: 2.45-14.6; p=3.29×10⁻⁴, Fisher's Exact test; FIG. 8A). Similarly, in the TCGA cohort, ZNRF3 loss was identified in 11.9% (58/489) of patients; of the 91/489 patients with disease progression, 20/91 (22.0%) harboured ZNRF3 loss while only 9.55% of patients who did not progress harboured this CNA (OR=2.67, 95% CI: 1.47-4.85; p=1.64×10⁻³, Fisher's Exact test; FIG. 8B). ZNRF3 loss was also associated with higher grade tumours in both CPCG and TCGA (FIG. 8C). Thus, ZNRF3 loss appears to identify a novel subtype of localized prostate cancer associated with aggressive clinical outcomes.

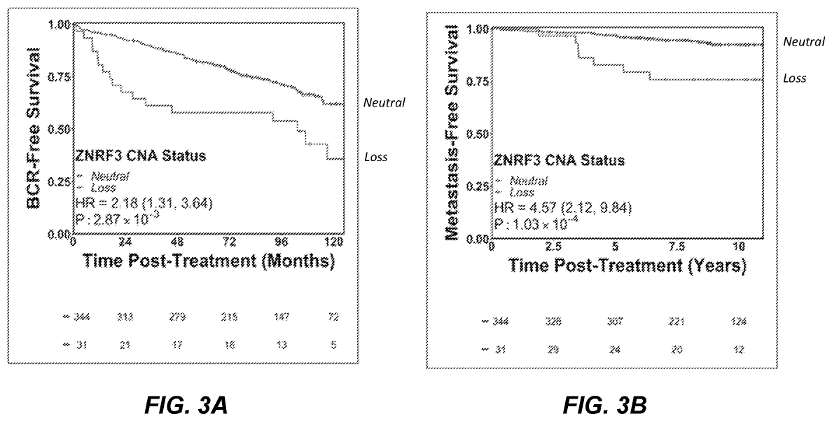

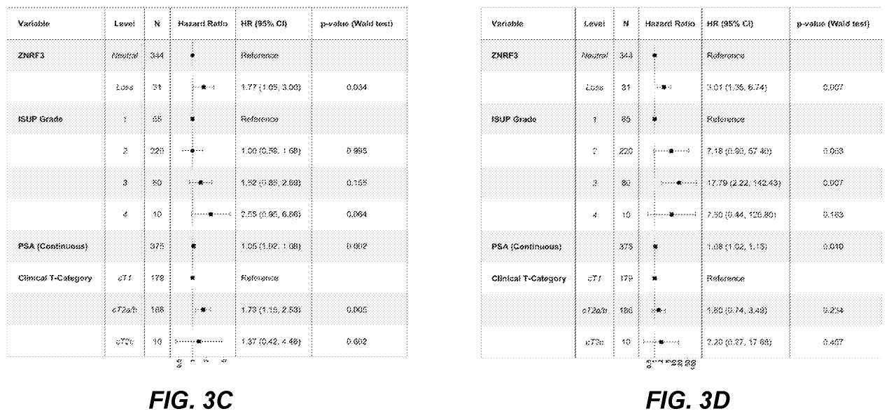

ZNRF3 loss was prognostic for BCR and metastatic relapse on univariable Cox proportional hazards analysis in CPCG (HR_{BCR}=2.18, 95% CI: 1.31-3.64, p=2.87×10⁻³, Wald test; HR_{METS}=4.57, 95% CI: 2.12-9.84, p=1.03×10⁻⁴; Wald test; FIGS. 3A and 3B). These effects remained significant after controlling for standard clinical prognostic variables (i.e. pre-treatment PSA, diagnostic ISUP grade group, and clinical T category) in multivariable Cox proportional hazards modeling (HR_{BCR}=1.77, 95% CI: 1.05-3.00, p=0.034, Wald test; N_{BCR}=126/376; HR_{METS}=3.01, 95% CI: 1.35-6.74, p=7.32×10⁻³, Wald test; N_{METS}=36/376; FIGS. 3C and 3D; univariable Cox models for all clinical prognostic factors are shown in Table 7).

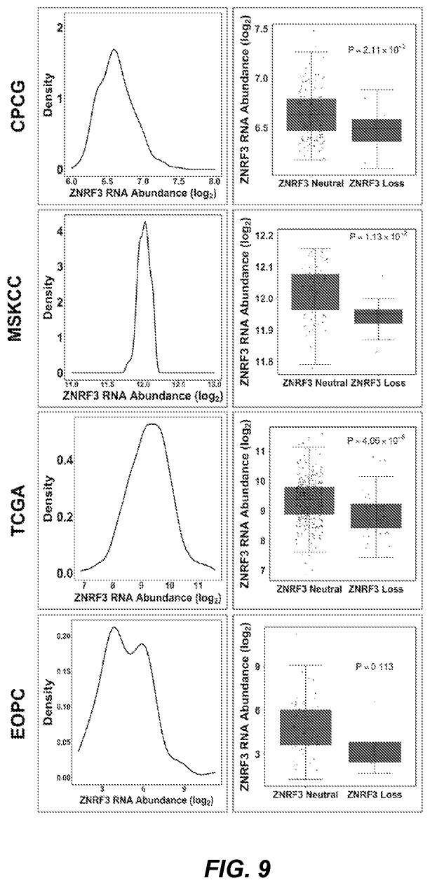

To validate the transcriptomic impact of ZNRF3 genomic loss, the inventors assessed ZNRF3 RNA abundance in 208 CPCG tumors with matched RNA abundance and CNA data. ZNRF3 RNA abundance spans a limited range across tumour specimens (mean log 2 RNA abundance=6.63, range: 6.09-7.47; FIG. 9). Tumors harboring a genomic loss in ZNRF3 had significantly lower ZNRF3 RNA abundance than ZNRF3 neutral tumours (mean log 2 RNA abundance difference=0.163, 95% CI: 0.132-0.195, p=0.021, Mann-Whitney U-test; FIG. 9). This association validated in TCGA (mean log 2 RNA abundance difference=0.450, 95% CI: 0.303-0.596, p=4.06×10⁻⁶, Mann-Whitney U-test; FIG. 9) and Taylor/MSKCC cohorts (mean log 2 RNA abundance difference=0.075, 95% CI: 0.070-0.079, p=1.13×10⁻², Mann-Whitney U-test; FIG. 9). Only four patients in the Gerhauser cohort with ZNRF3 loss had available RNA abundance data, but there was a trend toward decreased ZNRF3 RNA abundance relative to ZNRF3 neutral patients (mean log 2 RNA abundance difference=1.37, 95% CI: −1.42-4.16, p=0.11, Mann-Whitney U test; FIG. 9).

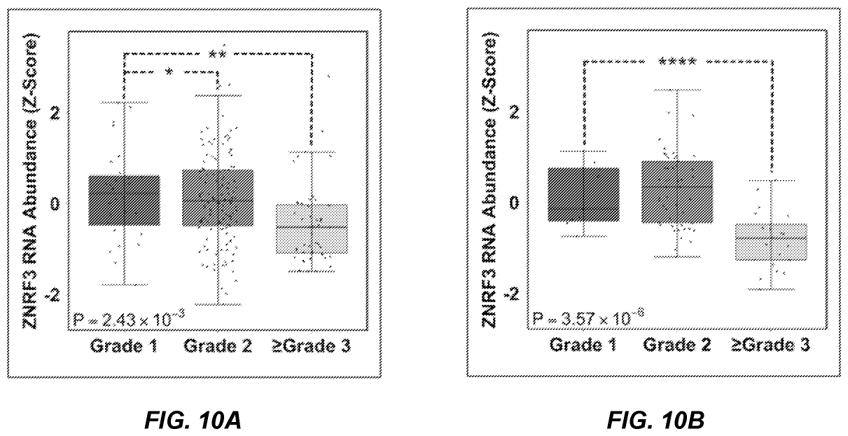

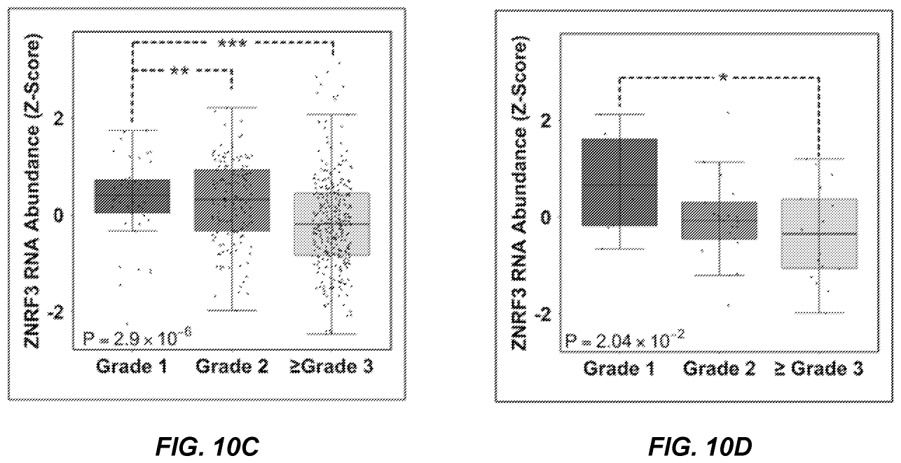

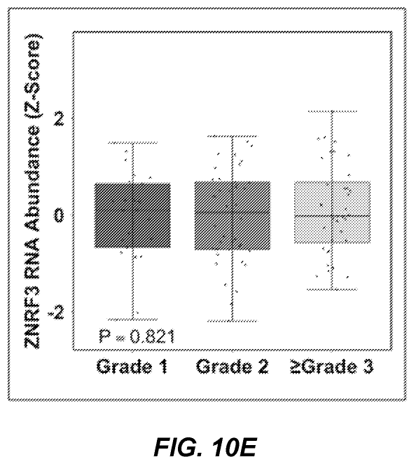

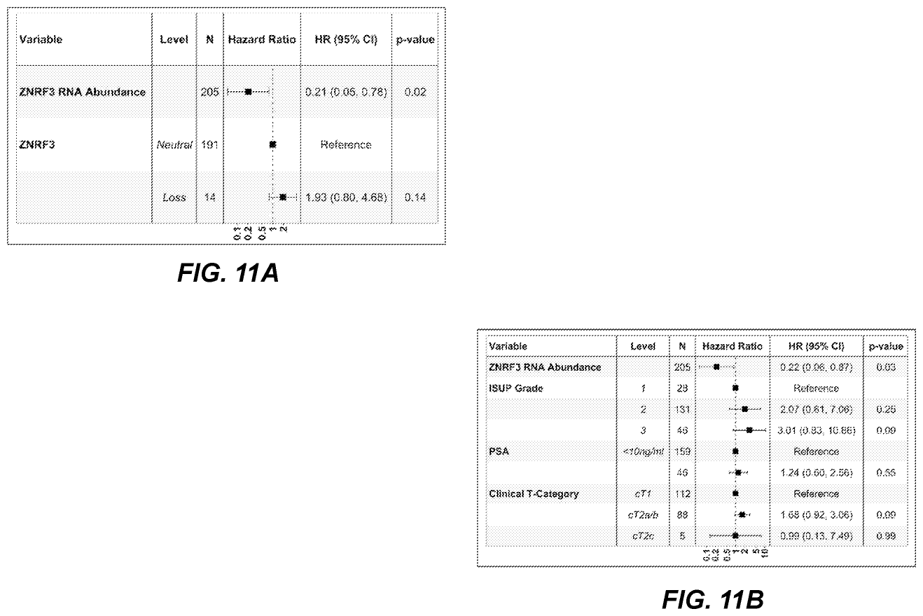

ZNRF3 RNA abundance was inversely associated with ISUP grade in four of five independent cohorts of localized prostate cancer cohorts (FIGS. 10A-10E). Likewise, in CPCG, ZNRF3 RNA abundance was inversely associated with risk of BCR on multivariable analysis controlling for clinical ISUP grade, pre-treatment PSA, and clinical T-category (N_{BCR}=46/205; HR_{BCR}=0.22, 95% CI: 0.06-0.86, p=0.030, Wald test; FIG. 11A). ZNRF3 RNA abundance was not significantly associated with metastatic relapse on multivariable analysis, potentially due to a lack of statistical power resulting from the low event rate in this intermediate risk cohort (N_{METS}=16/205 patients; HR_{METS}=0.20, p=0.18, Wald test). ZNRF3 RNA abundance remained inversely associated with BCR risk after controlling for ZNRF3 loss in multivariable Cox proportional hazards analysis (HR_{BCR}=0.21, 95% CI: 0.05-0.78, p=0.02, Wald test; N_{BCR}=46/205; FIG. 11B).

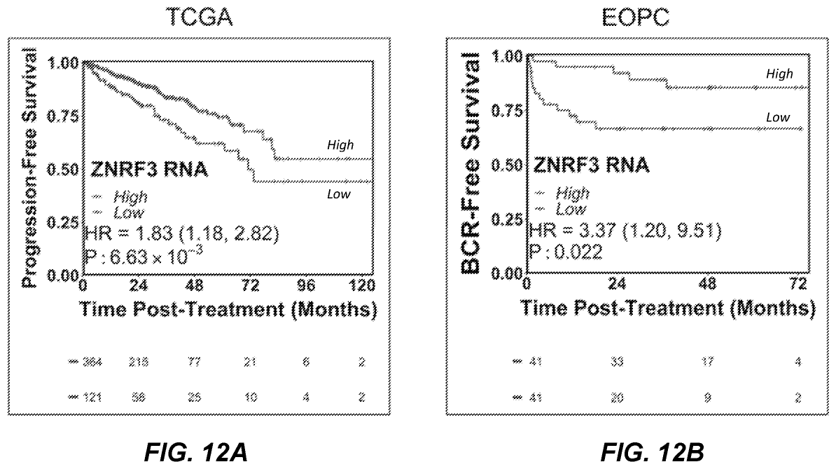

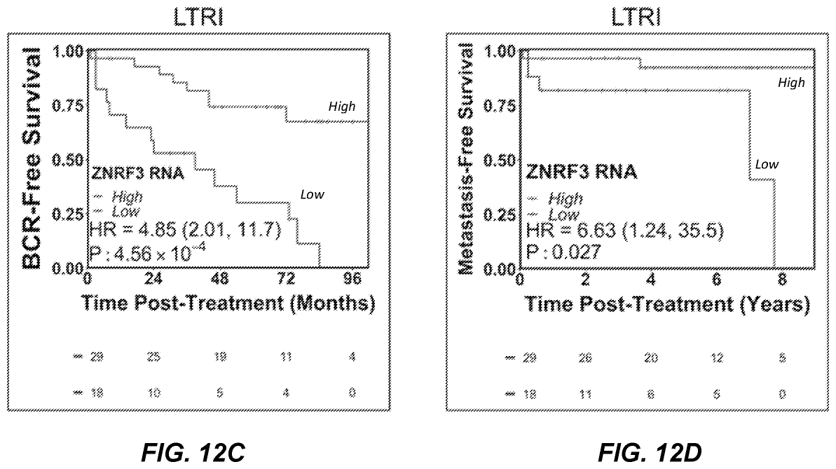

The inventors validated this association between ZNRF3 RNA and risk of adverse clinical outcome in three independent cohorts: TCGA, EOPC, and a novel high risk/high-volume intermediate risk cohort (LTRI), in which low ZNRF3 RNA abundance was associated with a significantly higher risk of PFS, BCR, and metastatic relapse (FIGS. 12A-12D). In the LTRI cohort, 18/18 patients with low ZNRF3 RNA abundance experienced metastatic relapse within 7 years following surgery, compared with only 2/29 patients with high ZNRF3 RNA abundance (p=4.16×10⁻¹¹, Fisher's Exact test; FIG. 12D). ZNRF3 RNA was not related to risk of disease progression in the Taylor/MSKCC cohort. Taken together, these data demonstrate that genomic loss or low RNA abundance of ZNRF3 preferentially occur in aggressive localized prostate cancer, independent of standard clinical prognostic factors.

### ZNRF3 Loss Predicts Poor Outcome Localized Prostate Cancer

The inventors recently developed a six-feature clinico-genomic signature that predicts biochemical relapse in men with localized prostate cancer⁷. To assess whether ZNRF3 genomic loss adds independent prognostic value to these features, the inventors stratified the CPCG cohort based on both signature features and ZNRF3 loss. Overall, 298/379 (78.6%) CPCG cases had informative data for the six features in the signature (MYC gain, ATM SNVs, TCERGL1 hypomethylation, ACTL6B hypermethylation, chr7:61 Mbp inter-chromosomal translocations, and clinical T category). On multivariable analysis, both the Fraser signature and ZNRF3 remained prognostic of BCR and metastasis, after controlling for ISUP grade group, PSA, and clinical T category (HR_{BCR}=1.77, 95% CI: 1.06-2.98, p=0.030, Wald test; N_{BCR}=126/375; HR_{METS}=2.86, 95% CI: 1.29-6.34, p=0.019, Wald test; N_{METS}=36/375; Table 8).

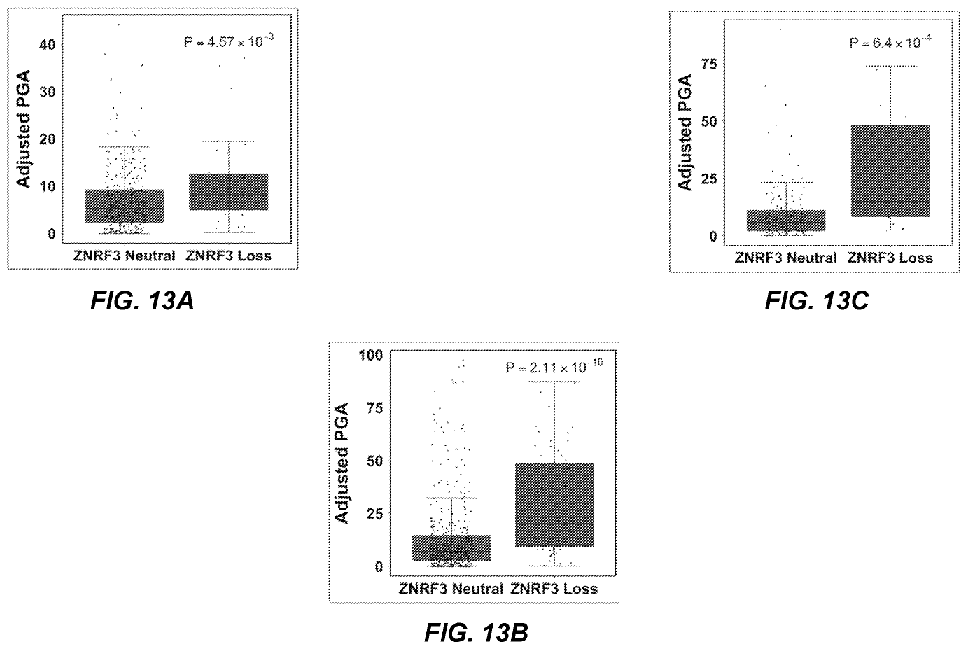

In the CPCG cohort, ZNRF3 genomic loss was associated with significantly higher percentage of the genome altered by a CNA (Mean Adjusted PGA: 6.96%, 95% CI: 6.27-7.66 vs. 11.1%, 95% CI: 7.66-14.5, p=4.57×10⁻³, Mann-Whitney U test; FIG. 13A), and this validated in the TCGA (Mean Adjusted PGA: 13.2%, 95% CI: 11.5-15.0 vs. 30.0%, 95% CI: 24.0-36.1, p=2.11×10⁻¹⁰. Mann-Whitney U test; FIG. 13B) and Taylor/MSKCC cohorts (Mean Adjusted PGA: 27.8%, 95% CI: 14.2-41.4 vs. 9.45%, 95% CI: 7.51-11.4, p=6.40×10⁻⁴, Mann-Whitney U test; FIG. 13C). Despite this, ZNRF3 genomic loss remained prognostic of metastatic relapse in the CPCG cohort after correcting for adjusted PGA, PSA, ISUP grade, clinical T category, and intraductal carcinoma of the prostate/cribriform architecture (IDC-P/CA)—an established negative prognostic factor^{51,52}—in multivariable Cox proportional hazards models (HR_{METS}=3.53, 95% CI: 1.47-8.47, p=4.72×10⁻³, Wald test; N_{METS}=30/326; Table 1). In contrast, ZNRF3 loss was not prognostic of BCR after correcting for these clinical and pathologic prognostic features (HR_{BCR}=1.49, 95% CI: 0.83-2.67, p=0.180, Wald test; N_{BCR}=106/326). These results support the hypothesis that ZNRF3 loss is an independent predictor of metastatic relapse in localized prostate cancer.

ZNRF3 loss occurred in the context of a relatively large region of genomic loss on chromosome 22q12.1 (median deletion: 6.0 Mbp, range: 1.01-35.1 Mbp) and the smallest number of genes co-deleted with ZNRF3 was 10 (median co-deleted genes=90), covering 1.31 Mbp. All 417 genes on chromosome 22 were co-deleted with ZNRF3 in at least one patient. Of these, 29 (5.3%) were significantly associated with metastatic relapse in CPCG (q<0.01, Wald test; Table 9). Of these 29 genes co-deleted with ZNRF3 and associated with metastasis, 9/29 also showed significant reduction in RNA abundance in cases with genomic loss in this region (q<0.05, Mann-Whitney U test), and thus represent likely candidate drivers of the aggressive phenotype associated with this deletion. To further refine this gene list and help to identify the driver of aggression in this region, the inventors next determined the association between RNA abundance of these 9 genes and adverse clinical outcomes in four independent cohorts (CPCG, TCGA, EOPC, and LTRI) using univariable Cox proportional hazards models (Table 10). The only gene with RNA abundance significantly associated with adverse outcome across all four cohorts (i.e. q<0.05) was ZNRF3. These data are thus consistent with the hypothesis that ZNRF3 loss drives clinical aggression of localized prostate cancer.

### Molecular Hallmarks of ZNRF3 Genomic Loss

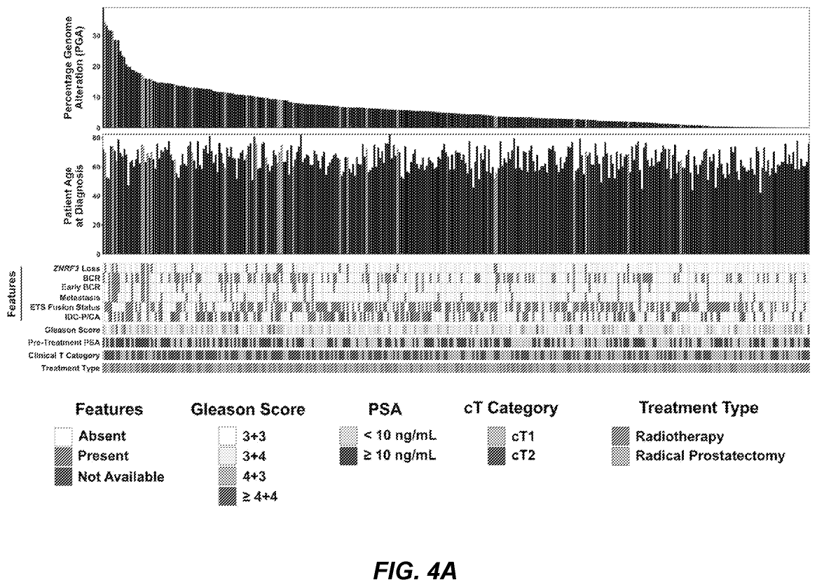

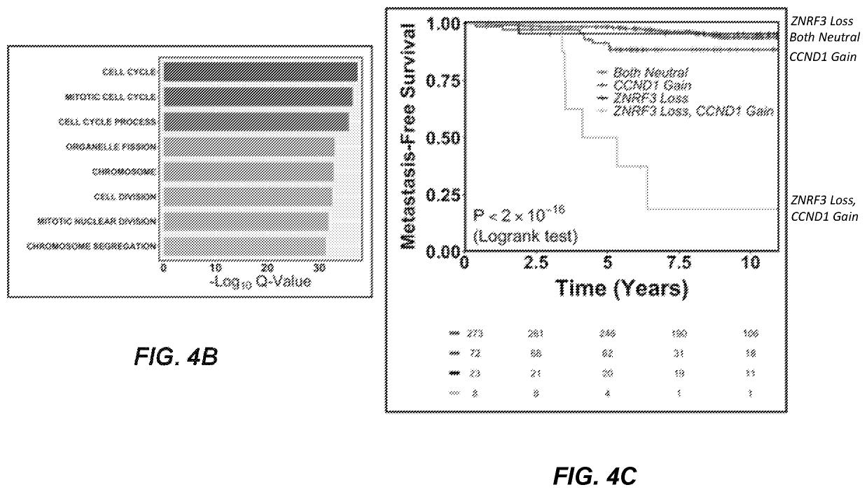

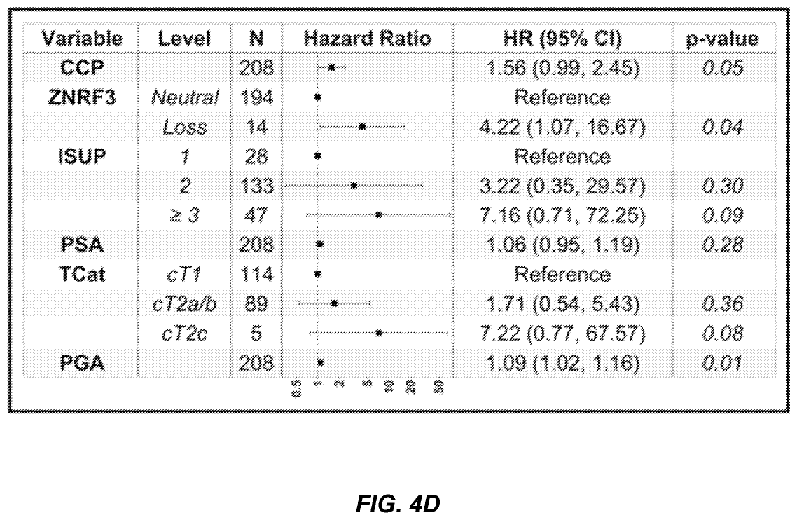

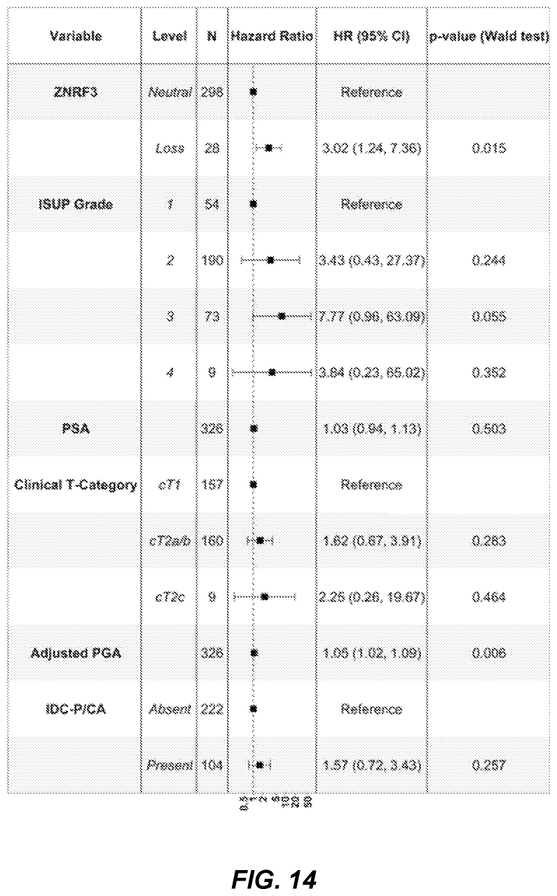

The inventors next assessed associations between ZNRF3 loss and clinical, pathological, and genomic features in the CPCG cohort (FIG. 4A). ZNRF3 genomic loss was not associated with age at diagnosis (q=0.491, Mann-Whitney U test). Moreover, ZNRF3 genomic loss was not associated with the presence of IDC/CA, either across the full cohort (q=0.491, Fisher's Exact test) or when patients were stratified by ISUP grade group (to account for the increased prevalence of IDC/CA in higher grade tumors^{53,54}; ISUP grade group 1: q=1; ISUP grade group 2: q=0.4317; ISUP grade groups 3 and 4: q=1; Fisher's Exact tests). IDC/CA was prognostic of metastatic relapse on univariable analysis (HR_{METS}=2.14, 95% CI: 1.12-4.52, p=0.046, Wald test), as previously reported^{55,56}. However, on multivariable analysis including ZNRF3 loss, ISUP grade, PSA, clinical T-category, and adjusted PGA (N_{METS}=30/326; FIG. 14), IDC/CA was not prognostic. ZNRF3 loss was not significantly associated with ETS fusion status (Table S11), nor with chromothripsis (p=0.208, Fisher's Exact test), kataegis (q=0.298, Fisher's Exact test), global SNV burden (p=0.376, Mann-Whitney U test), or tumor hypoxia, measured either using a consensus RNA abundance surrogate signature¹⁹ (p=0.518, Mann-Whitney U Test) or by direct intratumoral oxygen measurements^{30,57} (p=0.550, Mann-Whitney U Test). The inventors observed a significant enrichment of CNAs in APC (loss) or CTNNB1 (gain) in tumours harbouring ZNRF3 loss (APC Loss+ZNRF3 Loss: OR=2.08, q=1.06×10⁻⁴, Fisher's Exact test; CTNNB1 Gain+ZNRF3 Loss: OR=1.92, q=2.50×10⁻³, Fisher's Exact test; Either CNA+ZNRF3 Loss: OR=2.24, q=9.81×10⁻⁷, Fisher's Exact test). SNVs in either APC or CTNNB1 were not associated with ZNRF3 loss (APC: p=0.843; CTNNB1: q=1, Fisher's Exact tests). On multivariable analysis, ZNRF3 and APC loss were independently prognostic of PFS in TCGA (N_{PES}=91) and only ZNRF3 loss was independently prognostic of metastatic relapse in CPCG (N_{METS}=36; Table 11).

To further understand the functional correlates of ZNRF3 in prostate cancers, the inventors determined global RNA abundance patterns related to ZNRF3. To maximize the likelihood of identifying functionally important correlations, the inventors looked for associations with ZNRF3 loss in both the CPCG and TCGA cohorts. Using the Molecular Signatures Database (MSigDB; available on the world wide web at software.broadinstitute.org/gsea/msigdb/annotate.jsp), the inventors identified 140 genes with a Spearman's ρ≥0.4 in both cohorts. Of these, five were located on chromosome 22, and were therefore excluded from the downstream analyses. The remaining 135 genes were enriched for genes downregulated in metastatic prostate cancer⁵⁸ (13/135; q=2.2×10⁻⁷, Hypergeometric test) and for genes involved in positive regulation of canonical WNT signaling (GO: 0060070; 9/140, q=7.57×10⁻⁴. Hypergeometric test); genes implicated in WNT signaling with significantly different RNA abundance in both CPCG and TCGA cases harbouring ZNRF3 loss are shown in Table S14. These data suggest that ZNRF3 downregulation in localized prostate cancer may activate WNT signaling, an established driver of mCRPC^{16,27}.

ZNRF3 Loss is Associated with Activation of Cell Cycle Progression Pathways.

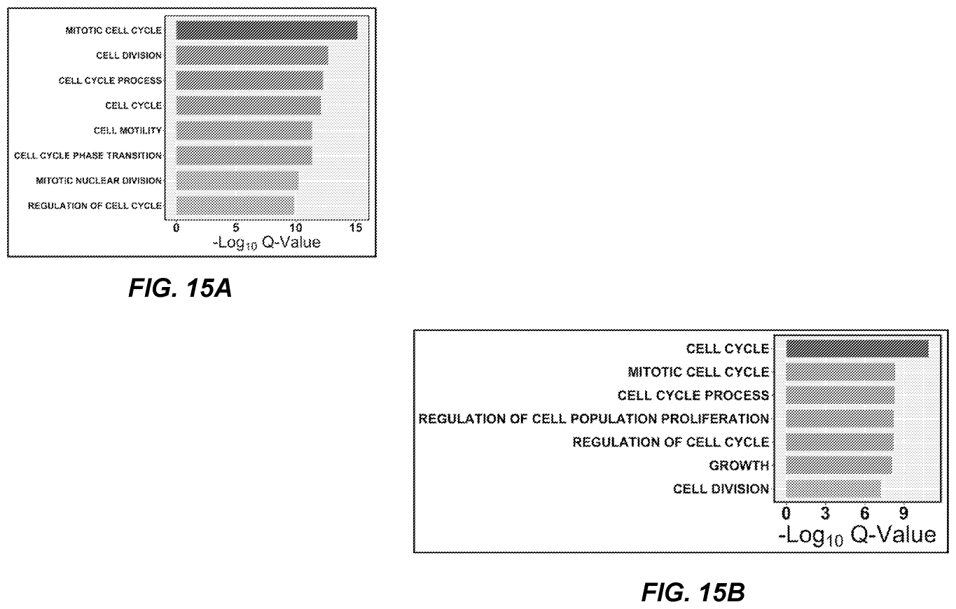

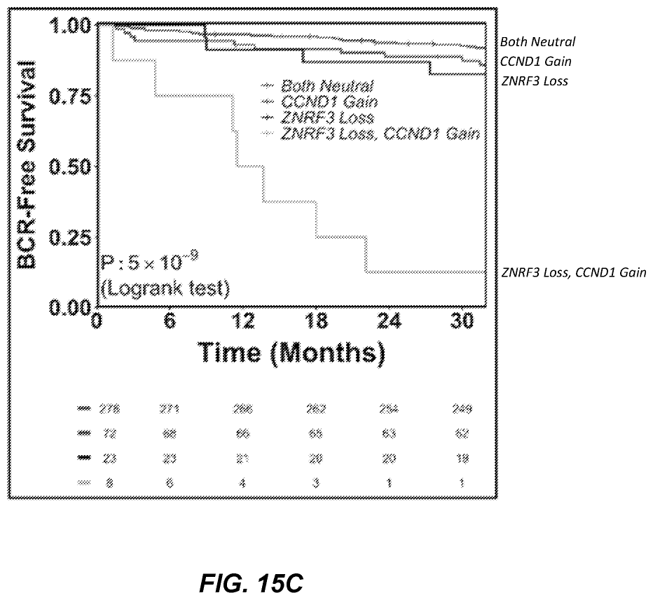

To assess the potential functional role of ZNRF3 loss in localized prostate cancer, the inventors performed Gene Set Enrichment Analysis (GSEA) in tumors with or without ZNRF3 loss. The inventors initially focused on TCGA cases to capitalize on the larger number of samples with RNA abundance data (n=493). GSEA of Gene Ontology (GO) pathways revealed enrichment of cell cycle progression gene sets in tumors harboring ZNRF3 loss (FIG. 4B), which validated in two independent cohorts of both localized and metastatic prostate cancer (FIG. 7A-B). Given the enrichment of cell cycle progression gene sets in tumors harboring ZNRF3 loss, the inventors evaluated clinical outcomes in patients with ZNRF3 genomic loss, with or without amplification of CCND1. The inventors focused on CCND1 because its gain was significantly more prevalent in mCRPC than localized disease and was itself associated with metastatic relapse of localized disease; FIG. 2A and Table 5). While only 8 patients harbored CNAs of both ZNRF3 and CCND1, these patients were at significantly elevated risk of BCR and metastatic relapse (p<2×10⁻¹⁶, log-rank test; FIGS. 4C and 15C). To further confirm the effects of ZNRF3 and cell proliferation, the inventors assessed the interplay between ZNRF3 loss and a clinically-validated RNA-based prognostic biomarker (Prolaris/CCP), which is based on abundance of 31 genes related to cell cycle progression³⁹. CCP score predicted risk of metastasis (HR=1.51, 95% CI: 0.985-2.33, p=0.059, Wald test) in CPCG. However, on multivariable Cox proportional hazards analysis in CPCG, including CCP score, ZNRF3 loss, PGA, and clinical prognostic factors, only ZNRF3 loss and PGA remained prognostic of metastatic relapse (N_{METS}=17/208; FIG. 4D).

Comparison of primary and metastatic tumor genomics provides an attractive strategy for prognostic biomarker discovery. As described herein, such a strategy has been applied to identify ZNRF3 as a predictor of metastatic relapse in localized prostate cancer. Pre-treatment evaluation of ZNRF3 tumour genomic loss and RNA abundance may improve treatment stratification for men with localized prostate cancer.

### Materials and Methods

### Patient Cohorts, Pathology and Tissue Procurement

Patients in the Canadian Prostate Cancer Genome Network (CPCG) cohort (n=385) were consented for whole-genome sequencing and other molecular analyses, with approval from local Research Ethics Boards (UHN #11-0024 and 06-0822; CHUQ 2012-913: H12-03-192). All patients had National Comprehensive Cancer Network (NCCN) intermediate risk prostate cancer, were treated with radical prostatectomy (RP) or external-beam image-guided radiotherapy (IGRT) and were hormone- and chemotherapy-naïve at the time of treatment. Whole-blood or buffy coat specimens were acquired at the time of consenting. For patients undergoing RP, a fresh frozen specimen was obtained from the index lesion within the resected prostate. For patients undergoing IGRT, an ultrasound-guided biopsy to the index lesion was obtained prior to the start of radiotherapy and was flash frozen in optimal cooling temperature (OCT) medium. For all specimens, 20×10 μm sections were acquired, with a hematoxylin and eosin (H&E)-stained 5 μm section on the top and bottom, as well as between the 10th and 11th section, to confirm continuity of histological features. All specimens were independently audited by two urogenital pathologists for Gleason/ISUP grade⁷, tumour cellularity, and presence of intraductal carcinoma of the prostate (IDC-P) and cribriform architecture (CA) histology. Specimens of at least 70% tumour cellularity were used for molecular analyses. Genomic DNA was extracted using phenol: chloroform, as previously reported^{7,10}. Double-stranded DNA quantity was assessed using a Qubit fluorometer and quality was assessed using a Nanodrop spectrophotometer and a BioRad Bioanalyzer, as previously reported⁷. All clinical, pathological, and molecular data for the CPCG cohort have been reported elsewhere^{7,19-22}

Molecular, clinical, and pathologic data for patients in the TCGA PRAD cohort (PanCancer Atlas; n=494)²³ were obtained from cBioPortal^{24,25} (available on the World Wide Web at cbioportal.org) and the NIH Genomic Data Commons Data Portal (available on the World Wide Web at portal.gdc.cancer.gov). Similar data were obtained from the publicly-available Baca¹¹, Berger¹², Weischenfeldt¹³, Barbieri²⁶, Gerhauser¹⁴, Robinson²⁷, and Abida²⁸ cohorts. Where an individual patient sample was included in multiple reports (e.g. samples from the Baca, Berger, Barbieri, Weischenfeldt, and TCGA studies were included in a meta-analysis along with CPCG samples⁷, the Robinson/Abida SU2C mCPRC cohorts^{27,28} contained 125 patients that were analysed in both studies), all cases were audited using original sample and patient identifiers across studies to ensure no duplication of patients. Data from the Quigley cohort¹⁸ was downloaded from the authors' website, as described below. Data for the Taylor/MSKCC validation cohort²⁹ was obtained from cBioPortal.

Patients in the Mt Sinai Hospital cohort (‘Mt Sinai’; n=47) were consented for molecular analysis, with approval from the Research Ethics Board at Mt. Sinai Hospital and the Lunenfeld Research Institute (MSH REB #14-0211-E and University of Toronto REB #35275). All patients underwent RP for localized high-volume intermediate risk or high-risk prostate cancer and total RNA was extracted from fresh-frozen specimens (see below).

The final cohort for molecular discovery consisted of 1,844 unique patient samples from the Abida, Baca, Barbieri, Berger, CPCG, Gerhauser, Quigley, Robinson, TCGA, and Weischenfeldt studies. For clinical outcome analyses (see below), CPCG was used for discovery, with validation in the Gerhauser (EOPC), Mt. Sinai (LTRI), Taylor, and TCGA cohorts.

### Selection of Driver Aberrations

A list of 113 mutations across 72 established driver genes or recurrently altered loci was compiled as the union of drivers reported in the Quigley and Fraser studies^{7,18} (Table 2). Mutations included copy number aberrations (CNAs), coding single nucleotide variants (SNVs; non-synonymous, stop codon gained, stop codon lost, and splice gain/loss), non-coding single nucleotide variants (ncSNVs) and non-CNA structural variants (translocations, fusions, inversions; SVs). While frameshift indels can also result in stop codon losses, the inventors did not include these mutations in the current analysis, which focused only on established driver mutations. Mutations in the androgen receptor enhancer (624 kb upstream of AR¹⁸; CNAs only) were pooled with those in the AR gene body. For ncSNV drivers identified in CPCG⁷, hg19 coordinates were mapped to GRCh38 using the NCBI Remap web interface (available on the World Wide Web at ncbi.nlm.nih.gov/genome/tools/remap) to compare across cohorts. In cases where a gene was subject to multiple mutation types (e.g. TP53 SNVs. CNAs, and SVs), each mutation was analyzed independently. Because not all mutation types were available for all cohorts, the denominator was different for different types:
Coding SNV: 1,204 localized+555 mCRPC=1,759 specimens
Non-Coding SNV: 201 localized+101 mCRPC=302 specimens
CNA: 1,279 localized+555 mCRPC=1,834 specimens
SV: 201 localized+101 mCRPC=302 specimens

### DNA Copy Number Aberrations

For the CPCG cohort, copy number aberrations (CNAs) were called from OncoScan FFPE v3 microarrays (n=382), as previously described⁷. For The Cancer Genome Atlas Prostate Adenocarcinoma (TCGA-PRAD; n=494) and Baca (n=53) cohorts, CNAs were called from Illumina SNP 6.0 microarrays. For the Taylor/MSKCC validation cohort (n=194), CNAs were called from Agilent 244K array comparative genomic hybridization (aCGH) microarrays. Overall, CNA calls were available for 1,279/1,289 localized patients. For mCRPC specimens from the Quigley study (n=101), CNAs were called from whole-genome sequencing (n=101) using CopyCat-generated BED files downloaded from the authors' website (available on the World Wide Web at davidquigley.com/prostate.html) with copy number status (‘gain’, ‘neutral’, or ‘loss’) assigned based on log₂ ratios according to the authors' guidelines¹⁸: for autosomal genes, gain: log 2 CN score ≥3, loss: log₂ CN score ≤1.65, neutral: 1.65≤log₂ CN score <3; for genes on chromosomes X and Y, gain: log 2 CN score ≥1.4; loss: log₂ CN score≤0.6; neutral: 0.6<log₂ CN score <1.4. For the Abida and Robinson mCRPC cohorts (n=454), CNAs were called from whole exome sequencing, as described²⁷. In all cases except CPCG and Quigley cohorts, CNA data were extracted from publicly available datasets via the CGDS-R package (v 1.3.0). In these cases, ‘shallow deletion’ and ‘deep deletion’ were pooled as ‘loss’ while ‘gain’ and ‘amplification’ were pooled as ‘gain’.

Percentage of the genome affected by a copy number alteration (PGA) was calculated as the number of bases affected by a CNA divided by the total number of bases in the genome, as previously reported^{7,30-32}. When comparing the PGA associated with the presence or absence of a specific CNA, an adjusted PGA was calculated by omitting the chromosome on which the specific gene is found.

### Single Nucleotide Variants

Coding single nucleotide variants (SNVs) in driver genes were called from whole-genome or whole exome sequencing data based on tumour-normal comparisons. Of the 1,289 localized prostate cancer patients, coding SNV data were available from 1.204 independent localized patient specimens (CPCG: 300, Barbieri: 109, Berger: 7, Baca: 53, TCGA: 494, Weischenfeldt: 11, and Gerhauser: 230) and 555 mCRPC patient specimens (Quigley: 101, SU2C: 454). As noted above, these numbers represent unique patients; where a given specimen was included in two or more studies, it was included only once in the current study. SNV data for all localized prostate cancer studies were downloaded from cBioPortal (via the CGDS-R package for R) or the ICGC Data Portal (dcc.icgc.org). For Quigley mCRPC specimens, VCF files were downloaded from the authors' website (available on the World Wide Web at davidquigley.com/prostate.html). For Robinson and Abida cohort mCRPC specimens, SNV calls were downloaded into R from cBioPortal using the CGDS-R package (v1.3.0). For each patient, the inventors extracted calls for missense (non-synonymous), nonsense (stop codon gained or stop codon lost), and splicing variants (spice donor or splice acceptor) within each gene analysed.

For non-coding SNVs in the Quigley cohort, hg19 coordinates from the CPCG study were re-mapped to GRCh38 (as described above).

The list of genes affected by SNVs in the current study is available in Table 2. Amongst the 43 genes evaluated for SNVs, 20 were included in a previous analysis of driver SNV enrichment in localized prostate cancer vs. mCRPC¹⁶, while the relative prevalence of 23 driver gene SNVs are assessed here for the first time.

### Structural Variants

Driver structural variants (SVs) in the CPCG cohort were previously reported⁷. For mCRPC specimens, SV calls from Manta³³ were downloaded from the authors' website (available on the World Wide Web at davidquigley.com/prostate.html). Overall, SV calls were available for 201 localized patients and 101 mCRPC patients. SVs included translocations and inversions, except where a specific SV type is specified for a given gene or locus. To assess SVs that were originally called from localized specimens in one megabase bins based on hg19 coordinates, these bins were re-mapped to GRCh38 using the NCBI Genome Remapping Service (available on the World Wide Web at ncbi.nlm.nih.gov/genome/tools/remap). For SVs at the PTEN locus, the inventors evaluated inter-chromosomal translocations and deletions separately from inversions within the chr10:89 Mbp bin (hg19), which regulate PTEN RNA abundance in localized prostate cancer⁷.

### RNA Abundance Data

RNA abundance data were available for 208 patients with clinical outcome data in the CPCG cohort²². For all cases, total RNA was extracted from tumour tissue sections, alternating with those used for whole-genome sequencing to minimize any effects of spatial heterogeneity. Total RNA (100 ng) was assayed using Affymetrix Human Transcriptome Array 2.0 and HuGene 2.0 ST microarrays, and RNA abundance calculated as previously reported^{7,10}. Samples were stratified as having “high” or “low” RNA abundance based on median dichotomization of log 2 abundance values.

The LHRI cohort RNA abundance was assessed from RNA-seq (n=47). Briefly, 200 ng of total RNA was used to construct a TruSeq strand specific library with the Ribo-Zero protocol (Illumina), and all samples were sequenced on a HiSeq2500 to a target depth of 50 million read pairs. Reads were mapped and mRNA abundance was quantified using the STAR aligner (v2.5.2a) against GRCh37 with Gencode Annotations (v24) lifted to GRCh37³⁴. Library normalization was performed using trimmed means of M-values (TMM) with the BioConductor package EdgeR (v3.12.1)³⁵. Samples were stratified as having “high” or “low” RNA abundance based on median dichotomization of log 2 abundance.

RNA abundance data for TCGA (RNA-seq; n=493) and Taylor/MSKCC (Affymetrix Human Exon 1.0 ST microarrays; n=216) were downloaded into R from cBioPortal (available on the World Wide Web at cBioPortal.org) using CGDS-R (v1.3.0). To take advantage of the increased statistical power from these larger cohorts, the inventors stratified samples into quartiles based on log 2 RNA abundance values.

Comparison of RNA abundance between groups was performed using the Spearman rank correlation coefficients and Mann-Whitney U tests. Gene Set Enrichment Analysis (GSEA) and Gene Ontology (GO) analyses were performed using the online Molecular Signatures Database tool from the Broad Institute (v7.0; Mar. 3, 2020; available on the World Wide Web at software.broadinstitute.org/gsea/msigdb/annotate.jsp) an G: Profiler g: GOSt (version e99_eg46_p14_f929183d, Jul. 20, 2020; available on the World Wide Web at biit.cs.ut.ee/gprofiler/).

### Comparison of Driver Aberration Prevalence

For the localized and mCRPC cohorts (as well as the two cohorts combined), the inventors calculated the proportion of samples harboring mutations in each driver gene (e.g. TP53) as well as the proportion of samples harboring each driver gene mutation type (e.g. TP53 SNVs, CNAs, and SVs). For each individual mutation class (CNA, SNV, or SV), the proportion (P_{MUT}) was calculated as:

$${P}_{MUT} = \frac{n umber of specimens with specific mutation}{n umber of specimens analysed for mutation class}$$

The inventors also calculated the proportion of specimens harboring more than one mutation class (i.e. CNA+SNV, CNA+SV, SNV+SV, and/or CNA+SNV+SV). 95% confidence intervals for the proportions of each mutational class (or combination of classes) were calculated as:

$$CI = p \pm 1.96 \sqrt{p ( 1 - p ) / n}$$

where p is the overall proportion for each mutation or combination of mutations for each gene and n is the number of samples analysed for that mutation class or combination of classes.

For each gene, the final proportion of cases harboring a mutation of any kind (P_{GENE}) was calculated as:

$${P}_{GENE} = ( {P}_{CNA} + {P}_{SNV} + {P}_{SV} ) - ( {P}_{CNA \& SNV} + {P}_{CNA \& SV} + {P}_{SNV \& SV} + {P}_{CNA \& SNV \& SV} )$$

where P is the proportion of cases harboring each stated mutation class (i.e. CNA, SNV, SV; as applicable to the specific gene). To account for unequal sample sizes for each mutation class (or combination of classes), the inventors calculated propagation of error for each mutation class (or combination of classes, as applicable) as:

$$P {E}_{class} = \sqrt{C {I}_{Upper}^{2} + C {I}_{Lower}^{2}}$$

Variance of propagation of error was calculated as:

$${\partial }_{GENE}^{2} = \sqrt{\begin{matrix}( P {E}_{CNA} + P {E}_{SNV} + P {E}_{SV} ) - ( P {E}_{CNA \& SNV} + \\ {PE}_{CNA \& SV} + P {E}_{SNV \& SV} + P {E}_{CNA \& SNV \& SV} )\end{matrix}}$$

Final 95% confidence intervals for each gene were calculated as above:

$$CI = p \pm 1.96 \sqrt{p ( 1 - p ) / n}$$

where p is the overall gene proportion and n is the number of samples analysed for that gene (Table 2).

The per patient overlap in available mutation data is shown in FIG. 5. Differences in proportion of localized and mCRPC patients (‘Δ proportion’) were calculated by subtracting the proportion of localized specimens harboring the specific aberration from the proportion of mCRPC specimens harboring the same aberration. Positive Δ proportion values indicate higher prevalence in mCRPC; negative Δ proportion values indicate enrichment in localized disease. To determine if specific mutations were statistically differentially prevalent between localized disease and mCRPC, the inventors used Fisher's Exact tests. For SNVs (coding and non-coding), the inventors corrected for global mutational burden as follows: first, the inventors used a binomial distribution to calculate the probability of observing no mutations in a given driver gene in a given sample, corrected for the size of the gene coding region (Mbp) and the mutational burden (SNVs per Mbp) in that sample (P_{NOMUT}); the probability of observing at least one mutation in a given gene was then calculated as: P_{MUT}=1−P_{NOMUT}. The inventors then performed a simulation of 100,000 samples of the binomial distribution, using P_{MUT} to weight the probability of mutation in each sample. A two-sided p-value was calculated as proportion of these simulations showing as great or greater simulated Δ proportion than observed Δ proportion.

The inventors corrected for CNA burden in a similar manner, using gene size and per sample CNA burden (i.e. proportion of the genome altered by a CNA×3×10⁹) to identify P_{MUT} (as above), which was then used to weight the sampling probability. The inventors then calculated a two-sided p-value for each driver CNA as half of the proportion of permutations showing as great or greater simulated Δ proportion than the observed Δ proportion (to account for the fact that a CNA can be either a gain or a loss).

Confidence intervals for Δ proportions were calculated using Yates' X² with Continuity Correction,

$$CI = {\hat{p}}_{1} - {\hat{p}}_{2} \pm 1.96 \sqrt{\frac{{\hat{p}}_{1} ( 1 - {\hat{p}}_{1} )}{m} + \frac{{\hat{p}}_{2} ( 1 - {\hat{p}}_{2} )}{n}} \pm \frac{1}{2} * ( \frac{1}{m} + \frac{1}{n} )$$

where p₁ is the proportion of mCRPC harboring the mutation, p₂ is the proportion in localized prostate cancer harboring the mutation, m is the number of mCRPC specimens tested, n is the number of localized prostate cancer specimens tested.

Adjusted Δ proportion was calculated as the difference between observed Δ proportion and expected Δ proportion; adjusted Δ proportion values >0 indicate a higher than expected proportion of mCRPC cases harboring the specific mutation while adjusted Δ proportion <0 indicates a higher than expected proportion in localized disease.

### Clinical Outcome

For patients undergoing RP, biochemical relapse (BCR) was defined according to American Urological Association (AUA) guidelines: two consecutive PSA values of >0.2 ng/mL over at least 6 months following treatment or initiation of salvage therapy. Patients who had initial PSA failure after RP and then underwent successful salvage RT (i.e. PSA<0.2 ng/mL in two consecutive tests within 6 months) were not classified as having BCR unless they subsequently met AUA (not Phoenix) conditions for BCR; in these cases, BCR was backdated to the time of initial post-RP PSA rise. For patients who underwent IGRT with curative intent, BCR was defined according to the Phoenix criteria³⁶: a PSA value of 2 ng/ml above PSA nadir or initiation of salvage hormone therapy. For the TCGA cohort, progression-free, disease-specific, and overall survival were used as reported by the consortium³⁷.

Biochemical relapse-free rate (bRFR), metastatic relapse-free rate (mRFR) and overall survival were calculated using the Kaplan-Meier method. Associations between mutations and outcome were assessed using log-rank or univariate Cox proportional hazards models, as appropriate. Adjustments for clinical factors [T-category (categorical; T1, T2a/b, or T2c), pre-treatment PSA, and diagnostic (i.e. biopsy) ISUP grade (categorical; Grade 1 vs. Grade 2 vs.≥Grade 3)] using multivariable Cox proportional hazards modeling. In all cases, the proportional hazards assumptions were verified graphically using Schoenfeld residuals. Log-rank tests were used when the proportional hazards assumptions were violated.

### Prognostic Signature Scores

Fraser signature scores were calculated based on a modified version of the six feature signature reported in Fraser et al⁷. Briefly, MYC gain, ATM SNVs, TCERGL1 hypomethylation, ACTL6B hypermethylation, chr7:61 Mbp inter-chromosomal translocations, and clinical T category were scored for each patient; CNAs, SNVs, and SVs were scored as absent (0) or present (1); probe-based methylation β-values were median dichotomized and patients scored as being either above or below the median; clinical T category was scored as cT1 or cT2a/b (0) or cT2c (1). For multivariable analyses, a signature score was derived based on the sum of the six features. For visualization, scores were median dichotomized and patients assigned to either “Signature High” or “Signature Low” bins.

Cell Cycle Progression/Prolaris scores were approximated as previously reported^{38,39}. Briefly, the mean abundance of the 31 CCP genes was normalized to the mean abundance of 15 housekeeping genes to yield a CCP score, as previously reported³⁹. Outcomes analyses were performed using CCP as a continuous variable.

### Statistical Testing and Data Visualization

All statistical analyses were performed using R statistical software (v3.5.2) with the following packages: BoutrosLab.plotting.general (v5.9.2)⁴⁰, VennDiagram (v1.6.20)⁴¹, CGDS-R (v1.3.0), survival (v2.43-3), ggplot2 (v3.1.0), easyGgplot2 (v1.0), survminer (v0.4.3), forestplot (v.1.7.2), factorial2×2 (v0.2.0), cowplot (v1.1.0) and tidyverse (v1.2.1). All tests of statistical significance were two-sided. P-values were corrected for multiple comparisons using the Benjamini-Hochberg False Discovery Rate (FDR) method or the Bonferroni method, as noted. For Analysis of Variance (ANOVA), between group significance was evaluated using Tukey post hoc tests.

TABLE 1
Multivariable Cox Proportional Hazards Model of ZNRF3 Loss
on Metastatic Relapse, Corrected for Clinical Prognostic
Factors, Adjusted PGA, and IDC-P/CA

| | | | | 95% | |
|---|---|---|---|---|---|
| | | | Hazard | Confidence | |
| Variable | Level | N | Ratio | Interval | P-Value |
| ZNRF3 Loss | | | | | |
| | Neutral | 298 | — | — | — |
| | Loss | 28 | 3.53 | 1.47-8.47 | 4.72E−03 |
| PSA | Continuous | 326 | 1.05 | 0.96-1.14 | 0.31 |
| Gleason Score | | | | | |
| | 3 + 3 | 54 | — | — | — |
| | 3 + 4 | 190 | 4.29 | 0.53-34.6 | 0.17 |
| | 4 + 3 | 73 | 9.62 | 1.15-80.3 | 0.036 |
| | 4 + 4 and | 9 | 5.33 | 0.31-93.0 | 0.25 |
| | above | | | | |
| Clinical T | | | | | |
| Category | | | | | |
| | cT1 | 157 | — | — | — |
| | cT2a/b | 160 | 1.63 | 0.68-3.94 | 0.27 |
| | cT2c | 9 | 2.47 | 0.29-21.4 | 0.41 |
| Adjusted PGA | Continuous | 326 | 1.09 | 0.47-2.53 | 0.84 |
| IDC-P/CA | | | | | |
| | Absent | 222 | — | — | — |
| | Present | 104 | 1.58 | 0.67-3.57 | 0.27 |

TABLE 2
Gene-Wise Driver Mutation Summary

| gene.id | Mutation.Types | n.gene | gene.proportion | CI.Upper | CI.Lower |
|---|---|---|---|---|---|
| NKX3-1 | CNA + SV | 1838 | 0.572172431 | 0.6133121 | 0.53103276 |
| ERG | SV ONLY | 302 | 0.400662252 | 0.48596163 | 0.31536287 |
| MYC | CNA + SV | 1838 | 0.363418721 | 0.39624502 | 0.33059242 |
| RB1 | CNA + SNV + SV | 1844 | 0.336367227 | 0.36787791 | 0.30485655 |
| TP53 | CNA + SNV + SV | 1844 | 0.329984736 | 0.36081924 | 0.29915024 |
| PTEN | CNA + SNV + SV | 1844 | 0.28835956 | 0.31699963 | 0.25971949 |
| CDH1 | CNA ONLY | 1834 | 0.268266085 | 0.29649638 | 0.24003579 |
| PRKDC | CNA + SNV + SV | 1844 | 0.2594323 | 0.28718467 | 0.23167993 |
| ZFHX3 | CNA + SNV | 1844 | 0.258854307 | 0.28651625 | 0.23119237 |
| CHD1 | CNA + SV | 1838 | 0.258660908 | 0.2863455 | 0.23097631 |
| ETV1 | CNA + SV | 1838 | 0.251572577 | 0.27890246 | 0.22424269 |
| AR | CNA + SNV | 1844 | 0.24015033 | 0.26679725 | 0.21350341 |
| ETV5 | CNA + SV | 1838 | 0.185665177 | 0.2094934 | 0.16183695 |
| NCOR1 | CNA + SNV + SV | 1844 | 0.18071558 | 0.20392261 | 0.15750855 |
| ZNRF3 | CNA + SV | 1838 | 0.176901356 | 0.19982562 | 0.15397709 |
| BRCA2 | CNA + SNV | 1844 | 0.16787849 | 0.19017978 | 0.1455772 |
| CCND1 | CNA ONLY | 1834 | 0.155943293 | 0.17749697 | 0.13438962 |
| BRAF | CNA + SNV + SV | 1844 | 0.154869603 | 0.1763705 | 0.1333687 |
| FOXA1 | CNA + SNV + SV | 1844 | 0.130478959 | 0.15023578 | 0.11072214 |
| ZBTB16 | CNA ONLY | 1834 | 0.126499455 | 0.14592698 | 0.10707193 |
| chr21:42 Mbp | SV ONLY | 302 | 0.122516556 | 0.17049811 | 0.07453501 |
| APC | CNA + SNV | 1844 | 0.118974079 | 0.13777336 | 0.1001748 |
| ATM | CNA + SNV + SV | 1844 | 0.111913158 | 0.13016143 | 0.09366488 |
| MSH2 | CNA + SNV + SV | 1844 | 0.111551408 | 0.13023405 | 0.09286877 |
| CDKN2A | CNA + SNV + SV | 1844 | 0.109339032 | 0.12781959 | 0.09085847 |
| AKT1 | CNA + SNV | 1844 | 0.096702262 | 0.11368218 | 0.07972234 |
| CTNNB1 | CNA + SNV | 1844 | 0.091178285 | 0.10766759 | 0.07468898 |
| TTN | SNV ONLY | 1759 | 0.088118249 | 0.10470801 | 0.07152849 |
| chr10:89 Mbp | SV ONLY | 302 | 0.082781457 | 0.1226649 | 0.04289801 |
| chr7:61 Mbp | SV ONLY | 302 | 0.082781457 | 0.1226649 | 0.04289801 |
| ASXL2 | CNA + SNV + SV | 1844 | 0.082596482 | 0.09835779 | 0.06683518 |
| SPOP | SNV ONLY | 1759 | 0.076748152 | 0.09224453 | 0.06125177 |
| HDAC4 | CNA + SV | 1838 | 0.071280522 | 0.08645875 | 0.0561023 |
| chr17:25 Mbp | SV ONLY | 302 | 0.069536424 | 0.10632242 | 0.03275043 |
| BRCA1 | CNA + SNV | 1844 | 0.066266819 | 0.08035697 | 0.05217666 |
| CDK12 | CNA ONLY | 1834 | 0.065976009 | 0.08005851 | 0.05189351 |
| KDM6A | CNA + SNV + SV | 1844 | 0.065152101 | 0.07973816 | 0.05056604 |
| MUC16 | SNV ONLY | 1759 | 0.062535532 | 0.07654565 | 0.04852541 |
| MSH6 | CNA + SNV | 1844 | 0.056875306 | 0.06995403 | 0.04379658 |
| chr3:129 Mbp | SV ONLY | 302 | 0.056291391 | 0.08968929 | 0.02289349 |
| chr3:125 Mbp | SV ONLY | 302 | 0.052980132 | 0.08547474 | 0.02048552 |
| chr4:148 Mbp | SV ONLY | 302 | 0.049668874 | 0.08123373 | 0.01810402 |
| chr6:97 Mbp | SV ONLY | 302 | 0.046357616 | 0.07696371 | 0.01575152 |
| ETV4 | SV ONLY | 302 | 0.043046358 | 0.07266171 | 0.013431 |
| CSMD3 | SNV ONLY | 1759 | 0.039795338 | 0.05102515 | 0.02856553 |
| SYNE1 | SNV ONLY | 1759 | 0.036952814 | 0.04778496 | 0.02612066 |
| PIK3CA | SV ONLY | 302 | 0.036423841 | 0.06394689 | 0.00890079 |
| KMT2C | SNV ONLY | 1759 | 0.031267766 | 0.04125713 | 0.02127841 |
| chr7:145019604 | SNV ONLY | 302 | 0.029801325 | 0.05505065 | 0.004552 |
| AHNAK2 | SNV ONLY | 1759 | 0.028425242 | 0.03796526 | 0.01888522 |
| MED12 | SNV + SV | 1759 | 0.027457654 | 0.03766532 | 0.01724999 |
| RYR2 | SNV ONLY | 1759 | 0.027288232 | 0.03664246 | 0.01793401 |
| chr22:31826396 | SNV ONLY | 302 | 0.026490066 | 0.05051679 | 0.00246334 |
| chr4:39684557 | SNV ONLY | 302 | 0.026490066 | 0.05051679 | 0.00246334 |
| ERCC2 | SNV + SV | 1759 | 0.024884322 | 0.03471686 | 0.01505178 |
| KMT2D | SNV ONLY | 1759 | 0.023877203 | 0.03265039 | 0.01510401 |
| chr13:58055742 | SNV ONLY | 302 | 0.023178808 | 0.04591193 | 0.00044569 |
| chr16:64910033 | SNV ONLY | 302 | 0.023178808 | 0.04591193 | 0.00044569 |
| chr2:131394079 | SNV ONLY | 302 | 0.023178808 | 0.04591193 | 0.00044569 |
| FAT3 | SNV ONLY | 1759 | 0.022171688 | 0.03063936 | 0.01370401 |
| SPTA1 | SNV ONLY | 1759 | 0.018760659 | 0.02658141 | 0.01093991 |
| AXL | SV ONLY | 302 | 0.016556291 | 0.03642172 | 0 |
| PCDH15 | SNV ONLY | 1759 | 0.01534963 | 0.02246447 | 0.00823479 |
| NCOR2 | SNV ONLY | 1759 | 0.013075611 | 0.01967766 | 0.00647357 |
| PRB4 | SNV ONLY | 1759 | 0.012507106 | 0.01897453 | 0.00603968 |
| GNAS | SNV ONLY | 1759 | 0.011938601 | 0.0182685 | 0.0056087 |
| QRICH2 | SNV ONLY | 1759 | 0.009664582 | 0.01541103 | 0.00391814 |
| ADAMTS20 | SNV ONLY | 1759 | 0.008527572 | 0.01395834 | 0.00309681 |
| TPTE | SNV ONLY | 1759 | 0.007959068 | 0.01322472 | 0.00269342 |
| IDH1 | SNV ONLY | 1759 | 0.003411029 | 0.00708307 | 0 |
| HRAS | SNV ONLY | 1759 | 0.002842524 | 0.0062583 | 0 |
| MLH1 | SNV ONLY | 1759 | 0.002842524 | 0.0062583 | 0 |

TABLE 3
Proportion of Driver Mutations in Localized and Metastatic Prostate Cancer Specimens

| gene.id | type | Abb.Code | Abberation.Type | n.mets | events.mets | n.localized | events.localized |
|---|---|---|---|---|---|---|---|
| NKX3-1 | CNA | NKX3-1 CNA | Genomic Loss | 555 | 375 | 1279 | 644 |
| ERG | SV | ERG SV | SV | 101 | 43 | 201 | 78 |
| PTEN | SV | PTEN SV | SV | 101 | 22 | 201 | 45 |
| MYC | CNA | MYC CNA | Genomic Gain | 555 | 357 | 1279 | 267 |
| CDH1 | CNA | CDH1 CNA | Genomic Loss | 555 | 236 | 1279 | 256 |
| RB1 | SV | RB1 SV | SV | 101 | 4 | 201 | 32 |
| CHD1 | SV | CHD1 SV | SV | 101 | 2 | 201 | 32 |
| CHD1 | CNA | CHD1 CNA | Genomic Loss | 555 | 156 | 1279 | 203 |
| ETV1 | CNA | ETV1 CNA | Genomic Gain | 555 | 168 | 1279 | 178 |
| RB1 | CNA | RB1 CNA | Genomic Loss | 555 | 343 | 1279 | 175 |
| PTEN | CNA | PTEN CNA | Genomic Loss | 555 | 289 | 1279 | 165 |
| ZFHX3 | CNA | ZFHX3 CNA | Genomic Loss | 555 | 285 | 1279 | 163 |
| TP53 | CNA | TP53 CNA | Genomic Loss | 555 | 336 | 1279 | 156 |
| NKX3-1 | SV | NKX3-1 SV | SV | 101 | 2 | 201 | 23 |
| CCND1 | CNA | CCND1 CNA | Genomic Gain | 555 | 151 | 1279 | 135 |
| ZNRF3 | CNA | ZNRF3 CNA | Genomic Loss | 555 | 166 | 1279 | 122 |
| ETV5 | CNA | ETV5 CNA | Genomic Gain | 555 | 181 | 1279 | 117 |
| PRKDC | SV | PRKDC SV | SV | 101 | 5 | 201 | 18 |
| SPOP | SNV | SPOP SNV | Non-synonymous | 555 | 32 | 1204 | 103 |
| chr21:42 | SV | chr21:42 | Inter-chromosomal | 101 | 20 | 201 | 17 |
| Mbp | | Mbp SV | translocation | | | | |
| ZBTB16 | CNA | ZBTB16 CNA | Genomic Loss | 555 | 127 | 1279 | 105 |
| chr3:129 | SV | chr3:129 | Inversion | 101 | 2 | 201 | 15 |
| Mbp | | Mbp SV | | | | | |
| chr3:125 | SV | chr3:125 | Inversion | 101 | 1 | 201 | 15 |
| Mbp | | Mbp SV | | | | | |
| chr6:97 | SV | chr6:97 | Inter-chromosomal | 101 | 1 | 201 | 13 |
| Mbp | | Mbp SV | translocation | | | | |
| PRKDC | CNA | PRKDC CNA | Genomic Gain | 555 | 247 | 1279 | 77 |
| ETV1 | SV | ETV1 SV | SV | 101 | 12 | 201 | 12 |
| chr4:148 | SV | chr4:148 | Inter-chromosomal | 101 | 3 | 201 | 12 |
| Mbp | | Mbp SV | translocation | | | | |
| CDK12 | CNA | CDK12 CNA | Genomic Loss | 555 | 47 | 1279 | 74 |
| BRCA2 | CNA | BRCA2 CNA | Genomic Loss | 555 | 219 | 1279 | 72 |
| chr10:89 | SV | chr10:89 | Inversion | 101 | 14 | 201 | 11 |
| Mbp | | Mbp SV | | | | | |
| TP53 | SV | TP53 SV | SV | 101 | 13 | 201 | 11 |
| chr17:25 | SV | chr17:25 | Inter-chromosomal | 101 | 10 | 201 | 11 |
| Mbp | | Mbp SV | translocation | | | | |
| MSH2 | SNV | MSH2 SNV | Non-synonymous | 555 | 8 | 1204 | 64 |
| ATM | SV | ATM SV | SV | 101 | 6 | 201 | 10 |
| PIK3CA | SV | PIK3CA SV | SV | 101 | 1 | 201 | 10 |
| NCOR1 | CNA | NCOR1 CNA | Genomic Loss | 555 | 199 | 1279 | 59 |
| chr7:145019604 | ncSNV | chr7:145019604 | Non-coding SNV | 101 | 0 | 201 | 9 |
| | | SNV | | | | | |
| ASXL2 | SV | ASXL2 SV | SV | 101 | 7 | 201 | 9 |
| MYC | SV | MYC SV | SV | 101 | 2 | 201 | 9 |
| APC | CNA | APC CNA | Genomic Loss | 555 | 139 | 1279 | 57 |
| CDKN2A | CNA | CDKN2A CNA | Genomic Loss | 555 | 107 | 1279 | 56 |
| TTN | SNV | TTN SNV | Non-synonymous | 555 | 103 | 1204 | 52 |
| BRCA1 | CNA | BRCA1 CNA | Genomic Loss | 555 | 58 | 1279 | 55 |
| AKT1 | CNA | AKT1 CNA | Genomic Gain | 555 | 117 | 1279 | 54 |
| TP53 | SNV | TP53 SNV | Non-synonymous | 555 | 209 | 1204 | 49 |
| chr22:31826396 | ncSNV | chr22:31826396 | Non-coding SNV | 101 | 0 | 201 | 8 |
| | | SNV | | | | | |
| chr7:61 | SV | chr7:61 | Inter-chromosomal | 101 | 17 | 201 | 8 |
| Mbp | | Mbp SV | translocation | | | | |
| BRAF | SV | BRAF SV | SV | 101 | 2 | 201 | 8 |
| BRAF | CNA | BRAF CNA | Genomic Gain | 555 | 184 | 1279 | 45 |
| chr4:39684557 | ncSNV | chr4:39684557 | Non-coding SNV | 101 | 1 | 201 | 7 |
| | | SNV | | | | | |
| chr13:58055742 | ncSNV | chr13:58055742 | Non-coding SNV | 101 | 0 | 201 | 7 |
| | | SNV | | | | | |
| chr16:64910033 | ncSNV | chr16:64910033 | Non-coding SNV | 101 | 0 | 201 | 7 |
| | | SNV | | | | | |
| chr2:131394079 | ncSNV | chr2:131394079 | Non-coding SNV | 101 | 0 | 201 | 7 |
| | | SNV | | | | | |
| ZNRF3 | SV | ZNRF3 SV | SV | 101 | 11 | 201 | 7 |
| ETV4 | SV | ETV4 SV | SV | 101 | 6 | 201 | 7 |
| NCOR1 | SV | NCOR1 SV | SV | 101 | 5 | 201 | 7 |
| ERCC2 | SV | ERCC2 SV | SV | 101 | 1 | 201 | 6 |
| MSH2 | SV | MSH2 SV | SV | 101 | 1 | 201 | 6 |
| KDM6A | SV | KDM6A SV | SV | 101 | 6 | 201 | 5 |
| ETV5 | SV | ETV5 SV | SV | 101 | 2 | 201 | 5 |
| ATM | CNA | ATM CNA | Genomic Loss | 555 | 106 | 1279 | 30 |
| RB1 | SNV | RB1 SNV | Non-synonymous | 555 | 21 | 1204 | 26 |
| MUC16 | SNV | MUC16 SNV | Non-synonymous | 555 | 86 | 1204 | 24 |
| CDKN2A | SV | CDKN2A SV | SV | 101 | 2 | 201 | 4 |
| AXL | SV | AXL SV | SV | 101 | 1 | 201 | 4 |
| HDAC4 | CNA | HDAC4 CNA | Genomic Gain | 555 | 47 | 1279 | 23 |
| MSH2 | CNA | MSH2 CNA | Genomic Loss | 555 | 66 | 1279 | 22 |
| CSMD3 | SNV | CSMD3 SNV | Non-synonymous | 555 | 50 | 1204 | 20 |
| MSH6 | CNA | MSH6 CNA | Genomic Loss | 555 | 78 | 1279 | 21 |
| FOXA1 | SV | FOXA1 SV | SV | 101 | 6 | 201 | 3 |
| MED12 | SV | MED12 SV | SV | 101 | 1 | 201 | 3 |
| CTNNB1 | CNA | CTNNB1 CNA | Genomic Gain | 555 | 124 | 1279 | 19 |
| PTEN | SNV | PTEN SNV | Non-synonymous | 555 | 38 | 1204 | 17 |
| FOXA1 | SNV | FOXA1 SNV | Non-synonymous | 555 | 48 | 1204 | 16 |
| RYR2 | SNV | RYR2 SNV | Non-synonymous | 555 | 32 | 1204 | 16 |
| PRB4 | SNV | PRB4 SNV | Non-synonymous | 555 | 8 | 1204 | 14 |
| ASXL2 | CNA | ASXL2 CNA | Genomic Loss | 555 | 52 | 1279 | 14 |
| SPTA1 | SNV | SPTA1 SNV | Non-synonymous | 555 | 21 | 1204 | 12 |
| MED12 | SNV | MED12 SNV | Non-synonymous | 555 | 13 | 1204 | 12 |
| HDAC4 | SV | HDAC4 SV | SV | 101 | 8 | 201 | 2 |
| FOXA1 | CNA | FOXA1 CNA | Genomic Gain | 555 | 130 | 1279 | 11 |
| SYNE1 | SNV | SYNE1 SNV | Non-synonymous | 555 | 55 | 1204 | 10 |
| ATM | SNV | ATM SNV | Non-synonymous | 555 | 29 | 1204 | 10 |
| FAT3 | SNV | FAT3 SNV | Non-synonymous | 555 | 29 | 1204 | 10 |
| ZFHX3 | SNV | ZFHX3 SNV | Non-synonymous | 555 | 29 | 1204 | 10 |
| NCOR2 | SNV | NCOR2 SNV | Non-synonymous | 555 | 13 | 1204 | 10 |
| KDM6A | CNA | KDM6A CNA | Genomic Loss | 555 | 27 | 1279 | 10 |
| PRKDC | SNV | PRKDC SNV | Non-synonymous | 555 | 20 | 1204 | 9 |
| PCDH15 | SNV | PCDH15 SNV | Non-synonymous | 555 | 18 | 1204 | 9 |
| KMT2C | SNV | KMT2C SNV | Non-synonymous | 555 | 47 | 1204 | 8 |
| AHNAK2 | SNV | AHNAK2 SNV | Non-synonymous | 555 | 45 | 1204 | 5 |
| APC | SNV | APC SNV | Non-synonymous | 555 | 43 | 1204 | 5 |
| GNAS | SNV | GNAS SNV | Non-synonymous | 555 | 16 | 1204 | 5 |
| KDM6A | SNV | KDM6A SNV | Non-synonymous | 555 | 11 | 1204 | 5 |
| TPTE | SNV | TPTE SNV | Non-synonymous | 555 | 9 | 1204 | 5 |
| IDH1 | SNV | IDH1 SNV | Non-synonymous | 555 | 1 | 1204 | 5 |
| BRAF | SNV | BRAF SNV | Non-synonymous | 555 | 6 | 1204 | 4 |
| CTNNB1 | SNV | CTNNB1 SNV | Non-synonymous | 555 | 25 | 1204 | 3 |
| NCOR1 | SNV | NCOR1 SNV | Non-synonymous | 555 | 13 | 1204 | 3 |
| ADAMTS20 | SNV | ADAMTS20 | Non-synonymous | 555 | 12 | 1204 | 3 |
| | | SNV | | | | | |
| KMT2D | SNV | KMT2D SNV | Non-synonymous | 555 | 40 | 1204 | 2 |
| QRICH2 | SNV | QRICH2 SNV | Non-synonymous | 555 | 15 | 1204 | 2 |
| BRCA1 | SNV | BRCA1 SNV | Non-synonymous | 555 | 10 | 1204 | 2 |
| AKT1 | SNV | AKT1 SNV | Non-synonymous | 555 | 6 | 1204 | 2 |
| AR | CNA | AR CNA | Genomic Gain | 555 | 395 | 1279 | 2 |
| ASXL2 | SNV | ASXL2 SNV | Non-synonymous | 555 | 13 | 1204 | 1 |
| MLH1 | SNV | MLH1 SNV | Non-synonymous | 555 | 4 | 1204 | 1 |
| AF | SNV | AR SNV | Non-synonymous | 555 | 76 | 1204 | 0 |
| BRCA2 | SNV | BRCA2 SNV | Non-synonymous | 555 | 41 | 1204 | 0 |
| MSH6 | SNV | MSH6 SNV | Non-synonymous | 555 | 7 | 1204 | 0 |
| HRAS | SNV | HRAS SNV | Non-synonymous | 555 | 5 | 1204 | 0 |
| ERCC2 | SNV | ERCC2 SNV | Non-synonymous | 555 | 3 | 1204 | 0 |
| CDKN2A | SNV | CDKN2A SNV | Non-synonymous | 555 | 2 | 1204 | 0 |

TABLE 4
Driver Gene Mutations With Significantly Difference Prevalence in
mCRPC than Expected and Present in at Least 5% of Localized Cancers

| gene.id | type | Abb.Code | Abberation.Type | n.mets | events.mets | n.localized | events.localized |
|---|---|---|---|---|---|---|---|
| BRCA2 | CNA | BRCA2 CNA | Genomic Loss | 555 | 219 | 1279 | 72 |
| CCND1 | CNA | CCND1 CNA | Genomic Gain | 555 | 151 | 1279 | 135 |
| CDH1 | CNA | CDH1 CNA | Genomic Loss | 555 | 236 | 1279 | 256 |
| CDK12 | CNA | CDK12 CNA | Genomic Loss | 555 | 47 | 1279 | 74 |
| CHD1 | CNA | CHD1 CNA | Genomic Loss | 555 | 156 | 1279 | 203 |
| CHD1 | SV | CHD1 SV | SV | 101 | 2 | 201 | 32 |
| chr10:89 | SV | chr10:89 | Inversion | 101 | 14 | 201 | 11 |
| Mbp | | Mbp SV | | | | | |
| chr21:42 | SV | chr21:42 | Inter-chromosomal | 101 | 20 | 201 | 17 |
| Mbp | | Mbp SV | translocation | | | | |
| chr3:125 | SV | chr3:125 | Inversion | 101 | 1 | 201 | 15 |
| Mbp | | Mbp SV | | | | | |
| ETV1 | CNA | ETV1 CNA | Genomic Gain | 555 | 168 | 1279 | 178 |
| ETV5 | CNA | ETV5 CNA | Genomic Gain | 555 | 181 | 1279 | 117 |
| MSH2 | SNV | MSH2 SNV | Non-synonymous | 555 | 8 | 1204 | 64 |
| MYC | CNA | MYC CNA | Genomic Gain | 555 | 357 | 1279 | 267 |
| NKX3-1 | CNA | NKX3-1 | Genomic Loss | 555 | 375 | 1279 | 644 |
| | | CNA | | | | | |
| NKX3-1 | SV | NKX3-1 SV | SV | 101 | 2 | 201 | 23 |
| PRKDC | CNA | PRKDC CNA | Genomic Gain | 555 | 247 | 1279 | 77 |
| PTEN | CNA | PTEN CNA | Genomic Loss | 555 | 289 | 1279 | 165 |
| RB1 | CNA | RB1 CNA | Genomic Loss | 555 | 343 | 1279 | 175 |
| RB1 | SV | RB1 SV | SV | 101 | 4 | 201 | 32 |
| SPOP | SNV | SPOP SNV | Non-synonymous | 555 | 32 | 1204 | 103 |
| TP53 | CNA | TP53 CNA | Genomic Loss | 555 | 336 | 1279 | 156 |
| ZBTB16 | CNA | ZBTB16 | Genomic Loss | 555 | 127 | 1279 | 105 |
| | | CNA | | | | | |
| ZFHX3 | CNA | ZFHX3 CNA | Genomic Loss | 555 | 285 | 1279 | 163 |
| ZNRF3 | CNA | ZNRF3 CNA | Genomic Loss | 555 | 166 | 1279 | 122 |

TABLE 5
Univariable Cox Proportional Hazards Modeling (Metastasis-Free Survival) of Driver
Mutations Enriched in mCRPC and Present in at Least 5% of Localized Cancers

| | | Number of | Number of | | | | | | |
|---|---|---|---|---|---|---|---|---|---|
| Gene | | Patients with | Patients with | | HR | HR | wald | p | q |
| Locus | type | Molecular Data | Mutation | HR | Upper | Lower | test | value | value |
| MYC | CNA | 376 | 67 | 4.23 | 2.2 | 8.3 | 18 | 2.70E−05 | 6.00E−04 |
| CCND1 | CNA | 376 | 80 | 3.89 | 2 | 7.7 | 15 | 9.70E−05 | 7.60E−04 |
| ZNRF3 | CNA | 376 | 27 | 4.58 | 2.1 | 9.9 | 15 | 1.00E−04 | 7.67E−04 |
| PRKDC | CNA | 376 | 48 | 3.33 | 1.6 | 7 | 10 | 0.0014 | 8.05E−03 |
| CDK12 | CNA | 376 | 18 | 3.31 | 8.54 | 1.28 | 6.12 | 1.34E−02 | 0.062 |
| ETV5 | CNA | 376 | 28 | 2.37 | 0.92 | 6.1 | 3.2 | 0.075 | 0.25 |
| TP53 | CNA | 376 | 89 | 1.84 | 0.94 | 3.6 | 3.2 | 0.076 | 0.25 |
| ETV1 | CNA | 376 | 47 | 2.01 | 0.83 | 4.9 | 2.4 | 0.12 | 0.307 |
| CHD1 | CNA | 376 | 54 | 1.92 | 0.87 | 4.2 | 2.6 | 0.11 | 0.307 |
| ZFHX3 | CNA | 376 | 99 | 1.59 | 0.8 | 3.2 | 1.7 | 0.19 | 0.437 |
| PTEN | CNA | 376 | 115 | 1.47 | 0.75 | 2.9 | 1.2 | 0.27 | 0.565 |
| ZBTB16 | CNA | 376 | 30 | 1.56 | 0.55 | 4.4 | 0.71 | 0.4 | 0.767 |
| chr17:25Mbp | SV | 201 | 11 | 1.73 | 0.39 | 7.7 | 0.52 | 0.47 | 0.772 |
| BRCA2 | CNA | 376 | 46 | 1.41 | 0.55 | 3.7 | 0.51 | 0.47 | 0.772 |
| CDH1 | CNA | 376 | 60 | 0.82 | 0.32 | 2.1 | 0.16 | 0.69 | 0.934 |
| ERG | SV | 201 | 78 | 0.8 | 0.28 | 2.3 | 0.17 | 0.68 | 0.934 |
| chr21:42Mbp | SV | 201 | 17 | 0.62 | 0.082 | 4.8 | 0.2 | 0.65 | 0.934 |
| ETV1 | SV | 376 | 12 | 1.23 | 0.16 | 9.4 | 0.04 | 0.84 | 0.953 |
| SPOP | SNV | 294 | 32 | 1.1 | 0.33 | 3.7 | 0.03 | 0.87 | 0.953 |
| RB1 | CNA | 376 | 114 | 0.89 | 0.43 | 1.9 | 0.08 | 0.78 | 0.953 |
| chr10:89Mbp | SV | 201 | 11 | 0.86 | 0.24 | 3.1 | 0.05 | 0.83 | 0.953 |
| TP53 | SV | 201 | 11 | 1.34 | 0 | Inf | 0 | 1 | 1 |
| NKX3-1 | CNA | 376 | 188 | 1.01 | 0.52 | 2 | 0 | 0.99 | 1 |
| MSH2 | SNV | 294 | 0 | NA | NA | NA | NA | NA | NA |

TABLE 6A
Progression-Free Survival in TCGA patients,
Stratified by Driver CNAs

| | | | |
|---|---|---|---|
| Cohort | CNA | HR (95% CI) | Q-Value |

Univariable CoxPH Analysis

| | | | |
|---|---|---|---|
| TCGA PRAD | MYC Gain | 1.53 (1.01-2.32) | 4.72E−02 |
| | CCND1 Gain | 1.84 (1.04-3.25) | 0.0361 |
| | PRKDC Gain | 1.26 (0.80-1.99) | 0.319 |
| | ZNRF3 Loss | 2.56 (1.56-4.21) | 2.06E−04 |

Multivariable CoxPH Analysis

| | | | |
|---|---|---|---|
| TCGA PRAD | MYC Gain | 1.56 (0.833-3.06) | 1.59E−01 |
| | CCND1 Gain | 1.20 (0.629-2.28) | 0.582 |
| | PRKDC Gain | 0.786 (0.402-1.54) | 0.483 |
| | ZNRF3 Loss | 2.34 (1.39-3.94) | 1.41E−03 |

TABLE 6B
Biochemical Relapse in Taylor cohort patients, Stratified by Driver CNAs

| CNA | HR (95% CI) | Q-Value | Test |
|---|---|---|---|
| MYC Gain | 12.4 (4.03-38.0) | 3.18E−05 | CoxPH; Wald |
| CCND1 Gain | 11.1 (2.42-50.8) | 1.94E−03 | CoxPH; Wald |
| PRKDC Gain | 11.1 (3.72-33.2) | 3.18E−05 | CoxPH; Wald |
| ZNRF3 Loss | 7.67 (2.52-23.3) | 4.40E−04 | CoxPH; Wald |

TABLE 6C
Associations Between Driver CNAs and Metastatic
Relapse in Taylor cohort patients

| CNA | OR (95% CI) | Q-Value | Test |
|---|---|---|---|
| MYC Gain | 4.46 (1.98-10.0) | 0.00132 | Fisher's Exact test |
| CCND1 Gain | 16.2 (1.84-143) | 0.00456 | Fisher's Exact test |
| PRKDC Gain | 4.48 (−1.82-11.0) | 0.0016 | Fisher's Exact test |
| ZNRF3 Loss | 5.78 (2.07-16.1) | 0.0016 | Fisher's Exact test |

TABLE 7
Univariable Cox Proportional Hazards Models of Clinical Prognostic
Factors on Metastatic Relapse, CPC-GENE Cohort

| | | Number | | | p-value |
|---|---|---|---|---|---|
| | | of | Hazard | | (Wald |
| Factor | Level | Patients | Ratio | 95% CI | Test) |
| ISUP Grade | 1 | 65 | Reference | — | — |
| | 2 | 221 | 6.04 | 0.809-45.2 | 0.08 |
| | 3 | 80 | 17 | 2.21-131 | 0.0065 |
| | 4 and above | 10 | 6.04 | 0.371-98.5 | 0.207 |
| Clinical | cT1 | 179 | Reference | — | — |
| T-Category | cT2a/b | 187 | 2.25 | 1.07-4.70 | 0.032 |
| | cT2c | 10 | 2.5 | 0.318-19.6 | 0.384 |
| PSA | Continuous | 376 | 1.06 | 1.01-1.12 | 0.014 |
| (Continuous) | | | | | |
| PSA | <10 ng/ml | 274 | Reference | | |
| (Discrete) | ≥10 ng/ml | 102 | 2.07 | 1.06-4.04 | 0.034 |

TABLE 8
Multivariable Cox Proportional Hazards Analysis of ZNRF3 Loss and
the 6-Feature Fraser Signature effects on Biochemical and Metastatic
Relapse, controlled for ISUP grade, PSA, and clinical T category

| | | | | | |
|---|---|---|---|---|---|
| | | | | 95% | |
| | | | Hazard | Confidence | |
| Variable | Level | N | Ratio | Interval | P-Value |

BIOCHEMICAL RELAPSE

| | | | | | |
|---|---|---|---|---|---|
| Fraser Signature | Continuous | 375 | 1.59 | 1.37-1.85 | 1.29E−09 |
| ZNRF3 CNA | | | | | |
| | Diploid | 344 | — | — | — |
| | Loss | 31 | 1.77 | 1.06-2.98 | 3.00E−02 |
| PSA | Continuous | 375 | 1.04 | 1.00-1.07 | 0.031 |
| ISUP Grade | | | | | |
| | 1 | 65 | — | — | — |
| | 2 | 180 | 0.81 | 0.48-1.36 | 0.42 |
| | 3 and | 90 | 1.43 | 0.82-2.49 | 0.209 |
| | above | | | | |

METASTATIC RELAPSE

| | | | | | |
|---|---|---|---|---|---|
| Fraser Signature | Continuous | 375 | 1.58 | 1.17-2.12 | 2.69E−03 |
| ZNRF3 CNA | | | | | |
| | Diploid | 344 | — | — | — |
| | Loss | 31 | 2.86 | 1.29-6.34 | 0.019 |
| PSA | Continuous | 375 | 1.07 | 0.999-1.15 | 0.055 |
| ISUP Grade | | | | | |
| | 1 | 65 | — | — | — |
| | 2 | 180 | 5.95 | 0.761-46.6 | 0.089 |
| | 3 and | 90 | 13.5 | 1.72-106 | 0.014 |
| | above | | | | |

TABLE 9
Survival Analysis (Metastasis) for Genes Showing Co-Deletion With
ZNRF3 In At LeastOne CPC-GENE Case and RNA-CNA Concordance

| symbol | ID | HR | p.value |
|---|---|---|---|
| LIMK2 | NM_016733; NM_005569 | 5.6 | 1.30E−05 |
| KREMEN1 | NM_001039570; NM_032045 | 5.9 | 1.30E−06 |
| ASCC2 | NM_001242906 | 5.3 | 1.20E−05 |
| HSCB | NM_172002 | 5.9 | 5.20E−07 |
| RNF185 | NM_152267 | 5.6 | 1.30E−05 |
| EWSR1 | NM_005243 | 5.6 | 2.80E−06 |
| ZNRF3 | NM_001206998 | 5.9 | 5.20E−07 |
| XBP1 | NM_005080 | 5.9 | 5.20E−07 |
| CCDC117 | NM_173510 | 5.9 | 5.20E−07 |
| RHBDD3 | NM_012265 | 5.6 | 2.80E−06 |
| APOBEC3D | NM_152426 | | |
| UQCR10 | NM_013387 | | |
| MTMR3 | NM_153051 | | |
| HORMAD2 | NM_152510 | | |
| CHEK2 | NM_001257387 | | |
| TTC28 | NM_001145418 | | |
| ZMAT5 | NM_019103 | | |
| EMID1 | NM_001267895 | | |
| ZNRF3.AS1 | NR_046851 | | |
| NIPSNAP1 | NM_003634 | | |
| APOBEC3C | NM_014508 | | |
| APOBEC3F | NM_145298 | | |
| APOBEC3G | NM_021822 | | |
| MIR3928 | NR_037496 | | |
| APOBEC3H | NM_001166002 | | |
| APOBEC3A | NM_001270406 | | |
| C22orf31 | NM_015370 | | |
| CABP7 | NM_182527 | | |
| SYNGR1 | NM_004711 | | |

TABLE 10A
Survival Analysis with RNA Abundance of Genes
Co-Deleted With ZNRF3 (CPCG-BCR)

| | | Upper | Lower | | |
|---|---|---|---|---|---|
| Gene | HR | 95% CI | 95% CI | P-Value | Q-Value |
| LIMK2 | 0.685 | 2.49 | 0.076 | 0.566 | 0.566 |
| KREMEN1 | 0.252 | 0.833 | 0.076 | 0.0238 | 0.0428 |
| ASCC2 | 0.276 | 1.09 | 0.0698 | 0.0665 | 0.0998 |
| HSCB | 0.233 | 0.739 | 0.0735 | 0.0133 | 0.0428 |
| RNF185 | 0.377 | 1.15 | 0.1239 | 0.0855 | 0.11 |
| EWSR1 | 0.371 | 1.33 | 0.103 | 0.128 | 0.144 |
| ZNRF3 | 0.198 | 0.731 | 0.0538 | 0.0151 | 0.0428 |
| XBP1 | 0.376 | 0.719 | 0.197 | 3.11E−03 | 2.80E−02 |
| CCDC117 | 0.296 | 0.823 | 0.107 | 0.0196 | 0.0428 |

TABLE 10B
Survival Analysis with RNA Abundance of Genes Co-Deleted
With ZNRF3 (TCGA-PFS)

| | | Upper | Lower | | |
|---|---|---|---|---|---|
| Gene | HR | 95% CI | 95% CI | P-Value | Q-Value |
| LIMK2 | 0.888 | 1.09 | 0.727 | 0.248 | 0.279 |
| KREMEN1 | 0.734 | 0.951 | 0.567 | 0.0192 | 0.0347 |
| ASCC2 | 0.863 | 1.06 | 0.704 | 0.157 | 0.202 |
| HSCB | 1.06 | 1.3 | 0.866 | 0.569 | 0.569 |
| RNF185 | 0.517 | 0.651 | 0.41 | 2.20E−08 | 1.98E−07 |
| EWSR1 | 1.4 | 1.72 | 1.15 | 9.00E−04 | 0.00405 |
| ZNRF3 | 0.736 | 0.952 | 0.569 | 0.0194 | 0.0347 |
| XBP1 | 0.747 | 0.961 | 0.581 | 2.31E−02 | 3.47E−02 |
| CCDC117 | 0.754 | 0.958 | 0.594 | 0.0207 | 0.0347 |

TABLE 10C
Survival Analysis with RNA Abundance of Genes Co-Deleted
With ZNRF3 (EPOC-BCR)

| | | Upper | Lower | | |
|---|---|---|---|---|---|
| Gene | HR | 95% CI | 95% CI | P-Value | Q-Value |
| LIMK2 | 0.863 | 1 | 0.746 | 0.0492 | 0.11 |
| KREMEN1 | 0.636 | 1.01 | 0.567 | 0.0613 | 0.11 |
| ASCC2 | 0.979 | 1.08 | 0.887 | 0.675 | 0.675 |
| HSCB | 0.906 | 1.02 | 0.805 | 0.105 | 0.158 |
| RNF185 | 0.517 | 0.651 | 0.41 | 1.13E−05 | 5.09E−05 |
| EWSR1 | 0.921 | 1.04 | 0.813 | 1.98E−01 | 0.255 |
| ZNRF3 | 0.436 | 0.61 | 0.311 | 1.27E−06 | 1.14E−05 |
| XBP1 | 0.9998 | 1 | 0.994 | 2.86E−01 | 3.22E−01 |
| CCDC117 | 0.858 | 0.992 | 0.741 | 0.0384 | 0.11 |

TABLE 10D
Survival Analysis with RNA Abundance of Genes Co-Deleted
With ZNRF3-LTRI-BCR)

| | | Upper | Lower | | |
|---|---|---|---|---|---|
| Gene | HR | 95% CI | 95% CI | P-Value | Q-Value |
| LIMK2 | 0.527 | 1.11 | 0.252 | 0.09 | 0.203 |
| KREMEN1 | 0.204 | 0.689 | 0.06 | 0.011 | 0.05 |
| ASCC2 | 0.838 | 2.04 | 0.344 | 0.698 | 0.788 |
| HSCB | 0.875 | 2.32 | 0.33 | 0.788 | 0.788 |
| RNF185 | 0.488 | 2.82 | 0.0844 | 1.16E−01 | 2.09E−01 |
| EWSR1 | 0.762 | 1.62 | 0.358 | 4.80E−01 | 0.72 |
| ZNRF3 | 0.176 | 0.525 | 0.059 | 0.00184 | 0.0166 |
| XBP1 | 0.557 | 0.993 | 0.312 | 4.70E−02 | 1.41E−01 |
| CCDC117 | 0.871 | 1.81 | 0.42 | 0.71 | 0.788 |

TABLE 11A
Multivariable Cox Proportional Hazards Models of
ZNRF3, APC, and CTNNB1 in TCGA
TCGA

| Gene | Level | N | HR (95% CI) | P-Value |
|---|---|---|---|---|
| ZNRF3 | Neutral | 431 | — | — |
| | Loss | 58 | 2.35 (1.41-3.92) | 1.08E−03 |
| APC | Neutral | 422 | — | — |
| | Loss | 67 | 1.97 (1.19-3.25) | 8.15E−03 |
| CTNNB1 | Neutral | 449 | — | — |
| | Loss | 40 | 1.18 (0.597-2.31) | 0.641 |

TABLE 11B
Multivariable Cox Proportional Hazards Models of
ZNRF3, APC, and CTNNB1 in CPC-GENE
CPC-GENE

| Gene | Level | N | HR (95% CI) | | P-Value |
|---|---|---|---|---|---|
| ZNRF3 | Neutral | 349 | — | | — |
| | Loss | 27 | 5.47 | (2.53-11.8) | 1.45E−05 |
| APC | Neutral | 345 | — | | — |
| | Loss | 34 | 1.32 | (0.464-3.73) | 0.606 |
| CTNNB1 | Neutral | 369 | — | | — |
| | Loss | 11 | 0 | (0-Inf) | 1 |

TABLE 12
WNT Pathway Genes Differentially Abundant in CPC-GENE and
TCGA Cases with ZNRF3 Loss

| gene.id | gene.symbol | log2.fold.control | p.value | log10.pval |
|---|---|---|---|---|
| 83999 | KREMEN1 | −0.313872701 | 0.00016057 | 3.794337324 |
| 2487 | FRZB | 0.793632518 | 0.00117591 | 2.929626134 |
| 84133 | ZNRF3 | −0.223141772 | 0.00454452 | 2.342511966 |
| 1277 | COL1A1 | 0.478023086 | 0.00853995 | 2.068544885 |
| 26585 | GREM1 | 0.425329395 | 0.00920713 | 2.035875659 |
| 51176 | LEF1 | 0.545516237 | 0.01194974 | 1.922641505 |
| 10213 | PSMD14 | 0.339816566 | 0.01607545 | 1.793836762 |
| 23240 | TMEM131L | 0.324279969 | 0.03801066 | 1.420094609 |
| 55897 | MESP1 | −0.203779227 | 0.03859672 | 1.413449591 |
| 5682 | PSMA1 | 0.271003506 | 0.04660944 | 1.331526074 |
| 6424 | SFRP4 | 0.547582972 | 0.04812217 | 1.317654828 |
| 56998 | CTNNBIP1 | −0.116363238 | 0.04935941 | 1.306630025 |

All of the methods disclosed and claimed herein can be made and executed without undue experimentation in light of the present disclosure. While the compositions and methods of this invention have been described in terms of certain aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Zhou, C. K. et al. Prostate cancer incidence in 43 populations worldwide: An analysis of time trends overall and by age group. Int. J. Cancer 138, 1388-1400 (2016).
2. Beesley, L. J. et al. Individual and Population Comparisons of Surgery and Radiotherapy Outcomes in Prostate Cancer Using Bayesian Multistate Models. JAMA Netw. open 2, e187765 (2019).
3. Albertsen, P. C., Hanley, J. A., Penson, D. F., Barrows, G. & Fine, J. 13-Year Outcomes Following Treatment for Clinically Localized Prostate Cancer in a Population Based Cohort. J. Urol. 177, 932-936 (2007).
4. D'Amico, A. V. et al. Biochemical outcome after radical prostatectomy, external beam radiation therapy, or interstitial radiation therapy for clinically localized prostate cancer. JAMA 280, 969-974 (1998).
5. Jackson, W. C. et al. Intermediate Endpoints After Postprostatectomy Radiotherapy: 5-Year Distant Metastasis to Predict Overall Survival. Eur. Urol. 0, 413-419 (2018).
6. Buyyounouski, M. K., Pickles, T., Kestin, L. L., Allison, R. & Williams, S. G. Validating the interval to biochemical failure for the identification of potentially lethal prostate cancer. J Clin Oncol 30, 1857-1863 (2012).
7. Fraser, M. et al. Genomic hallmarks of localized, non-indolent prostate cancer. Nature 541, (2017).
8. Wedge, D. C. et al. Sequencing of prostate cancers identifies new cancer genes, routes of progression and drug targets. Nat. Genet. 50, 682-692 (2018).
9. Cooper, C. S. et al. Analysis of the genetic phylogeny of multifocal prostate cancer identifies multiple independent clonal expansions in neoplastic and morphologically normal prostate tissue. Nat Genet 47, 367-372 (2015).
10. Boutros, P. C. et al. Spatial genomic heterogeneity within localized, multifocal prostate cancer. Nat Genet 47, 736-745 (2015).
11. Baca, S. C. et al. Punctuated evolution of prostate cancer genomes. Cell 153, 666-677 (2013).
12. Berger, M. F. et al. The genomic complexity of primary human prostate cancer. Nature 470, 214-220 (2011).
13. Weischenfeldt, J. et al. Integrative Genomic Analyses Reveal an Androgen-Driven Somatic Alteration Landscape in Early-Onset Prostate Cancer. Cancer Cell 23, 159-170 (2013).
14. Gerhauser, C. et al. Molecular Evolution of Early-Onset Prostate Cancer Identifies Molecular Risk Markers and Clinical Trajectories. Cancer Cell 34, 996-1011.e8 (2018).
15. Espiritu, S. M. G. et al. The Evolutionary Landscape of Localized Prostate Cancers Drives Clinical Aggression. Cell 173, (2018).
16. Armenia, J. et al. The long tail of oncogenic drivers in prostate cancer. Nat. Genet. 50, 645-651 (2018).
17. van Dessel, L. F. et al. The genomic landscape of metastatic castration-resistant prostate cancers reveals multiple distinct genotypes with potential clinical impact. Nat. Commun. 10, 1-13 (2019).
18. Quigley, D. A. et al. Genomic Hallmarks and Structural Variation in Metastatic Prostate Cancer. Cell 174, 758-769.e9 (2018).
19. Bhandari, V. et al. Molecular landmarks of tumor hypoxia across cancer types. Nat. Genet. 51, (2019).
20 Espiritu, S. M. G. et al. The Evolutionary Landscape of Localized Prostate Cancers Drives Clinical Aggression. Cell 173, 1003-1013.e15 (2018).
21. Houlahan, K. E. K. E. et al. Genome-wide germline correlates of the epigenetic landscape of prostate cancer. Nat. Med. 25, 1615-1626 (2019).
22. Sinha, A. et al. The Proteogenomic Landscape of Curable Prostate Cancer. Cancer Cell 35, 414-427.e6 (2019).
23. Cancer Genome Atlas Research, N. et al. The Molecular Taxonomy of Primary Prostate Cancer. Cell 163, 1011-1025 (2015).
24. Cerami, E. et al. The cBio Cancer Genomics Portal: An open platform for exploring multidimensional cancer genomics data. Cancer Discov. (2012) doi: 10.1158/2159-8290.CD-12-0095.
25. Gao, J. et al. Integrative analysis of complex cancer genomics and clinical profiles using the cBioPortal. Sci. Signal. (2013) doi: 10.1126/scisignal.2004088.
26 Barbieri, C. E. et al. Exome sequencing identifies recurrent SPOP, FOXA1 and MED12 mutations in prostate cancer. Nat Genet 44, 685-689 (2012).
27. Robinson, D. et al. Integrative clinical genomics of advanced prostate cancer. Cell 161, 1215-1228 (2015).
28 Abida, W. et al. Genomic correlates of clinical outcome in advanced prostate cancer. Proc Natl Acad Sci USA 116, 11428-11436 (2019).
29. Taylor, B. S. et al. Integrative genomic profiling of human prostate cancer. Cancer Cell 18, 11-22 (2010).
30. Lalonde, E. et al. Tumour genomic and microenvironmental heterogeneity for integrated prediction of 5-year biochemical recurrence of prostate cancer: A retrospective cohort study. Lancet Oncol. 15, 1521-1532 (2014).
31. Lalonde, E. et al. Translating a Prognostic DNA Genomic Classifier into the Clinic: Retrospective Validation in 563 Localized Prostate Tumors. Eur. Urol. 72, 22-31 (2017).
32 Hieronymus, H. et al. Tumor copy number alteration burden is a pan-cancer prognostic factor associated with recurrence and death. Elife 7, (2018).
33. Chen, X. et al. Manta: rapid detection of structural variants and indels for germline and cancer sequencing applications. Bioinformatics 32, 1220-1222 (2016).
34 Harrow, J. et al. GENCODE: The reference human genome annotation for the ENCODE project. Genome Res. 22, 1760-1774 (2012).
35. Robinson, M. D., McCarthy, D. J. & Smyth, G. K. edgeR: a Bioconductor package for differential expression analysis of digital gene expression data. Bioinformatics 26, 139-140 (2010).
36. Roach 3rd, M. et al. Defining biochemical failure following radiotherapy with or without hormonal therapy in men with clinically localized prostate cancer: recommendations of the RTOG-ASTRO Phoenix Consensus Conference. Int J Radiat Oncol Biol Phys 65, 965-974 (2006).
37 Liu, J. et al. An Integrated TCGA Pan-Cancer Clinical Data Resource to Drive High-Quality Survival Outcome Analytics. Cell 173, 400-416.e11 (2018).
38. Canter, D. J. et al. Analysis of the prognostic utility of the cell cycle progression (CCP) score generated from needle biopsy in men treated with definitive therapy. Prostate Cancer Prostatic Dis. (2019) doi: 10.1038/s41391-019-0159-9.
39. Cuzick, J. et al. Prognostic value of an RNA expression signature derived from cell cycle proliferation genes in patients with prostate cancer: A retrospective study. Lancet Oncol. 12, 245-55 (2011).
40. P′ng, C. et al. BPG: Seamless, automated and interactive visualization of scientific data. BMC Bioinformatics 20, 42 (2019).
41. Lam, F. et al. VennDiagramWeb: a web application for the generation of highly customizable Venn and Euler diagrams. BMC Bioinformatics 17, 401 (2016).
42. Grasso, C. S. et al. The mutational landscape of lethal castration-resistant prostate cancer. Nature 487, 239-243 (2012).
43. Mateo, J. et al. DNA-Repair Defects and Olaparib in Metastatic Prostate Cancer. N Engl J Med 373, 1697-1708 (2015).
44 Pritchard, C. C. et al. Inherited DNA-Repair Gene Mutations in Men with Metastatic Prostate Cancer. N Engl J Med 375, 443-453 (2016).
45. Boysen, G. et al. SPOP mutated/CHD1 deleted lethal prostate cancer and abiraterone sensitivity. Clin Cancer Res (2018) doi: 10.1158/1078-0432.CCR-18-0937.
46. Ishkanian, A. S. et al. High-resolution array CGH identifies novel regions of genomic alteration in intermediate-risk prostate cancer. Prostate 69, 1091-1100 (2009).
47. Goodwin, J. F. et al. DNA-PKcs-Mediated Transcriptional Regulation Drives Prostate Cancer Progression and Metastasis. Cancer Cell 28, 97-113 (2015).
48. Chung, J. H. et al. Prospective Comprehensive Genomic Profiling of Primary and Metastatic Prostate Tumors. JCO Precis. Oncol. 3, 1-23 (2019).
49. Ding, Z. et al. SMAD4-dependent barrier constrains prostate cancer growth and metastatic progression. Nature 470, 269-273 (2011).
50. Gerhauser, C. et al. Molecular Evolution of Early-Onset Prostate Cancer Identifies Molecular Risk Markers and Clinical Trajectories Cancer Cell Article Molecular Evolution of Early-Onset Prostate Cancer Identifies Molecular Risk Markers and Clinical Trajectories. Cancer Cell 34, 996-1011.e8 (2018).
51. Chua, M. L. K. et al. A Prostate Cancer “Nimbosus”: Genomic Instability and SChLAP1 Dysregulation Underpin Aggression of Intraductal and Cribriform Subpathologies[Figure presented]. Eur. Urol. 72, 665-674 (2017).
52. Van Der Kwast, T. et al. Biopsy diagnosis of intraductal carcinoma is prognostic in intermediate and high risk prostate cancer patients treated by radiotherapy. Eur. J. Cancer 48, 1318-1325 (2012).
53. Siadat, F. et al. Not all gleason pattern 4 prostate cancers are created equal: A study of latent prostatic carcinomas in a cystoprostatectomy and autopsy series. Prostate 75, 1277-1284 (2015).
54. Trudel, D. et al. Prognostic impact of intraductal carcinoma and large cribriform carcinoma architecture after prostatectomy in a contemporary cohort. Eur. J. Cancer 50, 1610-1616 (2014).
55. Kweldam, C. F. et al. Cribriform growth is highly predictive for postoperative metastasis and disease-specific death in Gleason score 7 prostate cancer. Mod Pathol 28, 457-464 (2015).
56. Kweldam, C. F. et al. Presence of invasive cribriform or intraductal growth at biopsy outperforms percentage grade 4 in predicting outcome of Gleason score 3+4=7 prostate cancer. Mod Pathol (2017) doi: 10.1038/modpathol.2017.29.
57. Milosevic, M. et al. Tumor hypoxia predicts biochemical failure following radiotherapy for clinically localized prostate cancer. Clin. Cancer Res. (2012) doi: 10.1158/1078-0432.CCR-11-2711.
58 Chandran, U. R. et al. Gene expression profiles of prostate cancer reveal involvement of multiple molecular pathways in the metastatic process. BMC Cancer (2007) doi: 10.1186/1471-2407-7-64.
59. Kron, K. J. et al. DNA methylation of HOXD3 as a marker of prostate cancer progression. Lab Invest 90, 1060-1067 (2010).
60. Olkhov-Mitsel, E. et al. Distinct DNA methylation alterations are associated with cribriform architecture and intraductal carcinoma in Gleason pattern 4 prostate tumors. Oncol Lett 14, 390-396 (2017).
61. Hamid, A. A. et al. Compound Genomic Alterations of TP53, PTEN, and RB1 Tumor Suppressors in Localized and Metastatic Prostate Cancer. Eur. Urol. 76, 89-97 (2019).
62. Taylor, R. A. et al. The influence of BRCA2 mutation on localized prostate cancer. Nat. Rev. Urol. (2019) doi: 10.1038/s41585-019-0164-8.
63. Taylor, R. A. et al. Germline BRCA2 mutations drive prostate cancers with distinct evolutionary trajectories. Nat Commun 8, 13671 (2017).
64. Risbridger, G. P. et al. Patient-derived xenografts reveal that intraductal carcinoma of the prostate is a prominent pathology in brca2 mutation carriers with prostate cancer and correlates with poor prognosis. Eur. Urol. 67, 496-503 (2015).
65. Liu, J. et al. Targeting Wnt-driven cancer through the inhibition of Porcupine by LGK974. Proc. Natl. Acad. Sci. U.S.A 110, 20224-20229 (2013).
66. Feng, F. Y. et al. Validation of a 22-Gene Genomic Classifier in Patients With Recurrent Prostate Cancer. JAMA Oncol. (2021) doi: 10.1001/jamaoncol.2020.7671.
67. Bailey, M. H. M. H. et al. Retrospective evaluation of whole exome and genome mutation calls in 746 cancer samples. Nat. Commun. 11, 17 (2020).
68. Liu, L. Y. et al. Quantifying the influence of mutation detection on tumour subclonal reconstruction. Nat. Commun. 11, 1-15 (2020).
69. Campbell, P. J. P. J. et al. Pan-cancer analysis of whole genomes. Nature 578, 82-93 (2020).

What is claimed is:

1. A method for treating a subject for prostate cancer, the method comprising administering an effective amount of a prostate cancer therapy to a subject determined to have ZNRF3 genomic loss in a prostate cancer sample from the subject.

2. The method of claim 1, wherein the prostate cancer therapy comprises chemotherapy, cryoablative therapy, hi-intensity ultrasound, photodynamic therapy, laser ablation, irreversible electroporation, hormone therapy, radiotherapy, surgery, immunotherapy, or a combination thereof.

3. The method of claim 2, wherein the prostate cancer therapy comprises radiation and hormone therapy.

4. The method of any of claims 1-3, wherein the subject was further determined to have prostate cancer with CCND1 genomic gain.

5. The method of any of claims 1-4, wherein the subject was diagnosed with very low risk prostate cancer.

6. The method of any of claims 1-4, wherein the subject was diagnosed with low risk prostate cancer.

7. The method of any of claims 1-4, wherein the subject was diagnosed with intermediate favorable risk prostate cancer.

8. The method of any of claims 1-4, wherein the subject was diagnosed with intermediate unfavorable risk prostate cancer.

9. The method of any of claims 1-4, wherein the subject was diagnosed with high risk prostate cancer.

10. The method of any of claims 1-4, wherein the subject was diagnosed with very high risk prostate cancer.

11. The method of any of claims 1-4, wherein the subject was diagnosed with metastatic prostate cancer.

12. The method of any of claims 1-11, wherein the prostate cancer sample is a tissue sample.

13. The method of any of claims 1-11, wherein the prostate cancer sample is a blood sample.

14. The method of any of claims 1-11, wherein the prostate cancer sample is a plasma sample.

15. The method of any of claims 1-11, wherein the prostate cancer sample is a urine sample.

16. The method of any of claims 1-11, wherein the prostate cancer sample is a semen sample.

17. The method of any of claims 1-11, wherein the prostate cancer sample is a sample of circulating tumor cells.

18. The method of any of claims 1-11, wherein the prostate cancer sample is a cell-free nucleic acid sample.

19. The method of any of claims 1-18, wherein the subject was determined to have ZNRF3 genomic loss by sequencing DNA from the prostate cancer sample.

20. The method of any of claims 1-18, wherein the subject was determined to have ZNRF3 genomic loss by polymerase chain reaction analysis of DNA from the prostate cancer sample.

21. The method of any of claims 1-18, wherein the subject was determined to have ZNRF3 genomic loss by microarray analysis of DNA from the prostate cancer sample.

22. The method of any of claims 1-18, wherein the subject was determined to have ZNRF3 genomic loss by in situ hybridization analysis of DNA from the prostate cancer sample.

23. A method for treating a subject for prostate cancer, the method comprising administering an effective amount of a prostate cancer therapy to a subject determined to have reduced ZNRF3 expression in a prostate cancer sample from the subject.

24. The method of claim 23, wherein the prostate cancer therapy comprises chemotherapy, cryoablative therapy, hi-intensity ultrasound, photodynamic therapy, laser ablation, irreversible electroporation, hormone therapy, radiotherapy, surgery, immunotherapy, or a combination thereof.

25. The method of claim 24, wherein the prostate cancer therapy comprises radiation and hormone therapy.

26. The method of any of claims 23-25, wherein the subject was further determined to have prostate cancer with CCND1 genomic gain.

27. The method of any of claims 23-26, wherein the subject was diagnosed with very low risk prostate cancer.

28. The method of any of claims 23-26, wherein the subject was diagnosed with low risk prostate cancer.

29. The method of any of claims 23-26, wherein the subject was diagnosed with intermediate favorable risk prostate cancer.

30. The method of any of claims 23-26, wherein the subject was diagnosed with intermediate unfavorable risk prostate cancer.

31. The method of any of claims 23-26, wherein the subject was diagnosed with high risk prostate cancer.

32. The method of any of claims 23-26, wherein the subject was diagnosed with very high risk prostate cancer.

33. The method of any of claims 23-26, wherein the subject was diagnosed with metastatic prostate cancer.

34. The method of any of claims 23-33, wherein the prostate cancer sample is a tissue sample.

35. The method of any of claims 23-33, wherein the prostate cancer sample is a blood sample.

36. The method of any of claims 23-33, wherein the prostate cancer sample is a plasma sample.

37. The method of any of claims 23-33, wherein the prostate cancer sample is a urine sample.

38. The method of any of claims 23-33, wherein the prostate cancer sample is a semen sample.

39. The method of any of claims 23-33, wherein the prostate cancer sample is a sample of circulating tumor cells.

40. The method of any of claims 23-33, wherein the prostate cancer sample is a cell-free nucleic acid sample.

41. The method of any of claims 23-40, wherein the reduced ZNRF3 expression is reduced ZNRF3 RNA.

42. The method of claim 41, wherein the subject was determined to have reduced ZNRF3 expression by sequencing RNA from the prostate cancer sample.

43. The method of any of claims 23-40, wherein the reduced ZNRF3 expression is reduced ZNRF3 protein.

44. The method of claim 43, wherein the subject was determined to have reduced ZNRF3 expression by measuring ZNRF3 protein levels from the prostate cancer sample.

45. A method for treating a subject for prostate cancer, the method comprising administering an effective amount of a prostate cancer therapy to a subject determined to have increased ZNRF3 methylation in a prostate cancer sample from the subject.

46. The method of claim 45, wherein the prostate cancer therapy comprises chemotherapy, cryoablative therapy, hi-intensity ultrasound, photodynamic therapy, laser ablation, irreversible electroporation, hormone therapy, radiotherapy, surgery, immunotherapy, or a combination thereof.

47. The method of claim 46, wherein the prostate cancer therapy comprises radiation and hormone therapy.

48. The method of any of claims 45-47, wherein the subject was further determined to have prostate cancer with CCND1 genomic gain.

49. The method of any of claims 45-48, wherein the subject was diagnosed with very low risk prostate cancer.

50. The method of any of claims 46-48, wherein the subject was diagnosed with low risk prostate cancer.

51. The method of any of claims 46-48, wherein the subject was diagnosed with intermediate favorable risk prostate cancer.

52. The method of any of claims 46-48, wherein the subject was diagnosed with intermediate unfavorable risk prostate cancer.

53. The method of any of claims 46-48, wherein the subject was diagnosed with high risk prostate cancer.

54. The method of any of claims 46-48, wherein the subject was diagnosed with very high risk prostate cancer.

55. The method of any of claims 46-48, wherein the subject was diagnosed with metastatic prostate cancer.

56. The method of any of claims 46-55, wherein the prostate cancer sample is a tissue sample.

57. The method of any of claims 46-55, wherein the prostate cancer sample is a blood sample.

58. The method of any of claims 46-55, wherein the prostate cancer sample is a plasma sample.

59. The method of any of claims 46-55, wherein the prostate cancer sample is a urine sample.

60. The method of any of claims 46-55, wherein the prostate cancer sample is a semen sample.

61. The method of any of claims 46-55, wherein the prostate cancer sample is a sample of circulating tumor cells.

62. The method of any of claims 46-55, wherein the prostate cancer sample is a cell-free nucleic acid sample.

63. The method of any of claims 46-62, wherein the subject was determined to have increased ZNRF3 methylation by sequencing DNA from the prostate cancer sample.

64. The method of claim 63, wherein the sequencing comprised bisulfite sequencing.

65. The method of any of claims 46-62, wherein the subject was determined to have increased ZNRF3 methylation by polymerase chain reaction analysis of DNA from the prostate cancer sample.

66. The method of any of claims 46-62, wherein the subject was determined to have increased ZNRF3 methylation by microarray analysis of DNA from the prostate cancer sample.

67. The method of any of claims 46-62, wherein the subject was determined to have increased ZNRF3 methylation by in situ hybridization analysis of DNA from the prostate cancer sample.

68. A method for treating a subject for prostate cancer, the method comprising:
(a) detecting ZNRF3 genomic loss in a prostate cancer sample from the subject; and
(b) administering an effective amount of a prostate cancer therapy to the subject.

69. The method of claim 68, wherein the prostate cancer therapy comprises chemotherapy, cryoablative therapy, hi-intensity ultrasound, photodynamic therapy, laser ablation, irreversible electroporation, hormone therapy, radiotherapy, surgery, immunotherapy, or a combination thereof.

70. The method of claim 69, wherein the prostate cancer therapy comprises radiation and hormone therapy.

71. The method of any of claims 68-70, further comprising detecting CCND1 genomic gain in the prostate cancer sample.

72. The method of any of claims 68-71, wherein the subject was diagnosed with very low risk prostate cancer.

73. The method of any of claims 68-71, wherein the subject was diagnosed with low risk prostate cancer.

74. The method of any of claims 68-71, wherein the subject was diagnosed with intermediate favorable risk prostate cancer.

75. The method of any of claims 68-71, wherein the subject was diagnosed with intermediate unfavorable risk prostate cancer.

76. The method of any of claims 68-71, wherein the subject was diagnosed with high risk prostate cancer.

77. The method of any of claims 68-71, wherein the subject was diagnosed with very high risk prostate cancer.

78. The method of any of claims 68-71, wherein the subject was diagnosed with metastatic prostate cancer.

79. The method of any of claims 68-78, wherein the prostate cancer sample is a tissue sample.

80. The method of any of claims 68-78, wherein the prostate cancer sample is a blood sample.

81. The method of any of claims 68-78, wherein the prostate cancer sample is a plasma sample.

82. The method of any of claims 68-78, wherein the prostate cancer sample is a urine sample.

83. The method of any of claims 68-78, wherein the prostate cancer sample is a semen sample.

84. The method of any of claims 68-78, wherein the prostate cancer sample is a sample of circulating tumor cells.

85. The method of any of claims 68-78, wherein the prostate cancer sample is a cell-free nucleic acid sample.

86. The method of any of claims 68-85, wherein (a) comprises sequencing DNA from the prostate cancer sample.

87. The method of any of claims 68-85, wherein (a) comprises polymerase chain reaction.

88. The method of any of claims 68-85, where (a) comprises microarray analysis.

89. The method of any of claims 68-85, where (a) comprises in situ hybridization.

90. A method for treating a subject for prostate cancer, the method comprising:
(a) detecting reduced ZNRF3 expression in a prostate cancer sample from the subject; and
(b) administering an effective amount of a prostate cancer therapy to the subject.

91. The method of claim 90, wherein the prostate cancer therapy comprises chemotherapy, cryoablative therapy, hi-intensity ultrasound, photodynamic therapy, laser ablation, irreversible electroporation, hormone therapy, radiotherapy, surgery, immunotherapy, or a combination thereof.

92. The method of claim 91, wherein the prostate cancer therapy comprises radiation and hormone therapy.

93. The method of any of claims 90-92, further comprising detecting CCND1 genomic gain in the prostate cancer sample.

94. The method of any of claims 90-93, wherein the subject was diagnosed with very low risk prostate cancer.

95. The method of any of claims 90-93, wherein the subject was diagnosed with low risk prostate cancer.

96. The method of any of claims 90-93, wherein the subject was diagnosed with intermediate favorable risk prostate cancer.

97. The method of any of claims 90-93, wherein the subject was diagnosed with intermediate unfavorable risk prostate cancer.

98. The method of any of claims 90-93, wherein the subject was diagnosed with high risk prostate cancer.

99. The method of any of claims 90-93, wherein the subject was diagnosed with very high risk prostate cancer.

100. The method of any of claims 90-93, wherein the subject was diagnosed with metastatic prostate cancer.

101. The method of any of claims 90-100, wherein the prostate cancer sample is a tissue sample.

102. The method of any of claims 90-100, wherein the prostate cancer sample is a blood sample.

103. The method of any of claims 90-100, wherein the prostate cancer sample is a plasma sample.

104. The method of any of claims 90-100, wherein the prostate cancer sample is a urine sample.

105. The method of any of claims 90-100, wherein the prostate cancer sample is a semen sample.

106. The method of any of claims 90-100, wherein the prostate cancer sample is a sample of circulating tumor cells.

107. The method of any of claims 90-100, wherein the prostate cancer sample is a cell-free nucleic acid sample.

108. The method of any of claims 90-107, wherein the reduced ZNRF3 expression is reduced ZNRF3 RNA.

109. The method of claim 108, wherein (a) comprises sequencing RNA from the prostate cancer sample.

110. The method of any of claims 90-107, wherein the reduced ZNRF3 expression is reduced ZNRF3 protein.

111. The method of claim 110, wherein (a) comprises measuring ZNRF3 protein levels from the prostate cancer sample.

112. A method for treating a subject for prostate cancer, the method comprising:
(a) detecting increased ZNRF3 methylation in a prostate cancer sample from the subject; and
(b) administering an effective amount of a prostate cancer therapy to the subject.

113. The method of claim 112, wherein the prostate cancer therapy comprises chemotherapy, cryoablative therapy, hi-intensity ultrasound, photodynamic therapy, laser ablation, irreversible electroporation, hormone therapy, radiotherapy, surgery, immunotherapy, or a combination thereof.

114. The method of claim 113, wherein the prostate cancer therapy comprises radiation and hormone therapy.

115. The method of any of claims 112-114, further comprising detecting CCND1 genomic gain in the prostate cancer sample.

116. The method of any of claims 112-115, wherein the subject was diagnosed with very low risk prostate cancer.

117. The method of any of claims 112-115, wherein the subject was diagnosed with low risk prostate cancer.

118. The method of any of claims 112-115, wherein the subject was diagnosed with intermediate favorable risk prostate cancer.

119. The method of any of claims 112-115, wherein the subject was diagnosed with intermediate unfavorable risk prostate cancer.

120. The method of any of claims 112-115, wherein the subject was diagnosed with high risk prostate cancer.

121. The method of any of claims 112-115, wherein the subject was diagnosed with very high risk prostate cancer.

122. The method of any of claims 112-115, wherein the subject was diagnosed with metastatic prostate cancer.

123. The method of any of claims 112-122, wherein the prostate cancer sample is a tissue sample.

124. The method of any of claims 112-122, wherein the prostate cancer sample is a blood sample.

125. The method of any of claims 112-122, wherein the prostate cancer sample is a plasma sample.

126. The method of any of claims 112-122, wherein the prostate cancer sample is a urine sample.

127. The method of any of claims 112-122, wherein the prostate cancer sample is a semen sample.

128. The method of any of claims 112-122, wherein the prostate cancer sample is a sample of circulating tumor cells.

129. The method of any of claims 112-122, wherein the prostate cancer sample is a cell-free nucleic acid sample.

130. The method any of claims 112-129, wherein (a) comprises sequencing ZNRF3 DNA from the prostate cancer sample.

131. The method of claim 130, wherein the sequencing comprises bisulfite sequencing.

132. The method any of claims 112-129, wherein (a) comprises polymerase chain reaction.

133. The method any of claims 112-129, where (a) comprises microarray analysis.

134. The method any of claims 112-129, where (a) comprises in situ hybridization.

135. A method for prostate cancer prognosis, the method comprising (a) detecting ZNRF3 genomic loss in a prostate cancer sample from a subject; and (b) identifying the subject as being at high risk for metastatic prostate cancer.

136. The method of claim 135, further comprising administering to the subject an effective amount of a prostate cancer therapy.

137. The method of claim 136, wherein the prostate cancer therapy comprises chemotherapy, cryoablative therapy, hi-intensity ultrasound, photodynamic therapy, laser ablation, irreversible electroporation, hormone therapy, radiotherapy, surgery, immunotherapy, or a combination thereof.

138. The method of claim 137, wherein the prostate cancer therapy comprises radiation and hormone therapy.

139. The method of any of claims 135-138, further comprising, prior to (b), detecting CCND1 genomic gain in the prostate cancer sample.

140. A method for prostate cancer prognosis, the method comprising (a) detecting reduced ZNRF3 expression in a prostate cancer sample from a subject; and (b) identifying the subject as being at high risk for metastatic prostate cancer.

141. The method of claim 140, further comprising administering to the subject an effective amount of a prostate cancer therapy.

142. The method of claim 141, wherein the prostate cancer therapy comprises chemotherapy, cryoablative therapy, hi-intensity ultrasound, photodynamic therapy, laser ablation, irreversible electroporation, hormone therapy, radiotherapy, surgery, immunotherapy, or a combination thereof.

143. The method of claim 142, wherein the prostate cancer therapy comprises radiation and hormone therapy.

144. The method of any of claims 140-143, further comprising, prior to (b), detecting CCND1 genomic gain in the prostate cancer sample.

145. A method for prostate cancer prognosis, the method comprising (a) detecting increased ZNRF3 methylation in a prostate cancer sample from a subject; and (b) identifying the subject as being at high risk for metastatic prostate cancer.

146. The method of claim 145, further comprising administering to the subject an effective amount of a prostate cancer therapy.

147. The method of claim 146, wherein the prostate cancer therapy comprises chemotherapy, cryoablative therapy, hi-intensity ultrasound, photodynamic therapy, laser ablation, irreversible electroporation, hormone therapy, radiotherapy, surgery, immunotherapy, or a combination thereof.

148. The method of claim 147, wherein the prostate cancer therapy comprises radiation and hormone therapy.

149. The method of any of claims 145-148, further comprising, prior to (b), detecting CCND1 genomic gain in the prostate cancer sample.

150. A method for diagnosing a subject with high risk or very high risk prostate cancer, the method comprising detecting ZNRF3 genomic loss in a prostate cancer sample from the subject.

151. A method for diagnosing a subject with high risk or very high risk prostate cancer, the method comprising detecting reduced ZNRF3 expression in a prostate cancer sample from the subject.

152. A method for diagnosing a subject with high risk or very high risk prostate cancer, the method comprising detecting increased ZNRF3 methylation in a prostate cancer sample from the subject.

153. A method for treating a subject for prostate cancer, the method comprising administering an effective amount of a prostate cancer therapy to a subject (a) diagnosed with very low risk, low risk, or intermediate favorable risk prostate cancer and (b) determined to have prostate cancer with ZNRF3 genomic loss.

154. A method for treating a subject for prostate cancer, the method comprising administering an effective amount of a prostate cancer therapy to a subject (a) diagnosed with very low risk, low risk, or intermediate favorable risk prostate cancer and (b) determined to have prostate cancer with reduced ZNRF3 expression.

155. The method of claim 154, wherein the reduced ZNRF3 expression is reduced ZNRF3 RNA.

156. The method of claim 154, wherein the reduced ZNRF3 expression is reduced ZNRF3 protein.

157. A method for treating a subject for prostate cancer, the method comprising administering an effective amount of a prostate cancer therapy to a subject (a) diagnosed with very low risk, low risk, or intermediate favorable risk prostate cancer and (b) determined to have prostate cancer with increased ZNRF3 methylation.

158. A method for treating a subject for prostate cancer, the method comprising administering an effective amount of radiotherapy and hormone therapy to a subject (a) diagnosed with intermediate unfavorable risk prostate cancer and (b) determined to have prostate cancer with ZNRF3 genomic loss.

159. A method for treating a subject for prostate cancer, the method comprising administering an effective amount of radiotherapy and hormone therapy to a subject (a) diagnosed with intermediate unfavorable risk prostate cancer and (b) determined to have prostate cancer with reduced ZNRF3 expression.

160. A method for treating a subject for prostate cancer, the method comprising administering an effective amount of radiotherapy and hormone therapy to a subject (a) diagnosed with intermediate unfavorable risk prostate cancer and (b) determined to have prostate cancer with increased ZNRF3 methylation.

161. A method for treating a subject for prostate cancer, the method comprising:
(a) diagnosing a subject with very low risk, low risk, or intermediate favorable risk prostate cancer;
(b) detecting ZNRF3 genomic loss in a prostate cancer sample from the subject; and
(c) administering an effective amount of a prostate cancer therapy to the subject.

162. A method for treating a subject for prostate cancer, the method comprising:
(a) diagnosing a subject with very low risk, low risk, or intermediate favorable risk prostate cancer;
(b) detecting reduced ZNRF3 expression in a prostate cancer sample from the subject; and
(c) administering an effective amount of a prostate cancer therapy to the subject.

163. A method for treating a subject for prostate cancer, the method comprising:
(a) diagnosing a subject with very low risk, low risk, or intermediate favorable risk prostate cancer;
(b) detecting increased ZNRF3 methylation in a prostate cancer sample from the subject; and
(c) administering an effective amount of a prostate cancer therapy to the subject.

164. A method for treating a subject for prostate cancer, the method comprising:
(a) diagnosing a subject with intermediate unfavorable risk prostate cancer;
(b) detecting reduced expression of ZNRF3 in a prostate cancer sample from the subject; and
(c) administering an effective amount of radiotherapy and hormone therapy to the subject.

165. A method for treating a subject for prostate cancer, the method comprising:
(a) diagnosing a subject with intermediate unfavorable risk prostate cancer;
(b) detecting reduced expression of ZNRF3 in a prostate cancer sample from the subject; and
(c) administering an effective amount of radiotherapy and hormone therapy to the subject.

166. A method for treating a subject for prostate cancer, the method comprising:
(a) diagnosing a subject with intermediate unfavorable risk prostate cancer;
(b) detecting reduced expression of ZNRF3 in a prostate cancer sample from the subject; and
(c) administering an effective amount of radiotherapy and hormone therapy to the subject.

167. A method for treating a subject diagnosed with intermediate favorable risk prostate cancer, the method comprising:
(a) determining whether a prostate cancer sample from the subject has ZNRF3 genomic loss; and
(b) if the prostate cancer sample is determined to have ZNRF3 genomic loss, administering an effective amount of a prostate cancer therapy to the subject.

168. A method for treating a subject diagnosed with intermediate favorable risk prostate cancer, the method comprising:
(a) determining whether a prostate cancer sample from the subject has reduced ZNRF3 expression; and
(b) if the prostate cancer sample is determined to have reduced ZNRF3 expression, administering an effective amount of a prostate cancer therapy to the subject.

169. A method for treating a subject diagnosed with intermediate unfavorable risk prostate cancer, the method comprising:
(a) determining whether a prostate cancer sample from the subject has reduced ZNRF3 expression;
(b) if the prostate cancer sample is determined to have reduced ZNRF3 expression, administering an effective amount of radiotherapy and hormone therapy to the subject; and
(c) if the prostate cancer sample is determined not to have reduced ZNRF3 expression, administering an effective amount of a prostate cancer therapy that does not comprise hormone therapy.

170. A method for treating a subject diagnosed with intermediate unfavorable risk prostate cancer, the method comprising:
(a) determining whether a prostate cancer sample from the subject has ZNRF3 genomic loss;
(b) if the prostate cancer sample is determined to have ZNRF3 genomic loss, administering an effective amount of radiotherapy and hormone therapy to the subject; and
(c) if the prostate cancer sample is determined not to have ZNRF3 genomic loss, administering an effective amount of a prostate cancer therapy that does not comprise hormone therapy.

171. A method for treating a subject for prostate cancer, the method comprising administering an effective amount of a prostate cancer therapy to a subject determined to have, in a prostate cancer sample from the subject, one or more of genomic loss of BRCA2, genomic gain of CCND1, genomic loss of CDH1, genomic loss of CDK12, genomic los of CHD1, a structural variation of CHD1, an inversion of chr10:89 Mbp, an inter-chromosomal translocation of chr21:42 Mbp, an inversion of chr3:125 Mbp, genomic gain of ETV1, genomic gain of ETC5, a non-synonymous mutation of MSH2, genomic gain of MYC, genomic loss of NKX3-1, a structural variation of NKX3-1, genomic gain of PRKDC, genomic loss of PTEN, genomic los of RB1, a structural variation of RB1, a non-synonymous mutation of SPOP, genomic loss of TP53, genomic loss of ZBTB 16, genomic los of ZFHX3, and genomic loss of ZNRF3.

172. A method for treating a subject for prostate cancer, the method comprising:
(a) detecting, in a prostate cancer sample from the subject, one or more of genomic loss of BRCA2, genomic gain of CCND1, genomic loss of CDH1, genomic loss of CDK12, genomic los of CHD1, a structural variation of CHD1, an inversion of chr10:89 Mbp, an inter-chromosomal translocation of chr21:42 Mbp, an inversion of chr3:125 Mbp, genomic gain of ETV1, genomic gain of ETC5, a non-synonymous mutation of MSH2, genomic gain of MYC, genomic loss of NKX3-1, a structural variation of NKX3-1, genomic gain of PRKDC, genomic loss of PTEN, genomic los of RB1, a structural variation of RB1, a non-synonymous mutation of SPOP, genomic loss of TP53, genomic loss of ZBTB16, genomic los of ZFHX3, and genomic loss of ZNRF3; and
(b) administering an effective amount of a prostate cancer therapy to the subject.
